SEQUENCE LISTING (10 sequences)

SEQ ID NO: 1 - protein, 15 aa, Artificial Sequence
  peptide fragment
RRRRRRLLLAAAEEE

SEQ ID NO: 2 - protein, 15 aa, Artificial Sequence
  peptide fragment
RRRRRRLLLAAAEEE

SEQ ID NO: 3 - protein, 15 aa, Artificial Sequence
  peptide fragment
KKKKKKLLLAAAEEE

SEQ ID NO: 4 - protein, 13 aa, Artificial Sequence
  peptide fragment
RRRRRLLLAAAEE

SEQ ID NO: 5 - protein, 9 aa, Artificial Sequence
  peptide fragment
RRRLLLEEE

SEQ ID NO: 6 - protein, 13 aa, Artificial Sequence
  peptide fragment
RRRRRLLLEEEEE

SEQ ID NO: 7 - protein, 15 aa, Artificial Sequence
  peptide fragment
RRRRRLLLLLEEEEE

SEQ ID NO: 8 - protein, 21 aa, Artificial Sequence
  peptide fragment
RRRRRRRLLLLLLLEEEEEEE

SEQ ID NO: 9 - protein, 15 aa, Artificial Sequence
  peptide fragment
EEEEELLLLLRRRRR

SEQ ID NO: 10 - protein, 15 aa, Artificial Sequence
  peptide fragment
RRRRRLLLLLDDDDD

(19) United States
(12) Patent Application Publication
(10) Pub. No.: US 2024/0299414 A1
(43) Pub. Date: Sep. 12, 2024

(54) SUBCUTANEOUS OR INTRAMUSCULAR INJECTION FORMULATIONS OF ILOPROST

(71) Applicant: EICOS SCIENCES, INC., Chevy Chase, MD (US)

(72) Inventors: Wade W. BENTON, Emerald Hills, CA (US); Kevin A. CHRISTAL, Burlingame, CA (US)

(21) Appl. No.: 18/260,779

(22) PCT Filed: Aug. 13, 2021
(86) PCT No.: PCT/US2021/045963

§ 371 Date: Jul. 7, 2023

Related U.S. Application Data
(60) Provisional application No. 63/065,118, filed on Aug. 13, 2020.

(51) Int. Cl. A61K 31/5578; A61K 9/00; A61K 47/24; A61P 9/10; A61P 9/14

(52) U.S. Cl. CPC A61K 31/5578; A61K 9/0024; A61K 47/24; A61P 9/10; A61P 9/14

(57) ABSTRACT

The present disclosure generally relates to treatment of systemic sclerosis with symptomatic Raynaud's Phenomenon and/or digital ulcers by subcutaneous or intramuscular injection of iloprost or a pharmaceutically acceptable salt or stereoisomer thereof.

### CROSS-REFERENCE TO RELATED APPLICATIONS

This application claims the benefit of U.S. Provisional Application No. 63/065,118, filed Aug. 13, 2020, the disclosures of which are incorporated by reference herein in its entirety.

### FIELD OF THE INVENTION

The present disclosure generally relates to subcutaneous or intramuscular injection formulation comprising iloprost or a pharmaceutically acceptable salt or stereoisomer thereof. Iloprost injection formulation is useful for treatment of systemic sclerosis with symptomatic Raynaud's Phenomenon and/or digital ulcers.

### STATEMENT REGARDING SEQUENCE LISTING

The sequence listing associated with this application is provided in text format in lieu of a paper copy, and is hereby incorporated by reference into the specification. The name of the text file containing the sequence listing is CIVI_002_01 WO_SeqList_ST25.txt. The text file is ˜2.61 kb, was created on Aug. 13, 2021, and is being submitted electronically via EFS-Web.

### BACKGROUND OF THE INVENTION

The primary limitation of iloprost for the treatment of patients with systemic sclerosis is the need for lengthy intravenous or subcutaneous infusions due to intrinsic pharmacokinetic properties. The parenteral administration is preferred for prostacyclin analogs due their narrow therapeutic window, poor oral route bioavailability, and tolerability. Iloprost injection, for intravenous or subcutaneous use is delivered to systemic circulation at a controlled rate over 6 hours using an infusion pump. The intravenous route of administration is invasive and associated with catheter safety related complications such as infiltration, extravasation and infections. Subcutaneous continuous infusion is associated with high rates of site reactions and site pain. An iloprost parenteral controlled release delivery system offers several advantages including enhanced safety and a considerable reduction in administration time burden. Controlled release systems often use a drug delivery matrix that generally release the drug in a measured and controlled manner within the therapeutic window. Drug delivery systems release kinetic properties are contingent on the physiochemical properties of a drug. The transport of a substance drug within a drug matrix or depot is an important factor for drug release kinetics and is dependent on diffusivity and solubility of the drug in the matrix. Frequently volatile drug substances, like iloprost, undergo degradation in drug delivery systems making formulation development challenging.

Systemic Sclerosis (SSc), also known as scleroderma, is a rare, autoimmune disease with pathogenesis characterized by small vessel vasculopathy primarily affecting small arteries and arterioles, production of autoantibodies, and fibroblast dysfunction leading to increased deposition of extracellular matrix in the skin and internal organs (van de Hoogen et al., 2013; Denton et al., 2017). The clinical manifestations and prognosis of SSc are heterogeneous; most patients have skin thickening and variable involvement of cardiovascular, gastrointestinal (GI), musculoskeletal, and pulmonary systems (Hinchcliff, et al., 2008). Subsets of SSc can be discerned; these include limited cutaneous SSc, diffuse cutaneous SSc, and SSc without skin involvement (sine SSc) (van den Hoogen et al., 2013). The diffuse cutaneous form is characterized by dermal thickening and fibrosis proximal to the elbows and ankles and a higher risk of internal organ involvement compared to other forms. In limited cutaneous SSc, skin involvement is confined to the face, hands, fect, and/or forearms. In sine SSc, there is no skin involvement, but the condition is associated with characteristic scleroderma-associated autoantibodies and internal organ involvement (LeRoy et al., 1988; Wigley et al., 1994). Systemic sclerosis is a rare disease. Incidence rates and prevalence estimates are similar for Europe, the United States, Australia, and Argentina, suggesting a prevalence of 150-300 cases per million, with a lower prevalence noted in Scandinavia, Japan, the United Kingdom, Taiwan, and India (Barnes, et al., 2012). SSc is associated with substantial morbidity, reduced survival, and poor quality of life (QOL) (Medsger, et al., 1994; Wigley et al., 1994; Mayes, et al., 2003; Barnes, et al., 2012; Denton, et al., 2017).

Digital ischemic episodes (Raynaud's Phenomenon) are the most common manifestation of vascular abnormalities in SSc and a universal feature of SSc disease, affecting more than 95% of patients. Digital ischemic episodes were first described by Maurice Raynaud in 1862 when he recognized that some people who were exposed to cold temperature or emotional stress, had transient digital ischemia. The term “Raynaud's Phenomenon” (RP) is used to describe these digital ischemic episodes. RP affects 3-5% of people in the United States (US), mostly women, and is categorized in primary and/or secondary forms (Maundrell et al. Raynaud's Phenomenon; A Guide to Pathogenesis and Treatment. New York, NY Springer; 2015:21-35; Pope et al. BMJ Clin Evid. 2013:2013: 1119). Primary RP is characterized by the absence of an underlying condition or disorder, is responsible for most cases of RP (80-90%) (Maundrell 2015; Pope 2013) and typically develops at an early age, usually before the age of 30 (Levien et al. Vasc Health Risk Manag. 2010; 6: 167-177; Garner et al. BMJ Open. 2015:5(3):e006389). Primary RP typically presents as symmetric attacks and is not associated with severe sequelae. Secondary RP develops in association with an underlying disease or condition-usually a connective tissue disease, such as SSc—and typically begins after the age of 30 (Levien 2010). RP secondary to SSc is associated with significant disability, pain, and psychological impact (Merkel et al. Arthritis & Rheumatism. 2002:46(9):2410-20). In addition to pain, annoyance, and functional disability caused by RP attacks, many patients with SSc report that they change their daily routine to accommodate their RP and may have significant anxiety associated with their disease, often expressing fears concerning digital ulcers and potential autoamputation (Merkel 2002).

RP represents vasoconstriction of the digital arteries, precapillary arterioles and cutaneous arteriovenous shunts in response to cold or stress. In SSc, RP is associated with obliterative vasculopathy and structural changes and progress from biphasic to triphasic color changes of the fingers, toes, and other peripheral tissue without or with symptoms to digital ischemic ulcers and critical digital ischemia and to gangrene over time (Young et al., 2016). RP is also associated with recurrent painful attacks significant disability and psychological impact in patients with SSc (Merkel et al., 2002; Hummers et al., 2003).

RP in systemic sclerosis results from both functional and structural vascular abnormalities (Abraham and Steen, 2015). The structural component is twofold; the first marker is intimal proliferation and fibrosis, causing significant compromise of the vessel lumen. The resultant endothelial damage leads to the upregulation of vasoconstrictive mediators, such as endothelin-1 (Kahalch, 1991), while simultaneously lowering the levels of vasodilatory molecules, such as nitric oxide (NO) (Freedman et al., 1999) and prostacyclin (Abraham, et al., 2015). The functional component results from frequent vasospasm, contributed by increased sympathetic activation, which over time may lead to progressive tissue ischemia and the formation of oxygen-free radicals, perpetuating this cycle (Abraham, et al., 2015). Small blood vessels in affected tissues from patients with SSc show perivascular cellular infiltration by activated T-lymphocytes, similar to that seen in affected skin and internal organs (Kahaleh, 1991). The endothelial damage underlying the vasculopathy in SSc is also associated with platelet activation (Matucci-Cerinic et al., 2013). Digital ischemic ulcers (DIUs) are a frequent external manifestation of vasculopathy in SSc (Nihtyanova et al., 2008). DIUs are denuded areas of tissue that occur either at distal aspects of digits or over bony prominences with well-demarcated borders, involving loss of both the dermis and epidermis (Baron et al., 2014). Repeated bouts of RP lead to prolonged digital ischemia that may progress to DIU or extreme critical digital ischemia with gangrene (Silva et al., 2015). DIUs occur in up to 58% of patients with limited or diffuse SSc and often occur early in the disease course (Ferri et al., 2002; Walker et al., 2007; Matucci-Cerinic et al., 2016). A 30% annual incidence of SSc DIU has been reported in the literature. Of those patients who experience a DIU, more than half have persistent or recurrent DIUs for at least 6 months (Steen et al., 2009; Matucci-Cerinic et al., 2016). Several studies have shown that the lesions are painful, heal slowly, lead to substantial functional disability, and are associated with complications such as scarring, loss of distal tissue, infection, gangrene, amputation leading to reduced QOL, an increased frequency of hospitalization, and decreased survival (Chung and Fiorentino, 2006; Hachulla et al., 2007; Mouthon et al., 2010; Matucci-Cerinic et al., 2016). Several factors are implicated in the pathogenesis of DIUs in SSc. These include (i) impaired afferent vasomotion (highlighted by the intimal hyperplasia of arterioles), (ii) disrupted capillary and lymphatic microvasculature, (iii) leucocyte and platelet activation and adherence to injured endothelium, and (iv) haemorheological alterations typical of SSc. The disease is also characterized by insufficient angiogenesis and defective vasculogenesis, contributing further to tissue ischemia (Tsou et al., 2016).

### SUMMARY OF THE INVENTION

The present disclosure provides a pharmaceutical composition for injection, comprising iloprost or a pharmaceutically acceptable salt or stereoisomer thereof and a pharmaceutically acceptable carrier or excipient; wherein a single daily injection provides maximum serum concentration (Cmax) of iloprost is in the range of about 10 pg/mL to about 150 pg/mL and at least about 8 hours of iloprost-free period within 48 hours from each injection.

In embodiments of the pharmaceutical composition provided herein, the Cmax of iloprost is in the range of about 20 pg/mL to about 40 pg/mL. In embodiments, the Cmax of iloprost is in the range of about 40 pg/mL to about 60 pg/mL. In embodiments, the Cmax of iloprost is in the range of about 60 pg/mL to about 80 pg/mL. In embodiments, the Cmax of iloprost is in the range of about 80 pg/mL to about 130 pg/mL.

In embodiments of the pharmaceutical composition provided herein, the composition provides a serum concentration of iloprost at steady state (Css) is in the range of about 10 pg/mL to about 30 pg/mL. In embodiments, the composition provides a Css of iloprost is in the range of about 30 pg/mL to about 60 pg/mL. In embodiments, the composition provides a Css of iloprost is in the range of about 60 pg/mL to about 80 pg/mL. In embodiments, the composition provides a Css of iloprost is in the range of about 80 pg/mL to about 120 pg/mL. In embodiments, the composition provides a daily serum concentration of iloprost at steady state (Css) of about 6 hours to about 24 hours. In embodiments, the composition provides a daily serum concentration of iloprost at steady state (Css) for about 6 hours.

In embodiments of the pharmaceutical composition provided herein, the iloprost-free period is a duration in which iloprost is undetectable in the subject's plasma.

In embodiments of the pharmaceutical composition provided herein, the single daily injection does not result in daily accumulation of iloprost in the subject.

In embodiments of the pharmaceutical composition provided herein, the composition provides an area under the plasma concentration versus time curve from time of dosing to the last time point with measurable concentration serum concentration (AUC₀₋ₗₐₛₜ) of iloprost in the range of about 100 pg·hr/mL to about 200 pg·hr/mL. In embodiments, the composition provides an AUC₀₋ₗₐₛₜ of iloprost is in the range of about 200 pg·hr/mL to about 350 pg·hr/mL. In embodiments, the composition provides an AUC₀₋ₗₐₛₜ of iloprost is in the range of about 350 pg·hr/mL to about 500 pg hr/mL. In embodiments, the composition provides an AUC₀₋ₗₐₛₜ of iloprost is in the range of about 500 pg·hr/mL to about 700 pg·hr/mL.

In embodiments of the pharmaceutical composition provided herein, the injection is a subcutaneous injection.

In embodiments of the pharmaceutical composition provided herein, the single daily dose has a volume of less than about 1.0 mL.

In embodiments of the pharmaceutical composition provided herein, the single daily dose comprises about 100 ng iloprost or a pharmaceutically acceptable salt or stereoisomer thereof per weight of the subject (kg) to about 200 ng/kg. In embodiments, the single daily dose comprises iloprost or a pharmaceutically acceptable salt or stereoisomer thereof in about 200 ng/kg to about 400 ng/kg. In embodiments, the single daily dose comprises iloprost or a pharmaceutically acceptable salt or stereoisomer thereof in about 400 ng/kg to about 600 ng/kg. In embodiments, the single daily dose comprises iloprost or a pharmaceutically acceptable salt thereof or stereoisomer in about 600 ng/kg to about 800 ng/kg.

In embodiments of the pharmaceutical composition provided herein, the single daily dose comprises iloprost or a pharmaceutically acceptable salt or stereoisomer thereof in about 0.01% to about 20% by weight of the composition.

In embodiments of the pharmaceutical composition provided herein, the composition is bioequivalent to iloprost continuous infusion over 6 hours each day for 5 consecutive days at 0.5 to 2.0 ng iloprost/kg body weight/min.

In embodiments of the pharmaceutical composition provided herein, the composition is for use in combination with one or more days of treatment with iloprost continuous infusion over 6 hours/day at 0.5 to 2.0 ng iloprost/kg body weight/min. In embodiments, the composition is for use in combination with one day of treatment with iloprost continuous infusion over 6 hours/day at 0.5 to 2.0 ng iloprost/kg body weight/min. In embodiments, the composition is administered after the treatment with iloprost continuous infusion over 6 hours/day at 0.5 to 2.0 ng iloprost/kg body weight/min, wherein the administration of the composition is on the same day or on a different day than the treatment with iloprost continuous infusion. In embodiments, the composition is administered starting the day after the treatment with iloprost continuous infusion over 6 hours/day at 0.5 to 2.0 ng iloprost/kg body weight/min.

The present disclosure also provides a sustained release pharmaceutical composition for injection, comprising iloprost or a pharmaceutically acceptable salt or stereoisomer thereof and a pharmaceutically acceptable carrier or excipient; wherein the composition releases iloprost over a period of about 5 days to about 7 days.

In embodiments of the pharmaceutical composition provided herein, the composition provides a serum concentration of iloprost at steady state (Css) in the range of about 10 pg/mL to about 50 pg/mL. In embodiments, the composition provides a serum concentration of iloprost at steady state (Css) in the range of about 20 pg/mL to about 40 pg/mL.

In embodiments of the pharmaceutical composition provided herein, a single dose of the composition comprises about 5.0 mcg iloprost or a pharmaceutically acceptable salt or stereoisomer thereof per weight of the subject (kg) to about 10.0 mcg/kg.

In embodiments of the pharmaceutical composition provided herein, the injection is a subcutaneous injection.

In embodiments of the pharmaceutical composition provided herein, a single daily dose comprises iloprost or a pharmaceutically acceptable salt or stereoisomer thereof in about 0.01% to about 20% by weight of the composition.

In embodiments of any one of the pharmaceutical compositions provided herein, the composition comprises a phospholipid. In embodiments, the phospholipid is lecithin, phosphatidylcholine, phosphotidylethanolamine, phosphotidylserine, phosphatidylinositol, phosphoglyceride, phosphoglycerol, phospholipid, sphingosine, ganglioside, phytosphingosine, diacylglycerol, phosphocholine, phosphoethanolamine, hosphotidylserine, lysophospholipid, pegylated phospholipid, mixed chain phospholipid, or a combination thereof. In embodiments, the lecithin is soy lecithin, egg lecithin, or a combination thereof. In embodiments, the composition comprises a phospholipid in about 20% to about 80% by weight of the composition.

In embodiments of any one of the pharmaceutical compositions provided herein, the composition comprises an oil. In embodiments, the oil is synthetic oil, a vegetable oil, sesame oil, a medium chain oil, silicone oil, ethyl oleate, fatty acid, vitamin E, vitamin E succinate, cholesterol, triglyceride oil, or a mixture thereof.

In embodiments of any one of the pharmaceutical composition provided herein, the pharmaceutically acceptable excipient is sucrose, dextrose, lactose, glucose, trehalose, maltose, mannitol, sorbitol, glycerol, amylose, starch, amylopectin, triglycerides, fatty acids, carbohydrates, or a mixture thereof. In embodiments, the pharmaceutically acceptable excipient is an acidifying agent, an alkalizing agent, a pH buffering agent, a metal ion chelator, an antioxidant, a preservative, a tonicity/osmotic pressure modifier, a condensing agent, a solubilizing agent, or a mixture thereof.

In embodiments of any one of the pharmaceutical compositions provided herein, the pharmaceutically acceptable carrier is ethanol, propylene glycol, glycerol, sorbitol, polyethylene glycol, silicone oil, glycofurol, ethyl oleate, or a mixture thereof.

In embodiments of any one of the pharmaceutical compositions provided herein, the composition is an injectable gel composition.

In embodiments of any one of the pharmaceutical compositions provided herein, the composition is a nanoemulsion.

In embodiments of any one of the pharmaceutical compositions provided herein, the composition comprises a liposome. In embodiments, the liposome comprises phosphatidyl choline, phosphatidyl glycerol, phosphatidyl serine, phosphatidic acid, cholesterol, cholesterol derivatives, phosphatidyl ethanolamines, neutral lipids, cationic lipids, or combinations thereof. In embodiments, the liposome comprises dimyristoylphosphatidyl choline (DMPC), distearoylphosphatidyl choline (DSPC), dipalmitoyl phosphatidyl choline (DPPC), dimyristoylphosphatidyl glycerol (DMPG), dipalmitoylphosphatidyl glycerol (DPPG), distearoylphosphatidyl glycerol (DSPG), dimyristoylphosphatidyl serine (DMPS), dipalmitoylphosphatidyl serine (DPPS), distearoylphosphatidyl serine (DSPS), dimyristoylphosphatidic acid (DMPA), dipalmitoylphosphatidic acid (DPPA), cholesterol hemisuccinate (CHEMS), hydrogenated soy bean phosphatidyl choline (HSPC), distearoyl phosphatidyl choline (DSPC), hydrogenated egg phosphatidyl choline (HEPC), or combinations thereof. In embodiments, the liposome comprises a lipid, a phospholipid, a cholesterol, or a combination thereof.

In embodiments of any one of the pharmaceutical compositions provided herein, the iloprost or a pharmaceutically acceptable salt or stereoisomer thereof is entrapped or encapsulated in the liposome.

In embodiments of any one of the pharmaceutical compositions provided herein, the liposome has a mean particle diameter in the range of about 10 nm to about 1,000 nm. In embodiments, the liposome has a mean particle diameter in the range of about 10 nm to about 300 nm.

In embodiments of any one of the pharmaceutical compositions provided herein, the composition comprises microspheres. In embodiments, the microspheres are polymer microspheres.

In embodiments of any one of the pharmaceutical composition provided herein, the microspheres comprise polylactide polymer, sodium acrylate polymer, acrylamide polymer, acrylamide derivative polymer or copolymer, sodium acrylate and vinyl alcohol copolymer, a vinyl acetate and acrylic acid ester copolymer, a vinyl acetate and methyl maleate copolymer, and a isobutylene-maleic anhydride crosslinked copolymer, polylactic acid (PLA), polyglycolic acid (PGA), a copolymer of PLA and PGA, poly-L-lactide (PLLA), poly-D,L-lactide (PDLA), poly-capralactone (PCL), poly(D,L-lactide-co-glycolide), poly(3-hydroxy-butyrate), polylactone, polyanhydride, poly(hydroxy-butyrate)-co-(hydroxy-valerate), polypropylene-glucose, poly(lactic acid)-polyglycol, and poly(hydroxyacetic acid)-polyglycol, or a combination thereof.

In embodiments of any one of the pharmaceutical compositions provided herein, the microspheres comprise a biodegradable wall.

In embodiments of any one of the pharmaceutical compositions provided herein, the microspheres have a mean diameter of about 10 μm to about 300 μm.

In embodiments of any one of the pharmaceutical compositions provided herein, the iloprost or a pharmaceutically acceptable salt or stereoisomer thereof is entrapped or encapsulated in the microsphere.

In embodiments of any one of the pharmaceutical compositions provided herein, the composition comprises a gel. In embodiments, the gel is a phospholipid gel. In embodiments, the gel is a polymer gel.

In embodiments of any one of the pharmaceutical composition provided herein, the gel comprises xanthan gum, deacylated xanthan gum, a carboxymethyl ether, propylene glycol ester, homo polysaccharide gum, galactomannan gum, hydroxypropyl cellulose, hydroxypropylmethyl cellulose, polylactide polymer, sodium acrylate polymer, acrylamide polymer, acrylamide derivative polymer or copolymer, sodium acrylate and vinyl alcohol copolymer, a vinyl acetate and acrylic acid ester copolymer, a vinyl acetate and methyl maleate copolymer, and a isobutylene-maleic anhydride crosslinked copolymer, polylactic acid (PLA), polyglycolic acid (PGA), a copolymer of PLA and PGA, poly-L-lactide (PLLA), poly-D,L-lactide (PDLA), poly-capralactone (PCL), poly(D,L-lactide-co-glycolide), poly(3-hydroxy-butyrate), polylactone, polyanhydride, poly(hydroxy-butyrate)-co-(hydroxy-valerate), polypropylene-glucose, poly(lactic acid)-polyglycol, and poly(hydroxyacetic acid)-polyglycol, or a combination thereof.

In embodiments of any one of the pharmaceutical compositions provided herein, the iloprost or a pharmaceutically acceptable salt or stereoisomer thereof is entrapped or encapsulated in the gel.

In embodiments of any one of the pharmaceutical compositions provided herein, the composition comprises a peptide. In embodiments, the peptide is non-covalently bonded with the iloprost or a pharmaceutically acceptable salt or stereoisomer thereof. In embodiments, the peptide comprises a primarily acidic peptide fragment and a primarily basic peptide fragment, with a neutral peptide fragment in between the acidic and basic fragments. In embodiments, the ratio of iloprost or a pharmaceutically acceptable salt or stereoisomer thereof to the peptide is in the range of about 1:2 to about 1:10. In embodiments, the composition comprising a peptide further comprises a solvent selected from water, tetrahydrofuran, dimethylformamide, dimethylsulfoxide or acetonitrile, or a combination thereof.

The present disclosure also provides a method of treating symptomatic Raynaud's Phenomenon (RP) in a subject with systemic sclerosis (SSc), comprising administering any one of the pharmaceutical compositions comprising iloprost or a pharmaceutically acceptable salt or stereoisomer thereof, as described herein. In embodiments, the method reduces the frequency of symptomatic RP episodes. In embodiments, the method reduces the average duration of symptomatic RP episodes. In embodiments, the method reduces the severity of symptomatic RP episodes. In embodiments, the method increases the number of days without symptomatic RP attacks. In embodiments, the method reduces the pain in the subject's fingers associated with symptomatic RP episodes. In embodiments, the method reduces the severity of numbness in the subject's fingers associated with symptomatic RP episodes. In embodiments, the method reduces the severity of tingling in the subject's fingers associated with symptomatic RP episodes. In embodiments, the method reduces the severity of discomfort in the subject's fingers associated with symptomatic RP episodes.

The present disclosure also provides a method of preventing the development of digital ischemia in a subject with systemic sclerosis (SSc), comprising administering any one of the pharmaceutical compositions comprising iloprost or a pharmaceutically acceptable salt or stereoisomer thereof, as described herein. In embodiments, the digital ischemia is critical digital ischemia.

The present disclosure also provides a method of preventing the development of RP episodes in a subject with systemic sclerosis (SSc), comprising administering any one of the pharmaceutical compositions comprising iloprost or a pharmaceutically acceptable salt or stereoisomer thereof, as described herein.

The present disclosure also provides a method of treating and preventing the development of digital infection, digital ischemic lesion, digital ischemic ulcer, or gangrene in a subject with systemic sclerosis (SSc), comprising administering any one of the pharmaceutical composition comprising iloprost or a pharmaceutically acceptable salt or stereoisomer thereof, as described herein.

In embodiments of any one of the methods provided herein, the pharmaceutical composition comprising iloprost or a pharmaceutically acceptable salt or stereoisomer thereof is administered in combination with one or more days of treatment with iloprost continuous infusion over 6 hours/day at 0.5 to 2.0 ng iloprost/kg body weight/min. In embodiments, the pharmaceutical composition is administered in combination with one day of treatment with iloprost continuous infusion over 6 hours/day at 0.5 to 2.0 ng iloprost/kg body weight/min. In embodiments, the pharmaceutical composition is administered after the treatment with iloprost continuous infusion over 6 hours/day at 0.5 to 2.0 ng iloprost/kg body weight/min, wherein the administration of the pharmaceutical composition is on the same day or on a different day than the treatment with iloprost continuous infusion. In embodiments, the pharmaceutical composition is administered starting the day after the treatment with iloprost continuous infusion over 6 hours/day at 0.5 to 2.0 ng iloprost/kg body weight/min.

### BRIEF DESCRIPTION OF THE DRAWINGS

FIG. 1 shows a Schematic of iloprost 2-compartment PK model.

FIG. 2A shows the mean simulated PK profile of iloprost from a 2.0 ng/kg/min over 6 hours daily infusion over 5 days showing no accumulation. FIG. 2B shows the mean simulated PK profile at Day 5 of iloprost from a 2.0 ng/kg/min over 6 hours daily infusion over 5 days showing no accumulation.

FIG. 3A shows the mean simulated PK profile of iloprost from a 0.5 ng/kg/min over 6 hours daily infusion over 5 days showing no accumulation. FIG. 3B shows the mean simulated PK profile at Day 5 of iloprost from a 0.5 ng/kg/min over 6 hours daily infusion over 5 days showing no accumulation.

FIG. 4 shows mean (±SD) iloprost plasma concentration-time profiles following a single subcutaneous injection of 21.4, 19.2, and 19.3 μg/animal iloprost long-acting formulations, Formulations 1, 2, and 3, respectively, to male rats.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “an IP receptor agonist” refers to one or more IP receptor agonists or at least one IP receptor agonist. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as an acid, with an inorganic or organic base to form a salt. Organic base includes, but are not limited to, monoethanolamine, diethanolamine, triethanolamine, trometamol and meglumine. Those skilled in the art will further recognize that base addition salts may be prepared by reaction of the compounds with the appropriate inorganic or organic base via any of a number of known methods.

The term “nanodispersion” as used herein includes a suspension or emulsion where an average particle diameter is from about 5 nm to about 500 nm. In embodiments, nanodispersion has an average particle diameter from about 5 nm to about 200 nm. Nanodispersion can be further characterized as a “nanoemulsion” or a “nanosuspension”. As used herein, the term “emulsion” includes a mixture of immiscible phases which forms small droplets which are suspended or dispersed in the mixture. In embodiments, an emulsion includes a mixture of immiscible hydrophobic phase and aqueous phase formed, wherein the hydrophobic phase is formed into small droplets which are dispersed throughout the aqueous phase.

The term “nanoemulsion” as used herein includes an emulsion where an average particle diameter is from about 5 nm to about 500 nm. In embodiments, nanoemulsion has an average particle diameter from about 5 nm to about 200 nm. In embodiments, oil is present in a nanoemulsion.

The term “nanosuspension” as used herein includes an emulsion where an average particle diameter is from about 5 nm to about 500 nm. In embodiments, nanosuspension has an average particle diameter from about 5 nm to about 200 nm. In embodiments, oil is not present in a nanosuspension.

The term “single-phase” as used herein refers to a composition that is substantially uniform in its contents. For example, a single-phase gel composition comprising iloprost or a pharmaceutically acceptable salt or stereoisomer thereof, has a uniform content such that iloprost or a pharmaceutically acceptable salt or stereoisomer thereof is uniformly distributed throughout the gel composition.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compound of the invention, or their pharmaceutically acceptable salts contain asymmetric centers and can thus give rise to enantiomers, diastereomers, and other stereoisomeric forms that can be defined, in terms of absolute stereochemistry, as (R) or (S). The present disclosure is meant to include all such possible isomers, as well as their racemic and optically pure forms whether or not they are specifically depicted herein. Stereoisomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). The compound described herein also contains an olefinic double bond, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes diastereomers.

### Pharmaceutically Active Ingredient

The present disclosure relates to injection formulation of iloprost or a pharmaceutically acceptable salt thereof or a stereoisomer thereof. Iloprost has the following structure and can also be identified as (5E)-5-[(3aS,4R,5R,6aS)-5-hydroxy-4-[(E,3S)-3-hydroxy-4-methyloct-1-en-6-ynyl]-3,3a,4,5,6,6a-hexahydro-1H-pentalen-2-ylidene]pentanoic acid.

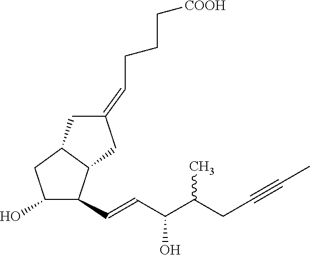 [SMILES: CC#CCC(C)[C@H](O)/C=C/[C@@H]1[C@H]2C/C(=C/CCCC(=O)O)C[C@H]2C[C@H]1O]

The present disclosure also relates to method of use of the injection formulation of iloprost or a pharmaceutically acceptable salt or stereoisomer thereof. In embodiments, iloprost or a pharmaceutically acceptable salt or stereoisomer thereof is useful for treating systemic sclerosis (SSc) with symptomatic Raynaud's Phenomenon (RP) and/or with digital ulcers.

### Formulation for Injection

The present disclosure relates to pharmaceutical compositions comprising iloprost or a pharmaceutically acceptable salt thereof or a stereoisomer thereof and a pharmaceutically acceptable carrier or excipient. In embodiments, the pharmaceutical composition is an injectable composition. In embodiments, the composition is administered by intravenous, intramuscular, or subcutaneous injection. In embodiments, the composition is administered by intramuscular, or subcutaneous injection. In embodiments, the composition is not administered by continuous injection or infusion.

In embodiments, the pharmaceutical compositions of the present disclosure can be administered by a medical professional or the composition can be self-administered by the patient.

In embodiments, the present disclosure relates to sustained or controlled release pharmaceutical compositions comprising iloprost or a pharmaceutically acceptable salt or a stereoisomer thereof and a pharmaceutically acceptable carrier or excipient. In many cases of developing controlled release formulations, the drug is highly soluble in the matrix and may leave the matrix too rapidly, leading to drug toxicities. However, iloprost is a poorly soluble compound, which makes it a good candidate for formulating into various types of control release formulations across multiple drug delivery systems.

In embodiments, the dose and the release kinetics of iloprost in a controlled release formulation can be estimated based on iloprost clearance, desired serum concentration of iloprost at steady state (Css), and desired duration of iloprost exposure. The transport of a drug within the matrix or deport is an important factor for drug release kinetics and it is related to the diffusivity and the solubility.

Many unstable drugs, like iloprost, can be unstable in controlled release formulations. However, pharmaceutically acceptable excipients such as a pH adjusting agent (e.g., acidifying agent, alkalizing agent, or pH buffering agent) or a stabilizer can improve stability of the drug within the formulation. Also, different techniques for mixing the drug with the matrix during formulating process can impact the stability of the drug within the resulting formulation. During manufacturing a nitrogen overlay and equipment purging can be used to reduce the oxidative degradation of iloprost.

The present disclosure relates to a pharmaceutical composition for injection, comprising iloprost or a pharmaceutically acceptable salt or stereoisomer thereof and a pharmaceutically acceptable carrier or excipient; wherein a single daily injection provides maximum serum concentration (Cmax) of iloprost is in the range of about 10 pg/mL to about 150 pg/mL and at least about 8 hours of iloprost-free period within 48 hours from each injection. In embodiments, a pharmaceutical composition for injection, comprising iloprost or a pharmaceutically acceptable salt or stereoisomer thereof and a pharmaceutically acceptable carrier or excipient; wherein a single daily injection provides maximum serum concentration (Cmax) of iloprost is in the range of about 10 pg/mL to about 150 pg/mL and at least about 8 hours of iloprost-free period within 36 hours from each injection. In embodiments, a single daily injection of the pharmaceutical composition provides at least about 8 hours of iloprost-free period within 48 hours, within 47 hours, within 46 hours, within 45 hours, within 44 hours, within 43 hours, within 42 hours, within 41 hours, within 40 hours, within 39 hours, within 38 hours, within 37 hours, within 36 hours, within 35 hours, within 34 hours, within 33 hours, within 32 hours, within 31 hours, within 30 hours, within 29 hours, within 28 hours, within 27 hours, within 26 hours, within 25 hours, or within 24 hours from each injection.

The present disclosure relates to a pharmaceutical composition for injection, comprising iloprost or a pharmaceutically acceptable salt or stereoisomer thereof and a pharmaceutically acceptable carrier or excipient; wherein a single daily injection provides maximum serum concentration (Cmax) of iloprost is in the range of about 10 pg/mL to about 150 pg/mL and at least about 8 hours of iloprost-free period within 24 hours from each injection.

In embodiments of the pharmaceutical composition of the present disclosure, the composition provides a Cmax of iloprost is in the range of 10 pg/mL to about 130 pg/mL, including all values and subranges therebetween. In embodiments, the composition provides a Cmax of iloprost in the range of about 10 pg/mL to about 50 pg/mL, including all values therebetween. In embodiments, the composition provides a Cmax of iloprost is in the range of about 20 pg/mL to about 40 pg/mL, including all values therebetween. In embodiments, the composition provides a Cmax of iloprost of about 25 pg/mL or about 30 pg/mL. In embodiments, the composition provides a Cmax of iloprost of about 15 pg/mL, about 16 pg/mL, about 17 pg/mL, about 18 pg/mL, about 19 pg/mL, about 20 pg/mL, about 21 pg/mL, about 22 pg/mL, about 23 pg/mL, about 24 pg/mL, about 25 pg/mL, about 26 pg/mL, about 27 pg/mL, about 28 pg/mL, about 29 pg/mL, about 30 pg/mL, about 31 pg/mL, about 32 pg/mL, about 33 pg/mL, about 34 pg/mL, about 35 pg/mL, about 36 pg/mL, about 37 pg/mL, about 38 pg/mL, about 39 pg/mL, or about 40 pg/mL.

In embodiments of the pharmaceutical composition of the present disclosure, the composition provides a Cmax of iloprost in the range of about 40 pg/mL to about 70 pg/mL, including all values therebetween. In embodiments, the composition provides a Cmax of iloprost in the range of about 40 pg/mL to about 60 pg/mL, including all values therebetween. In embodiments, the composition provides a Cmax of iloprost of about 50 pg/mL. In embodiments, the composition provides a Cmax of iloprost of about 40 pg/mL, about 41 pg/mL, about 42 pg/mL, about 43 pg/mL, about 44 pg/mL, about 45 pg/mL, about 46 pg/mL, about 47 pg/mL, about 48 pg/mL, about 49 pg/mL, about 50 pg/mL, about 51 pg/mL, about 52 pg/mL, about 53 pg/mL, about 54 pg/mL, about 55 pg/mL, about 56 pg/mL, about 57 pg/mL, about 58 pg/mL, about 59 pg/mL, or about 60 pg/mL.

In embodiments of the pharmaceutical composition of the present disclosure, the composition provides a Cmax of iloprost in the range of about 70 pg/mL to about 110 pg/mL, including all values therebetween. In embodiments, the composition provides a Cmax of iloprost in the range of about 60 pg/mL to about 80 pg/mL, including all values therebetween. In embodiments, the composition provides a Cmax of iloprost of about 70 pg/mL or about 75 pg/mL. In embodiments, the composition provides a Cmax of iloprost of about 60 pg/mL, about 61 pg/mL, about 62 pg/mL, about 63 pg/mL, about 64 pg/mL, about 65 pg/mL, about 66 pg/mL, about 67 pg/mL, about 68 pg/mL, about 69 pg/mL, about 70 pg/mL, about 71 pg/mL, about 72 pg/mL, about 73 pg/mL, about 74 pg/mL, about 75 pg/mL, about 76 pg/mL, about 77 pg/mL, about 78 pg/mL, about 79 pg/mL, about 80 pg/mL, about 81 pg/mL, about 82 pg/mL, about 83 pg/mL, about 84 pg/mL, or about 85 pg/mL.

In embodiments of the pharmaceutical composition of the present disclosure, the composition provides a Cmax of iloprost in the range of about 80 pg/mL to about 150 pg/mL, including all values and subranges therebetween. In embodiments, the composition provides a Cmax of iloprost in the range of about 80 pg/mL to about 130 pg/mL, including all values therebetween. In embodiments, the composition provides a Cmax of iloprost in the range of about 80 pg/mL to about 120 pg/mL, including all values therebetween. In embodiments, the composition provides a Cmax of iloprost of about 100 pg/mL. In embodiments, the composition provides a Cmax of iloprost of about 80 pg/mL, about 81 pg/mL, about 82 pg/mL, about 83 pg/mL, about 84 pg/mL, about 85 pg/mL, about 86 pg/mL, about 87 pg/mL, about 88 pg/mL, about 89 pg/mL, about 90 pg/mL, about 91 pg/mL, about 92 pg/mL, about 93 pg/mL, about 94 pg/mL, about 95 pg/mL, about 96 pg/mL, about 97 pg/mL, about 98 pg/mL, about 99 pg/mL, about 100 pg/mL, about 101 pg/mL, about 102 pg/mL, about 103 pg/mL, about 104 pg/mL, about 105 pg/mL, about 106 pg/mL, about 107 pg/mL, about 108 pg/mL, about 109 pg/mL, about 110 pg/mL, about 111 pg/mL, about 112 pg/mL, about 113 pg/mL, about 114 pg/mL, about 115 pg/mL, about 116 pg/mL, about 117 pg/mL, about 118 pg/mL, about 119 pg/mL, about 120 pg/mL, about 121 pg/mL, about 122 pg/mL, about 123 pg/mL, about 124 pg/mL, about 125 pg/mL, about 126 pg/mL, about 127 pg/mL, about 128 pg/mL, about 129 pg/mL, or about 130 pg/mL.

In embodiments of the pharmaceutical composition of the present disclosure, the composition provides a serum concentration of iloprost at steady state (Css) in the range of 10 pg/mL to about 120 pg/mL, including all values and subranges therebetween. In embodiments, the composition provides a Css of iloprost in the range of about 10 pg/mL to about 30 pg/mL, including all values therebetween. In embodiments, the composition provides a Css of iloprost in the range of about 20 pg/mL to about 30 pg/mL, including all values therebetween. In embodiments, the composition provides a Css of iloprost of about 24 pg/mL. In embodiments, the composition provides a Css of iloprost of about 10 pg/mL, about 11 pg/mL, about 12 pg/mL, about 13 pg/mL, about 14 pg/mL, about 15 pg/mL, about 16 pg/mL, about 17 pg/mL, about 18 pg/mL, about 19 pg/mL, about 20 pg/mL, about 21 pg/mL, about 22 pg/mL, about 23 pg/mL, about 24 pg/mL, about 25 pg/mL, about 26 pg/mL, about 27 pg/mL, about 28 pg/mL, about 29 pg/mL, or about 30 pg/mL.

In embodiments of the pharmaceutical composition of the present disclosure, the composition provides a Css of iloprost in the range of about 30 pg/mL to about 60 pg/mL, including all values and subranges therebetween. In embodiments, the composition provides a Css of iloprost in the range of about 40 pg/mL to about 60 pg/mL, including all values therebetween. In embodiments, the composition provides a Css of iloprost in the range of about 40 pg/mL to about 55 pg/mL, including all values therebetween. In embodiments, the composition provides a Css of iloprost of about 49 pg/mL. In embodiments, the composition provides a Css of iloprost of about 30 pg/mL, about 31 pg/mL, about 32 pg/mL, about 33 pg/mL, about 34 pg/mL, about 35 pg/mL, about 36 pg/mL, about 37 pg/mL, about 38 pg/mL, about 39 pg/mL, about 40 pg/mL, about 41 pg/mL, about 42 pg/mL, about 43 pg/mL, about 44 pg/mL, about 45 pg/mL, about 46 pg/mL, about 47 pg/mL, about 48 pg/mL, about 49 pg/mL, about 50 pg/mL, about 51 pg/mL, about 52 pg/mL, about 53 pg/mL, about 54 pg/mL, about 55 pg/mL, about 56 pg/mL, about 57 pg/mL, about 58 pg/mL, about 59 pg/mL, or about 60 pg/mL.

In embodiments of the pharmaceutical composition of the present disclosure, the composition provides a Css of iloprost in the range of about 60 pg/mL to about 80 pg/mL, including all values and subranges therebetween. In embodiments, the composition provides a Css of iloprost in the range of about 65 pg/mL to about 80 pg/mL, including all values therebetween. In embodiments, the composition provides a Css of iloprost of about 73 pg/mL. In embodiments, the composition provides a Css of iloprost of about 60 pg/mL, about 61 pg/mL, about 62 pg/mL, about 63 pg/mL, about 64 pg/mL, about 65 pg/mL, about 66 pg/mL, about 67 pg/mL, about 68 pg/mL, about 69 pg/mL, about 70 pg/mL, about 71 pg/mL, about 72 pg/mL, about 73 pg/mL, about 74 pg/mL, about 75 pg/mL, about 76 pg/mL, about 77 pg/mL, about 78 pg/mL, about 79 pg/mL, or about 80 pg/mL.

In embodiments of the pharmaceutical composition of the present disclosure, the composition provides a Css of iloprost in the range of about 80 pg/mL to about 120 pg/mL, including all values and subranges therebetween. In embodiments, the composition provides a Css of iloprost in the range of about 85 pg/mL to about 110 pg/mL, including all values therebetween. In embodiments, the composition provides a Css of iloprost of about 97 pg/mL. In embodiments, the composition provides a Css of iloprost of about 80 pg/mL, about 81 pg/mL, about 82 pg/mL, about 83 pg/mL, about 84 pg/mL, about 85 pg/mL, about 86 pg/mL, about 87 pg/mL, about 88 pg/mL, about 89 pg/mL, about 90 pg/mL, about 91 pg/mL, about 92 pg/mL, about 93 pg/mL, about 94 pg/mL, about 95 pg/mL, about 96 pg/mL, about 97 pg/mL, about 98 pg/mL, about 99 pg/mL, about 100 pg/mL, about 101 pg/mL, about 102 pg/mL, about 103 pg/mL, about 104 pg/mL, about 105 pg/mL, about 106 pg/mL, about 107 pg/mL, about 108 pg/mL, about 109 pg/mL, about 110 pg/mL, about 111 pg/mL, about 112 pg/mL, about 113 pg/mL, about 114 pg/mL, about 115 pg/mL, about 116 pg/mL, about 117 pg/mL, about 118 pg/mL, about 119 pg/mL, or about 120 pg/mL.

In embodiments of the pharmaceutical composition of the present disclosure, the composition provides a daily Css of iloprost for about 4 hours to about 24 hours, including all values and subranges therebetween. In embodiments of the pharmaceutical composition of the present disclosure, the composition provides a daily Css of iloprost for about 4 hours to about 16 hours, including all values and subranges therebetween. In embodiments, the composition provides a daily Css of iloprost for about 6 hours to about 18 hours, including all values and subranges therebetween. In embodiments, the composition provides a daily Css of iloprost for about 6 hours to about 12 hours, including all values and subranges therebetween. In embodiments, the composition provides a daily Css of iloprost for at least about 6 hours. In embodiments, the composition provides a daily Css of iloprost for about 12 hours or less. In embodiments, the composition provides a daily Css of iloprost for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, including all values therebetween.

In embodiments of the pharmaceutical composition of the present disclosure, the composition provides an area under the plasma concentration versus time curve from time of dosing to the last time point with measurable concentration serum concentration (AUC₀₋ₗₐₛₜ) of iloprost in the range of about 20 pg·hr/mL to about 1,500 pg·hr/mL, including all values and subranges therebetween. In embodiments, the composition provides an AUC₀₋ₗₐₛₜ of iloprost in the range of about 100 pg·hr/mL to about 300 pg·hr/mL, including all values and subranges therebetween. In embodiments, the composition provides an AUC₀₋ₗₐₛₜ of iloprost in the range of about 100 pg·hr/mL to about 200 pg·hr/mL, including all values and subranges therebetween. In embodiments, the composition provides an AUC₀₋ₗₐₛₜ of iloprost in the range of about 150 pg·hr/mL to about 650 pg·hr/mL, including all values and subranges therebetween. In embodiments, the composition provides an AUC₀₋ₗₐₛₜ of iloprost in the range of about 150 pg·hr/mL to about 600 pg hr/mL, including all values and subranges therebetween. In embodiments, the composition provides an AUC₀₋ₗₐₛₜ of iloprost in the range of about 200 pg·hr/mL to about 400 pg hr/mL, including all values and subranges therebetween. In embodiments, the composition provides an AUC₀₋ₗₐₛₜ of iloprost in the range of about 200 pg·hr/mL to about 350 pg hr/mL, including all values and subranges therebetween. In embodiments, the composition provides an AUC₀₋ₗₐₛₜ of iloprost in the range of about 200 pg hr/mL to about 500 pg hr/mL, including all values and subranges therebetween. In embodiments, the composition provides an AUC₀₋ₗₐₛₜ of iloprost in the range of about 350 pg·hr/mL to about 500 pg hr/mL, including all values and subranges therebetween. In embodiments, the composition provides an AUC₀₋ₗₐₛₜ of iloprost in the range of about 350 pg·hr/mL to about 800 pg hr/mL, including all values and subranges therebetween. In embodiments, the composition provides an AUC₀₋ₗₐₛₜ of iloprost in the range of about 350 pg·hr/mL to about 600 pg hr/mL, including all values and subranges therebetween. In embodiments, the composition provides an AUC₀₋ₗₐₛₜ of iloprost is in the range of about 200 pg·hr/mL to about 700 pg·hr/mL. In embodiments, the composition provides an AUC₀₋ₗₐₛₜ of iloprost is in the range of about 350 pg·hr/mL to about 700 pg·hr/mL. In embodiments, the composition provides an AUC₀₋ₗₐₛₜ of iloprost in the range of about 500 pg hr/mL to about 700 pg·hr/mL, including all values and subranges therebetween.

In embodiments of the pharmaceutical composition of the present disclosure, the iloprost-free period is a duration in which iloprost is undetectable in the subject's plasma.

In embodiments of the pharmaceutical composition of the present disclosure, the composition given once daily does not result in daily accumulation of iloprost in the subject.

In embodiments of the pharmaceutical composition of the present disclosure, the composition is bioequivalent to iloprost continuous infusion over 6 hours each day for 5 consecutive days at 0.5 to 2.0 ng iloprost/kg body weight/min. In embodiments of the pharmaceutical composition of the present disclosure, the composition is bioequivalent to iloprost continuous infusion over 6 hours each day for 5 consecutive days at about 0.5 ng iloprost/kg body weight/min. In embodiments of the pharmaceutical composition of the present disclosure, the composition is bioequivalent to iloprost continuous infusion over 6 hours each day for 5 consecutive days at about 1.0 ng iloprost/kg body weight/min. In embodiments of the pharmaceutical composition of the present disclosure, the composition is bioequivalent to iloprost continuous infusion over 6 hours each day for 5 consecutive days at about 1.5 ng iloprost/kg body weight/min. In embodiments of the pharmaceutical composition of the present disclosure, the composition is bioequivalent to iloprost continuous infusion over 6 hours each day for 5 consecutive days at about 2.0 ng iloprost/kg body weight/min.

In embodiments of the pharmaceutical composition of the present disclosure, the injection is subcutaneous or intramuscular injection. In embodiments, the injection is subcutaneous injection.

In embodiments of the pharmaceutical composition of the present disclosure, the single daily dose or a unit dose has a volume of less than about 1 mL. In embodiments of the pharmaceutical composition of the present disclosure, the single daily dose or a unit dose has a volume of less than about 1.0 mL. In embodiments of the pharmaceutical composition of the present disclosure, the single daily dose or a unit dose has a volume of less than about 0.5 mL. In embodiments of the pharmaceutical composition of the present disclosure, the single daily dose or a unit dose has a volume of less than about 1.5 mL, less than about 1.4 mL, less than about 1.3 mL, less than about 1.2 mL, less than about 1.1 mL, less than about 1.0 mL, less than about 0.9 mL, less than about 0.8 mL, less than about 0.7 mL, less than about 0.6 mL, or less than about 0.5 mL.

In embodiments of the pharmaceutical composition of the present disclosure, the single daily dose or a unit dose comprises about 100 ng iloprost or a pharmaceutically acceptable salt or stereoisomer thereof per weight of the subject (kg) to about 2,000 ng/kg, including all values and subranges therebetween. In embodiments, the single daily dose or the unit dose comprises about 100 ng/kg to about 1,500 ng/kg iloprost or a pharmaceutically acceptable salt or stereoisomer thereof, including all values and subranges therebetween. In embodiments, the single daily dose or the unit dose comprises about 100 ng/kg to about 1,000 ng/kg iloprost or a pharmaceutically acceptable salt or stereoisomer thereof, including all values and subranges therebetween. In embodiments, the single daily dose or the unit dose comprises about 100 ng/kg to about 300 ng/kg iloprost or a pharmaceutically acceptable salt or stereoisomer thereof, including all values and subranges therebetween. In embodiments, the single daily dose or the unit dose comprises about 100 ng/kg to about 200 ng/kg iloprost or a pharmaceutically acceptable salt or stereoisomer thereof, including all values and subranges therebetween. In embodiments, the single daily dose or the unit dose comprises about 180 ng/kg iloprost or a pharmaceutically acceptable salt or stereoisomer thereof.

In embodiments, the single daily dose or the unit dose comprises about 200 ng/kg to about 500 ng/kg iloprost or a pharmaceutically acceptable salt or stereoisomer thereof, including all values and subranges therebetween. In embodiments, the single daily dose or the unit dose comprises about 200 ng/kg to about 400 ng/kg iloprost or a pharmaceutically acceptable salt or stereoisomer thereof, including all values and subranges therebetween. In embodiments, the single daily dose or the unit dose comprises about 360 ng/kg iloprost or a pharmaceutically acceptable salt or stereoisomer thereof.

In embodiments, the single daily dose or the unit dose comprises about 400 ng/kg to about 650 ng/kg iloprost or a pharmaceutically acceptable salt or stereoisomer thereof, including all values and subranges therebetween. In embodiments, the single daily dose or the unit dose comprises about 400 ng/kg to about 600 ng/kg iloprost or a pharmaceutically acceptable salt or stereoisomer thereof, including all values and subranges therebetween. In embodiments, the single daily dose or the unit dose comprises about 540 ng/kg iloprost or a pharmaceutically acceptable salt or stereoisomer thereof.

In embodiments, the single daily dose or the unit dose comprises about 600 ng/kg to about 1,000 ng/kg iloprost or a pharmaceutically acceptable salt or stereoisomer thereof, including all values and subranges therebetween. In embodiments, the single daily dose or the unit dose comprises about 600 ng/kg to about 800 ng/kg iloprost or a pharmaceutically acceptable salt or stereoisomer thereof, including all values and subranges therebetween. In embodiments, the single daily dose or the unit dose comprises about 720 ng/kg iloprost or a pharmaceutically acceptable salt or stereoisomer thereof.

In embodiments of the pharmaceutical composition of the present disclosure, the composition comprises iloprost or a pharmaceutically acceptable salt or stereoisomer thereof in about 0.01% to about 30% by weight of the composition. In embodiments, the composition comprises iloprost or a pharmaceutically acceptable salt or stereoisomer thereof in about 0.01% to about 20% by weight of the composition, including all values and subranges therebetween. In embodiments, the composition comprises iloprost or a pharmaceutically acceptable salt or stereoisomer thereof in about 0.01%, about 0.02%, about 0.05%, about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, by weight of the composition.

In embodiments of the pharmaceutical composition of the present disclosure, the composition comprises iloprost or a pharmaceutically acceptable salt or stereoisomer thereof at a concentration of about 0.05 mg/mL to about 2 mg/mL, including all values and subranges therebetween. In embodiments, the composition comprises iloprost or a pharmaceutically acceptable salt or stereoisomer thereof at a concentration of about 0.1 mg/mL to about 0.3 mg/mL, including all values and subranges therebetween. In embodiments, the composition comprises iloprost or a pharmaceutically acceptable salt or stereoisomer thereof at a concentration of about 0.2 mg/mL (200 mcg/mL).

In embodiments of the pharmaceutical composition of the present disclosure, the composition comprises iloprost or a pharmaceutically acceptable salt or stereoisomer thereof at a concentration of about 0.5 mg/mL to about 1.5 mg/mL, including all values and subranges therebetween. In embodiments, the composition comprises iloprost or a pharmaceutically acceptable salt or stereoisomer thereof at a concentration of about 0.8 mg/mL to about 1.2 mg/mL, including all values and subranges therebetween. In embodiments, the composition comprises iloprost or a pharmaceutically acceptable salt or stereoisomer thereof at a concentration of about 1 mg/mL.

The present disclosure also provides a sustained release pharmaceutical composition for injection, comprising iloprost or a pharmaceutically acceptable salt or stereoisomer thereof and a pharmaceutically acceptable carrier or excipient; wherein the composition releases iloprost over a period of at least about 48 hours. In embodiments, he composition releases iloprost over a period of at least about 60 hours. In embodiments, he composition releases iloprost over a period of at least about 72 hours.

The present disclosure also provides a sustained release pharmaceutical composition for injection, comprising iloprost or a pharmaceutically acceptable salt or stereoisomer thereof and a pharmaceutically acceptable carrier or excipient; wherein the composition releases iloprost over a period of about 3 days to about 7 days.

The present disclosure also provides a sustained release pharmaceutical composition for injection, comprising iloprost or a pharmaceutically acceptable salt or stereoisomer thereof and a pharmaceutically acceptable carrier or excipient; wherein the composition releases iloprost over a period of about 5 days to about 7 days.

In embodiments of the sustained release pharmaceutical composition provided herein, the composition provides a serum concentration of iloprost at steady state (Css) in the range of about 10 pg/mL to about 100 pg/mL, including all values and subranges therebetween. In embodiments, the composition provides a Css in the range of about 10 pg/mL to about 50 pg/mL, including all values and subranges therebetween. In embodiments, the composition provides a Css in the range of about 10 pg/mL to about 40 pg/mL, including all values and subranges therebetween. In embodiments, the composition provides a Css in the range of about 20 pg/mL to about 40 pg/mL, including all values and subranges therebetween. In embodiments, the composition provides a Css in the range of about 20 pg/mL to about 30 pg/mL, including all values and subranges therebetween. In embodiments, the composition provides a Css of iloprost of about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, about 16 pg/mL, about 17 pg/mL, about 18 pg/mL, about 19 pg/mL, about 20 pg/mL, about 21 pg/mL, about 22 pg/mL, about 23 pg/mL, about 24 pg/mL, about 25 pg/mL, about 26 pg/mL, about 27 pg/mL, about 28 pg/mL, about 29 pg/mL, about 30 pg/mL, about 31 pg/mL, about 32 pg/mL, about 33 pg/mL, about 34 pg/mL, about 35 pg/mL, about 36 pg/mL, about 37 pg/mL, about 38 pg/mL, about 39 pg/mL, about 40 pg/mL, about 41 pg/mL, about 42 pg/mL, about 43 pg/mL, about 44 pg/mL, about 45 pg/mL, about 46 pg/mL, about 47 pg/mL, about 48 pg/mL, about 49 pg/mL, or about 50 pg/mL.

In embodiments of the pharmaceutical composition provided herein, a single dose of the composition comprises about 5.0 mcg iloprost or a pharmaceutically acceptable salt or stereoisomer thereof per weight of the subject (kg) to about 10.0 mcg/kg, including all values and subranges therebetween. In embodiments, the composition comprises about 5.0 mcg, about 5.5 mcg, about 6.0 mcg, about 6.5 mcg, about 7.0 mcg, about 7.5 mcg, about 8.0 mcg, about 8.5 mcg, about 9.0 mcg, about 9.5 mcg, or about 10.0 mcg iloprost or a pharmaceutically acceptable salt or stereoisomer thereof per weight of the subject (kg).

In embodiments of the pharmaceutical composition provided herein, the injection is a subcutaneous injection.

In embodiments of the pharmaceutical composition of the present disclosure, the composition comprises a phospholipid.

In embodiments, phospholipid is lecithin, phosphatidylcholine, phosphotidylethanolamine, phosphotidylserine, phosphatidylinositol, phosphoglyceride, phosphoglycerol, phospholipid, sphingosine, ganglioside, phytosphingosine, diacylglycerol, phosphocholine, phosphoethanolamine, hosphotidylserine, lysophospholipid, pegylated phospholipid, mixed chain phospholipid, or a combination thereof. In embodiments, lecithin is soy lecithin, egg lecithin, or a combination thereof.

In embodiments of the pharmaceutical composition of the present disclosure, the composition comprises a phospholipid selected from lecithin, phosphatidylcholine, or combinations thereof. In embodiments, the phospholipid is lecithin.

In embodiments of the pharmaceutical composition of the present disclosure, the phospholipid is present in about 20% to about 80% by weight of the composition, including all values and subranges therebetween. In embodiments, the phospholipid is present in about 30% to about 60% by weight of the composition. In embodiments, the phospholipid is present in about 30% to about 40% by weight of the composition. In embodiments, the phospholipid is present in about 30%, about 35%, about 40%, about 45%, about 50%, about 55%, or about 60% by weight of the composition.

In embodiments of the pharmaceutical composition of the present disclosure, the particles comprising the phospholipid have an average diameter of about 5 nm to about 500 nm, including values and subranges therebetween. In embodiments, the particles comprising the phospholipid have an average diameter of about 30 nm to about 100 nm, including values and subranges therebetween. In embodiments, the particles comprising the phospholipid have an average diameter of about 5 nm, about 10 nm, about 20 nm, about 30 nm, about 40 nm, about 50 nm, about 60 nm, about 70 nm, about 80 nm, about 90 nm, or about 100 nm.

In embodiments of the pharmaceutical composition of the present disclosure, the composition comprises an oil. In embodiments, oil is synthetic glyceride, natural glyceride, or non-glycerides comprising synthetic triglycerides. In embodiments, oil is synthetic glyceride, natural glyceride, tricaprylin, triolein, or trimyristin, vegetable oil, animal oil, medium chain oil/glycerides, vitamin E, vitamin E acetate, vitamin E succinate, fatty acid, fatty acid monoester, or cholesterol. In embodiments, oil is synthetic oil, a vegetable oil, sesame oil, a medium chain oil, silicone oil, ethyl oleate, fatty acid, vitamin E, vitamin E succinate, cholesterol, triglyceride oil, or a mixture thereof. In embodiments, synthetic oil, a vegetable oil, a medium chain oil, ethyl oleate, fatty acid, vitamin E, vitamin E succinate, cholesterol, or a combination thereof. In embodiments, the oil is liquid at about 35° C. to about 37° C.

In embodiments of the pharmaceutical composition of the present disclosure, the oil is present in about 5% to about 60% by weight of the composition, including all values and subranges therebetween. In embodiments, the oil is present in about 10% to about 50% by weight of the composition. In embodiments, the oil is present in about 10%, about 15%, about 20%, about 25%, about 30%, about 35%, about 40%, about 45%, or about 50% by weight of the composition.

In embodiments of the pharmaceutical composition of the present disclosure, the phospholipid and the oil are present in a ratio of about 4:1 to about 2:1, including all ratios and subranges therebetween. In embodiments of the pharmaceutical composition of the present disclosure, the phospholipid and the oil are present in a ratio of about 2:1 to about 1:1, including all ratios and subranges therebetween. In embodiments of the pharmaceutical composition of the present disclosure, the phospholipid and the oil are present in a ratio of about 1.5:1 to about 1:1, including all ratios and subranges therebetween.

In embodiments of the pharmaceutical composition of the present disclosure, the pharmaceutically acceptable excipient is sucrose, dextrose, lactose, glucose, trehalose, maltose, mannitol, sorbitol, glycerol, amylose, starch, amylopectin, triglycerides, fatty acids, carbohydrates, or a combination thereof. In embodiments of the pharmaceutical composition of the present disclosure, the pharmaceutically acceptable excipient is sucrose, dextrose, lactose, glucose, trehalose, maltose, mannitol, sorbitol, glycerol, amylose, starch, amylopectin, or a combination thereof.

In embodiments of any one of the pharmaceutical composition of the present disclosure, the pharmaceutically acceptable excipient is an acidifying agent, an alkalizing agent, a pH buffering agent, a metal ion chelator, an antioxidant, a preservative, a tonicity/osmotic pressure modifier, a condensing agent, a solubilizing agent, or a combinations thereof.

In embodiments, an acidifying agent is hydrochloric acid, acetic acid, sulfuric acid, or the like. In embodiments, an acidifying agent is used to adjust the pH of the composition.

In embodiments, an alkalizing agent is sodium hydroxide, potassium hydroxide, ammonium hydroxide, lysine, arginine, or the like. In embodiments, an alkalizing agent is used to adjust the pH of the composition. In embodiment, the alkalizing agent is sodium hydroxide.

In embodiments, a pH buffering agent is phosphate buffer, acetate buffer, citrate buffer, phosphate citrate buffer, bicarbonate buffer, tartrate buffer, trometamol buffer, histidine buffer, TRIS buffer, or the like.

In embodiments, a metal ion chelator is disodium edetate (EDTA), glycine, citric acid or a pharmaceutically acceptable salt thereof.

In embodiments, an antioxidant is ascorbic acid, ascorbyl palmitate, sodium metabisulfite, propyl gallate, butylated hydroxyanisole, butylated hydroxytoluene, tocopherol, methionine, citric acid, reducing sugars, or a pharmaceutically acceptable salts thereof, or combinations thereof.

In embodiments a preservative inhibits bacteria or fungi growth in the composition. In embodiments, a preservative is cresols, phenol, benzyl alcohol, ethanol, chlorobutanol, parabens, imidura, or benzylkonium chloride.

In embodiments, a tonicity/osmotic pressure modifier adjusts the osmolality of the composition. In embodiments, a tonicity/osmotic pressure modifier is potassium chloride, sodium chloride, trehalose, sucrose, sorbitol, glycerol, mannitol, polyethylene glycol, propylene glycol, albumin, amino acid, or combinations thereof. In embodiments, tonicity agents include, but are not limited to, glycerin, lactose, mannitol, dextrose, sodium chloride, sodium sulfate, and sorbitol. In embodiments, the tonicity agent is sodium chloride. In embodiments, the tonicity agent is about 0.2% to about 0.9% sodium chloride. In embodiments of the pharmaceutical composition of the present disclosure, the composition has an osmolality of about 300 mOsm.

In embodiments, a condensing agent reduces solubility, alters release rate or increases stability of iloprost or a pharmaceutically acceptable salt or stereoisomer thereof or a stereoisomer thereof. In embodiments, a condensing agent is glutaraldehyde, acetaldehyde, zinc salts, aluminum salts, iron salts, protamine, or the like.

In embodiments, a solubilizing agent is cyclodextrin or a surfactant. In embodiments, surfactant is polysorbates, bile salt, lecithin, sodium dodecyl sulfate, poloxamer, polyethoxylated castor oil, polyoxylglycerides (e.g., polyethylene glycol fatty acid esters), or sorbitan stearates. In embodiments, polysorbate is polysorbate 80. In embodiments, solubilizing agents is selected from pharmaceutically acceptable alcohols, glycols, esters, ethers, or silicones. In embodiments, the solubilizing agent is ethanol.

In embodiments of the pharmaceutical composition of the present disclosure, the pharmaceutically acceptable carrier is solvent. In embodiments, the pharmaceutically acceptable carrier is solvent is water, ethanol, propylene glycol, glycerol, sorbitol, polyethylene glycol, silicone oil, glycofurol, ethyl oleate, or a combination thereof. In embodiments of the pharmaceutical composition of the present disclosure, the pharmaceutically acceptable carrier or solvent is ethanol, propylene glycol, glycerol, sorbitol, polyethylene glycol, ethyl oleate, or a mixture thereof.

In embodiments, solvents are sterile solvents. In embodiments, solvents include, but are not limited to, water, glucose solution, dextrose solution, saline solution, Ringer's solution, and lactated Ringer's solution. In embodiments, the solvent is water.

In embodiments of the pharmaceutical composition of the present disclosure, the pharmaceutically acceptable carrier or solvent is water. In embodiments, the composition comprises water in about 0.05% to about 75% by weight of the composition, including all values and subranges therebetween. In embodiments, the composition comprises water in about 0.1% to about 65% by weight of the composition, including all values and subranges therebetween. In embodiments, the composition comprises water in about 10% to about 70% by weight of the composition, including all values and subranges therebetween. In embodiments, the composition comprises water in about 40% to about 60% by weight of the composition, including all values and subranges therebetween.

In embodiments of the pharmaceutical composition of the present disclosure, the composition is an injectable gel. In embodiments, the composition is a single-phase gel. In embodiments, the composition is a phospholipid gel.

In embodiments, the pharmaceutical composition comprises any phospholipids or compositions thereof as described in U.S. Pat. No. 9,517,202, which is hereby incorporated by reference in its entirety for all purposes.

In embodiments of any one of the pharmaceutical compositions provided herein, the composition comprises a liposome. In embodiments, the liposome comprises phosphatidyl choline, phosphatidyl glycerol, phosphatidyl serine, phosphatidic acid, cholesterol, cholesterol derivatives, plant sterol, phosphatidyl ethanolamine, distearoylphophatidyl ethanolamine, pegylated phosphatidyl ethanolamine, pegylated distearoylphosphatidyl ethanolamine, neutral phospholipid, neutral lipid, cationic lipids, amphipathic lipid, charged phospholipid, or combinations thereof. In embodiments, the liposome comprises PEG-distearoylphosphatidyl ethanolamine, dimyristoylphosphatidyl choline (DMPC), distearoylphosphatidyl choline (DSPC), dipalmitoyl phosphatidyl choline (DPPC), dimyristoylphosphatidyl glycerol (DMPG), dipalmitoylphosphatidyl glycerol (DPPG), distearoylphosphatidyl glycerol (DSPG), dimyristoylphosphatidyl serine (DMPS), dipalmitoylphosphatidyl serine (DPPS), distearoylphosphatidyl serine (DSPS), dimyristoylphosphatidic acid (DMPA), dipalmitoylphosphatidic acid (DPPA), cholesterol hemisuccinate (CHEMS), hydrogenated soy bean phosphatidyl choline (HSPC), distearoyl phosphatidyl choline (DSPC), hydrogenated egg phosphatidyl choline (HEPC), soy bean phosphatidyl choline (SPC), egg phosphatidyl choline (EPC), dilauroyl phosphatidylglycerol, dipalmitoyl phosphatidylglycerol, distearoyl phosphatidyl glycerol, dimyristate phosphatidylglycerol, dioleic acid phosphatidylserine, dioleoyl phosphatidylglycerol, dilauroyl phosphatidic acid, dimyristate phosphatidic acid, distearoyl phosphatidic acid, or combinations thereof. In embodiments, the liposome comprises a lipid, phospholipid, cholesterol, or a combination thereof.

In embodiments, the cationic lipid is cholesteryl-3β-N-(dimethylaminoethyl) carbamate (DC-Chol), 3-β-[N—(N,N′-dimethylaminoethane)carbamoyl]cholesterol (DAC-Chol), (N-[1-(2,3-dimyristoyloxy)propyl]-N,N,N-trimethylammonium salt (DMTAP), (N-(1-(2,3-dipalmitoyloxy)propyl]-N,N,N-trimethylammonium salt (DPTAP), (N-[1-(2,3-dioleoyloxy)propyl]-N,N,N-trimethylammonium salt (DOTAP), 3-β-[N—(N′,N′,N′-trimethylaminoethane)carbamo-yl]cholesterol (TC-Chol), bis-guanidinium-spermidine-cholesterol (BGSC), bis-guanidinium-tren-cholesterol (BGTC), (1,3-dioleoyloxy-2-(6-carboxyspermyl)propylamide) (DOSPER), (1,2-dioleyloxypropyl)-N,N,N-trimethylammonium chloride (DOTMA) (Lipofectin R), (1,2-dioleyloxypropyl)-3-dimethylhydroxyethyl-ammonium bromide (DORIE), 1,2-dioleoyl-3-succinyl-sn-glycero choline ester (DOSC), 1,2-dioleoyl-sn-glycero-3-succinyl-2-hydroxy-ethyl disulfide ornithine (DOGSDSO), dimethyldioctadecylammonium bromide (DDAB), ((C18)2GlySper3+) N,N-dioctadecylamido-glycyl-spermine (DOGS) (Transfectam®), (C18)2Gly+N,N-dioctadecylamidoglycine, 1,2-dioleoyl-sn-glycero-3-ethylphosphocholine (DOEPC), O-alkylphosphatidyl cholines, ethanolamines, 1,3-bis(1,2-bis-tetradecyloxy-propyl-3-dimethylethoxyammonium bromide)-propan-2-ol (Neophectin®), saturated derivatives with dimyristoyl, dipalmitoyl or distearoyl chains thereof, unsaturated fatty acid derivatives thereof, derivatives with fatty alcohol chains thereof, or combinations thereof.

In embodiments, the neutral lipid is triglyceride, propylene glycol ester, ethylene glycol ester, squalene, or the like. In embodiments, the triglyceride is triolein, tripalmitolein, trimyristolein, trilinoelin, tributyrin, tricaproin, tricaprylin, or tricaprin. In embodiments, the triglyceride can be a mixed chain triglyceride (e.g., one or more of the fatty acid chains in the triglyceride is different from another).

In embodiments, the amphipathic lipid is a lipid having a hydrophobic region and a hydrophilic region in the same molecule. An amphipathic lipid can be cationic or anionic. In embodiments, the amphipathic lipid is a zwitterionic phospholipid. In embodiments, the amphipathic lipid is phosphatidyclholine, phosphatidylethanolamine, sphingomyelin, lysophosphatidylcholine, lysophosphatidylethanolamine, phosphatidylglycerol, phosphatidylserine, phosphatidylinositol, phosphatidic acid, cardiolipin, acyl trimethylammonium propane, diacyl dimethylammonium propane, stearylamine, dioleyl phosphatidyl choline (DOPC), dierucoylphosphatidylcholine (DEPC), or dipalmitoylphosphatidylglycerol (DPPG).

In embodiments, the phospholipid is aglycerophosphoric acid such as lecithin; phosphatidylglycerol; phosphatidic acid; phosphatidylethanolamine; phosphatidylserine; phosphatidylinositol; sphingophospholipids such as sphingomyelin; natural or synthetic diphosphatidylphospholipids such as cardiolipin; hydrogenated phospholipids such as hydrogenated soybean phosphatidylcholine (HSPC), hydrogenated yolk phosphatidylcholine, distearoylphosphatidylcholine, dipalmitoylphosphatidylcholine and dimyristoylphosphatidylcholine; or the like; or derivatives thereof.

In embodiments of any one of the pharmaceutical compositions provided herein, the liposome composition comprises a stabilizer. In embodiments, the stabilizer is ethylene diamine tetraacetic acid or a pharmaceutically acceptable salt thereof.

In embodiments, the liposome composition comprises an antioxidant. In embodiments, the antioxidant is vitamin E, ascorbic acid, uric acid, or a derivative thereof. In embodiments, the liposome composition comprises a reagent that enhances the binding affinity of the liposome (such as palmitic acid).

In embodiments, the liposome is multivesicular liposome (MVL).

In embodiments of any one of the pharmaceutical compositions provided herein, the iloprost or a pharmaceutically acceptable salt or stereoisomer thereof is entrapped or encapsulated in the liposome.

In embodiments of any one of the pharmaceutical compositions provided herein, the liposome has a mean particle diameter in the range of about 10 nm to about 1,000 nm, including all values and subranges therebetween. In embodiments, the liposome has a mean particle diameter in the range of about 10 nm to about 300 nm. In embodiments, the liposome has a mean particle diameter in the range of about 10 nm to about 150 nm.

In embodiments of any one of the pharmaceutical compositions provided herein, the liposome can be prepared by conventional methods, such as gradient method or remote loading method.

In embodiments of any one of the pharmaceutical compositions provided herein, the composition comprises microspheres. In embodiments, the microspheres are polymer microspheres.

In embodiments of any one of the pharmaceutical composition provided herein, the microspheres comprise polylactide polymer, sodium acrylate polymer, acrylamide polymer, acrylamide derivative polymer or copolymer, sodium acrylate and vinyl alcohol copolymer, a vinyl acetate and acrylic acid ester copolymer, a vinyl acetate and methyl maleate copolymer, and a isobutylene-maleic anhydride crosslinked copolymer, polylactic acid (PLA), polyglycolic acid (PGA), a copolymer of PLA and PGA, poly-L-lactide (PLLA), poly-D,L-lactide (PDLA), poly-capralactone (PCL), poly(D,L-lactide-co-glycolide), poly(3-hydroxy-butyrate), polylactone, polyanhydride, poly(hydroxy-butyrate)-co-(hydroxy-valerate), polypropylene-glucose, poly(lactic acid)-polyglycol, and poly (hydroxyacetic acid)-polyglycol, or a combination thereof.

In embodiments of any one of the pharmaceutical compositions provided herein, the microspheres comprise a biodegradable wall.

In embodiments of any one of the pharmaceutical compositions provided herein, the microspheres have a mean diameter of about 1 μm to about 500 μm, including all values and subranges therebetween. In embodiments, the microspheres have a mean diameter of about 10 μm to about 300 μm. In embodiments, the microspheres have a mean diameter of about 10 μm to about 150 μm.

In embodiments of any one of the pharmaceutical compositions provided herein, the iloprost or a pharmaceutically acceptable salt or stereoisomer thereof is entrapped or encapsulated in the microsphere.

In embodiments of any one of the pharmaceutical compositions provided herein, the composition comprises a gel. In embodiments, the gel is a phospholipid gel. In embodiments, the gel is a polymer gel. In embodiments, the gel is an aqueous gel. In embodiments, the gel is an anhydrous gel.

In embodiments of any one of the pharmaceutical composition provided herein, the gel comprises xanthan gum, deacylated xanthan gum, a carboxymethyl ether, propylene glycol ester, homo polysaccharide gum, galactomannan gum, hydroxypropyl cellulose, hydroxypropylmethyl cellulose, polylactide polymer, sodium acrylate polymer, acrylamide polymer, acrylamide derivative polymer or copolymer, sodium acrylate and vinyl alcohol copolymer, a vinyl acetate and acrylic acid ester copolymer, a vinyl acetate and methyl maleate copolymer, and a isobutylene-maleic anhydride crosslinked copolymer, polylactic acid (PLA), polyglycolic acid (PGA), a copolymer of PLA and PGA, poly-L-lactide (PLLA), poly-D,L-lactide (PDLA), poly-capralactone (PCL), poly(D,L-lactide-co-glycolide), poly(3-hydroxy-butyrate), polylactone, polyanhydride, poly(hydroxy-butyrate)-co-(hydroxy-valerate), polypropylene-glucose, poly(lactic acid)-polyglycol, and poly(hydroxyacetic acid)-polyglycol, or a combination thereof. In embodiments, gel comprises phospholipids as disclosed herein.

In embodiments of any one of the pharmaceutical compositions provided herein, the iloprost or a pharmaceutically acceptable salt or stereoisomer thereof is entrapped or encapsulated in the gel.

In embodiments of the pharmaceutical composition of the present disclosure, the composition is nanodispersion, nanoemulsion or nanosuspension. In embodiments of the pharmaceutical composition of the present disclosure, the composition is nanoemulsion. In embodiments, the composition is transparent, translucent, or opaque. In embodiments, the composition is semi-solid. In embodiments, nanodispersion, nanoemulsion or nanosuspension results from mixing the phospholipid and water. In embodiments, nanodispersion, nanoemulsion or nanosuspension results from mixing phospholipids and water, and homogenizing the mixture. In embodiments, nanodispersion, nanoemulsion or nanosuspension results from mixing the phospholipid, oil, and water. In embodiments, nanodispersion, nanoemulsion or nanosuspension results from mixing the phospholipid, oil, and water, and homogenizing the mixture.

In embodiments of any one of the pharmaceutical compositions provided herein, the composition comprises a peptide.

In embodiments, the peptide comprises a combination of acidic, neutral, and/or basic amino acids. In embodiments, the peptide comprises a primarily acidic peptide fragment and a primarily basic peptide fragment, with a neutral peptide fragment in between the acidic and basic fragments. In embodiments, the peptide comprises two or more primarily acidic peptide fragments, wherein a neutral peptide fragment exists between the different primarily acidic peptide fragments. In embodiments, the peptide comprises two or more primarily basic peptide fragments, wherein a neutral peptide fragment exists between the different primarily basic peptide fragments.

In embodiments, a primarily acidic peptide fragment comprises at least 75% acidic amino acids. In embodiments, the primarily acidic peptide fragment comprises at least 80%, at least 85%, at least 90%, or at least 95% acidic amino acids. In embodiments, the primarily acidic peptide fragment comprises 100% acidic amino acids. In embodiments, the primarily acidic peptide fragment comprises all acidic amino acids except for one or two neutral amino acids.

In embodiments, a primarily basic peptide fragment comprises at least 75% basic amino acids. In embodiments, the primarily basic peptide fragment comprises at least 80%, at least 85%, at least 90%, or at least 95% basic amino acids. In embodiments, the primarily basic peptide fragment comprises 100% basic amino acids. In embodiments, the primarily basic peptide fragment comprises all basic amino acids except for one or two neutral amino acids.

In embodiments, a neutral peptide fragment comprises all neutral amino acids except for one or two acidic or basic amino acids. In embodiments, a neutral peptide fragment comprises all neutral amino acids.

In embodiments, the peptide comprises about 5 to about 30 amino acids, including all values and subranges therebetween. In embodiments, the peptide comprises about 9 to about 25 amino acids, including all values and subranges therebetween. In embodiments, the peptide comprises about 9 to about 21 amino acids, including all values and subranges therebetween.

In embodiments, the peptide is RRRRRRLLLAAAEEE (SEQ ID NO. 1), RRRRRRLLLAAAEEE (SEQ ID NO. 2), KKKKKKLLLAAAEEE (SEQ ID NO. 3), RRRRRLLLAAAEE (SEQ ID NO. 4), RRRLLLEEE (SEQ ID NO. 5), RRRRRLLLEEEEE (SEQ ID NO. 6), RRRRRLLLLLEEEEE (SEQ ID NO. 7), RRRRRRRLLLLLLLEEEEEEE (SEQ ID NO. 8), EEEEELLLLLRRRRR (SEQ ID NO. 9), or RRRRRLLLLLDDDDD (SEQ ID NO. 10).

In embodiments of the composition comprising a peptide, the composition further comprises a solvent. In embodiments, the solvent is water, tetrahydrofuran, dimethylformamide, dimethylsulfoxide or acetonitrile, or a combination thereof.

In embodiments of the composition comprising a peptide, the composition further comprises a tonicity agent. In embodiments, the tonicity agent is as described herein. In embodiments, the tonicity agent is about 0.2% to about 0.9% sodium chloride.

In embodiments, the peptide allows for the controlled or sustained release of iloprost or a pharmaceutically acceptable salt or stereoisomer thereof. In embodiments, the release can be controlled by varying the weight ratio of iloprost or a pharmaceutically acceptable salt or stereoisomer thereof to the peptide. In embodiments, the weight ratio of iloprost or a pharmaceutically acceptable salt or stereoisomer thereof to the peptide is about 1:0.1 to about 1:100, including all ratios and subranges therebetween. In embodiments, the weight ratio of iloprost or a pharmaceutically acceptable salt or stereoisomer thereof to the peptide is about 1:1 to about 1:80, about 1:2 to about 1:20, about 1:2 to about 1:10, or about 1:0.5 to about 1:12, including all ratios and subranges therebetween. In embodiments, the weight ratio of iloprost or a pharmaceutically acceptable salt or stereoisomer thereof to the peptide is about 1:3.

In embodiments, the mole ratio of iloprost or a pharmaceutically acceptable salt or stereoisomer thereof to the peptide is about 1:0.1 to about 1:30, including all ratios and subranges therebetween. In embodiments, the mole ratio of iloprost or a pharmaceutically acceptable salt or stereoisomer thereof to the peptide is about 1:0.1 to about 1:20, about 1:0.5 to about 1:8, or about 1:0.75 to about 1:7, including all ratios and subranges therebetween.

In embodiments, the peptide is non-covalently bonded with iloprost or a pharmaceutically acceptable salt or stereoisomer thereof. In embodiments, the peptide encapsulates or associates with iloprost or a pharmaceutically acceptable salt or stereoisomer thereof. In embodiments, the peptide self-organizes around iloprost or a pharmaceutically acceptable salt or stereoisomer thereof. In embodiments, the self-organization is by intramolecular and/or intermolecular stacking. In embodiments, the peptide and iloprost or a pharmaceutically acceptable salt or stereoisomer thereof are stacked through non-covalent bonding.

In embodiments, the peptide can be any one of the peptides as described in U.S. Patent Publication No. 2019/0307834, which is hereby incorporated by reference in its entirety for all purposes.

In embodiments of the pharmaceutical composition of the present disclosure, the composition is a depot composition.

In embodiments of any one of the pharmaceutical compositions as disclosed herein, the composition has a pH in the range of about 8.0 to about 9.0, include all values and subranges therebetween. In embodiments, the composition has a pH in the range of about 8.0 to about 8.9. In embodiments, the composition has a pH of about 8.3. In embodiments, the pH of the composition is adjusted with an alkalizing agent and/or an acidifying agent. In embodiments, the pH of the composition is adjusted with sodium hydroxide.

In embodiments of any one of the pharmaceutical compositions as disclosed herein, the composition comprises an alkalizing agent as described herein. In embodiments of any one of the pharmaceutical compositions as disclosed herein, the composition comprises a pH buffering agent as disclosed herein.

In embodiments of any one of the pharmaceutical compositions as disclosed herein, the composition is prepared under inert atmosphere or under anaerobic conditions. In embodiments, the composition is prepared using inert or oxygen-free techniques. In embodiments, the composition is prepared using degassed or nitrogen-sparged materials. In embodiments, the composition is sparged with an inert gas, such as nitrogen, or blanketed with an inert gas before packaging, capping, or sealing the container which the composition is prepared or contained in.

In embodiments of the pharmaceutical composition of the present disclosure, the composition is injectable through a 21 G needle, 22 G needle, 23 G needle, 24 G needle, 25 G needle, 26 G needle, 27 G needle, or 28 G needle. In embodiment, the composition is injectable through a 25 G needle. In embodiments, the needle is about ½-inch, about 5/16-inch, or about 3/16-inch long. In embodiments, the needle is about ½-inch long.

In embodiments of the pharmaceutical composition of the present disclosure, the composition is injectable with an about 0.05 cc, about 0.1 cc, about 0.2 cc, about 0.3 cc, about 0.5 cc, about 1.0 cc, or about 1.5 cc syringe. In embodiments, the composition is injectable with a 1 cc syringe.

In embodiments of the pharmaceutical composition of the present disclosure, the composition is injectable at an extrusion rate of about 0.5 cc/min to about 4 cc/min, including all values and subranges therebetween. In embodiments, the composition is injectable at an extrusion rate of about 1 cc/min to about 2 cc/min. In embodiments, the composition is injectable at an extrusion rate of about 2 cc/min.

In embodiments of the pharmaceutical composition of the present disclosure, the force applied at injection is in the range of about 5 pounds to about 25 pounds, including all values and subranges therebetween. In embodiments, the force applied at injection is about 10 pounds to about 15 pounds, including all values and subranges therebetween. In embodiments, the force applied at injection 12 pounds or less.

In embodiments of the pharmaceutical composition of the present disclosure, the composition is injectable through a 25 G, ½-inch long needle from a 1 cc syringe at an extrusion rate of about 2 cc/min by an applied force of 12 pounds or less.

In embodiments of the pharmaceutical composition of the present disclosure, the composition can be self-administered by the patient.

In embodiments of the pharmaceutical composition of the present disclosure, the composition is stable at room temperature (20° C. to 25° C.) for at least about 1 month, 2 months, or 3 months, including all values therebetween. In embodiments, the composition is stable at room temperature (20° C. to 25° C.) for at least 6 months or at least 12 months, including all values therebetween.

In embodiments of the pharmaceutical composition of the present disclosure, the composition is stable at refrigerated temperatures (2° C. to 8° C.) for at least about 1 month, 2 months, or 3 months, including all values therebetween. In embodiments, the composition is stable at refrigerated temperatures (2° C. to 8° C.) for at least 6 months or at least 12 months.

In embodiments of the pharmaceutical composition of the present disclosure, the composition is superior to the iloprost infusion treatment (over 6 hours a day for 5 consecutive days) because the subject does not undergo peripheral or peripherally inserted central catheter (PICC) placement or receive the lengthy continuous infusion treatments for consecutive days. Thus, the injection composition of the disclosure reduces the patients risk to nosocomial infections (contracting infection at a hospital or infusion center), catheter related safety complications (infections, infiltration, extravasation, site pain), lessens the economic impact to the health care system (self-administration/self-injection) and improves health outcomes due to superior treatment adherence.

### Therapeutic Use

The present disclosure relates to methods for treating symptomatic digital ischemic episodes (symptomatic RP) or digital ulcers in a systemic sclerosis (SSc) patient. Currently, there are no FDA-approved therapies available to improve symptoms in patients with SSc. Also, there are no formal guidelines available for evaluation and treatment of symptomatic digital ischemic episodes in a SSc patient. Available standard of care for SSc patients with symptomatic RP has several limitations and is mainly focused on nonpharmacological therapy, such as avoidance of cold temperatures, keeping whole body warm, and avoid smoking. Emotional stress alone can trigger worsening symptoms of RP. In addition, patients are instructed to avoid pharmacotherapy agents that can cause vasoconstriction (e.g. sympathomimetic drugs, clonidine, serotonin-receptor agonists). Oral vasodilator therapies are used; however, the evidence of efficacy and safety is lacking. Proximal sympathectomy can improve symptoms in some patients but the effects last less than one year and the procedure is costly and invasive. Thus, there is a need for an effective treatment of symptomatic RP in SSc patients.

The present disclosure relates to method for treatment of systemic sclerosis (SSc) to reduce the frequency of symptomatic digital ischemic episodes (symptomatic RP), comprising administering any one of the composition comprising iloprost or a pharmaceutically acceptable salt or stereoisomer thereof, as disclosed herein, to a subject in need thereof.

The present disclosure also relates to method for treatment of SSc to reduce the severity of digital ischemic episodes (RP), comprising administering any one of the compositions comprising iloprost or a pharmaceutically acceptable salt or stereoisomer thereof, as disclosed herein, to a subject in need thereof.

The present disclosure also relates to method for treatment of SSc to reduce the frequency and severity of symptomatic digital ischemic episodes (symptomatic RP), comprising administering any one of the composition comprising iloprost or a pharmaceutically acceptable salt or stereoisomer thereof, as disclosed herein, to a subject in need thereof.

In embodiments, an advantage of subcutaneous or intramuscular injection of iloprost or a pharmaceutically acceptable salt thereof or a stereoisomer thereof, is its greater bioavailability when compared to other routes of administration. For example, iloprost (a synthetic analog of prostacyclin, PGI2) has poor oral bioavailability and tolerability making oral administration route not viable.

In embodiments, iloprost or a pharmaceutically acceptable salt thereof or a stereoisomer thereof, increases cyclic AMP concentrations in pertinent cells thereby having an effect as vasodilator or as an anti-vasoconstrictor, anti-fibrotic, anti-platelet, and/or anti-inflammatory. In embodiments, once iloprost or a pharmaceutically acceptable salt thereof or a stereoisomer thereof, reaches the site of action (digit cutaneous circulation), the injection iloprost therapy would reduce the frequency, symptoms and duration of symptomatic RP attacks in SSc patients by attenuating digital vasoconstrictive episodes, fibrosis, inflammation, and platelet activation.

In embodiments, the composition comprising iloprost or a pharmaceutically acceptable salt or stereoisomer thereof, is administered once a day. In embodiments, the composition is administered twice a week. In embodiments, the composition is administered once a week.

In embodiments, one cycle of treatment with once-a-day composition comprising iloprost or a pharmaceutically acceptable salt or stereoisomer thereof, is about 3 days to about 10 days administered daily. In embodiments, one cycle of treatment with once-a-day composition comprising iloprost or a pharmaceutically acceptable salt or stereoisomer thereof, is about 4 days or about 5 days.

In embodiments, one cycle of treatment with once-a-day composition comprising iloprost or a pharmaceutically acceptable salt or stereoisomer thereof, includes one or more days of continuous infusion of iloprost or a pharmaceutically acceptable salt or stereoisomer thereof over 6 hours/day at 0.5 to 2.0 ng iloprost/kg body weight/min. In embodiments, one cycle of treatment with once-a-day composition comprising iloprost or a pharmaceutically acceptable salt or stereoisomer thereof, includes one day of continuous infusion of iloprost or a pharmaceutically acceptable salt or stereoisomer thereof over 6 hours/day at 0.5 to 2.0 ng iloprost/kg body weight/min. In embodiments, one day of continuous infusion of iloprost or a pharmaceutically acceptable salt or stereoisomer thereof over 6 hours/day at 0.5 to 2.0 ng iloprost/kg body weight/min is useful to determine the subject's tolerated dose. In embodiments, on the first day of continuous infusion of iloprost or a pharmaceutically acceptable salt or stereoisomer thereof, the infusion is initiated at a starting dose of 0.5 ng/kg/min with dose increase occurring every 30 minutes (±5 minutes) in increments of 0.5 ng/kg/min up to 2.0 ng/kg/min or the individual tolerated dose. If dose-limiting adverse events (e.g., headache, flushing, jaw pain, myalgia, nausea, or vomiting) occur that cannot be tolerated by the subject, then the dose is to be reduced in a stepwise manner by 0.5 ng/kg/min every 30 minutes (±5 minutes), until a tolerated dose is determined. In embodiments, the subject's tolerated dose determined by the infusion determines the dose of the once-a-day injection composition to be used.

In embodiments, one cycle of treatment with once-a-day composition comprising iloprost or a pharmaceutically acceptable salt or stereoisomer thereof, includes one day of continuous infusion of iloprost or a pharmaceutically acceptable salt or stereoisomer thereof over 6 hours/day at 0.5 to 2.0 ng iloprost/kg body weight/min followed by 3 to 10 dose of once-a-day composition comprising iloprost or a pharmaceutically acceptable salt or stereoisomer thereof. In embodiments, one cycle of treatment with once-a-day composition comprising iloprost or a pharmaceutically acceptable salt or stereoisomer thereof, includes one day of continuous infusion of iloprost or a pharmaceutically acceptable salt or stereoisomer thereof over 6 hours/day at 0.5 to 2.0 ng iloprost/kg body weight/min followed by 4 to 10 days of administering once-a-day composition comprising iloprost or a pharmaceutically acceptable salt or stereoisomer thereof. In embodiments, one cycle of treatment with once-a-day composition comprising iloprost or a pharmaceutically acceptable salt or stereoisomer thereof, includes one day of continuous infusion of iloprost or a pharmaceutically acceptable salt or stereoisomer thereof over 6 hours/day at 0.5 to 2.0 ng iloprost/kg body weight/min followed by 4 to 5 days of administering once-a-day composition comprising iloprost or a pharmaceutically acceptable salt or stereoisomer thereof. In embodiments, where the subject has previously received a treatment of continuous infusion of iloprost or a pharmaceutically acceptable salt or stereoisomer thereof over 6 hours/day at 0.5 to 2.0 ng iloprost/kg body weight/min, a treatment with once-a-day composition can be initiated based on previously determined tolerated dose—i.e., the subject does not need a one or more days of continuous infusion of iloprost or a pharmaceutically acceptable salt or stereoisomer thereof over 6 hours/day at 0.5 to 2.0 ng iloprost/kg body weight/min in the subject's treatment cycle. In embodiments, where the subject has previously received a treatment with iloprost or a pharmaceutically acceptable salt or stereoisomer thereof and the subject's tolerated dose is established, a treatment with once-a-day composition can be initiated based on previously determined tolerated dose.

In embodiments, one cycle of treatment with once-a-day composition comprising iloprost or a pharmaceutically acceptable salt or stereoisomer thereof, includes increasing the dose for the first 2 to 4 day followed by 3 to 5 days at a subject's tolerated dose. In embodiments, the subject receives the lowest dose (Dose 1 of 4 increasing doses) of once-a-day composition on Day 1. In embodiments, the subject receives the second lowest dose (Dose 2) of once-a-day composition on Day 2. If the subject tolerated Dose 2 on Day 2, then, in embodiments, the subject receives the third lowest dose (Dose 3) on Day 3. If the subject did not tolerate Dose 2 on Day 2, then, in embodiments, the subject receives Dose 1 for the remainder of the treatment cycle-Day 3 to Day 5, Day 3 to Day 6, or Day 3 to Day 7. If the subject tolerated Dose 3 on Day 3, then, in embodiments, the subject receives the highest dose (Dose 4) on Day 4. If the subject did not tolerate Dose 3 on Day 3, then, in embodiments, the subject receives Dose 2 for the remainder of the treatment cycle-Day 4 to Day 5, Day 4 to Day 6, or Day 4 to Day 7. If the subject tolerated Dose 4 on Day 4, then, in embodiments, the subject receives Dose 4 for the remainder of the treatment cycle-Day 5, Day 5 to Day 6, or Day 5 to Day 7. If the subject did not tolerate Dose 4 on Day 4, then, in embodiments, the subject receives Dose 3 for the remainder of the treatment cycle-Day 5, Day 5 to Day 6, or Day 5 to Day 7. In embodiment, Dose 1 is about 180 ng iloprost/kg of subject' body weight. In embodiment, Dose 2 is about 360 ng iloprost/kg of subject' body weight. In embodiment, Dose 3 is about 540 ng iloprost/kg of subject' body weight. In embodiment, Dose 4 is about 720 ng iloprost/kg of subject' body weight.

In embodiments, one cycle of treatment with once-a-week composition comprising iloprost or a pharmaceutically acceptable salt or stereoisomer thereof, is about 1 week to about 5 weeks, administered once a week.

In embodiments, the dose of iloprost or a pharmaceutically acceptable salt or stereoisomer thereof, is reduced in subjects with impaired liver function. In embodiments, the dose of iloprost or a pharmaceutically acceptable salt or stereoisomer thereof, is reduced in subjects with Child-Pugh Class B or Class C hepatic impairment.

In embodiments, iloprost or a pharmaceutically acceptable salt thereof or a stereoisomer thereof is administered to a SSc subject with symptomatic RP who experiences symptomatic RP attacks, on average, at least 3 days/week. In embodiments, one episode of symptomatic RP attack comprises (i) at least one color change of the subject's fingers (blue, white, or red) and (ii) at least one symptom of the fingers selected from pain, numbness, tingling, or discomfort. In one embodiment, an episode of symptomatic RP attack is over when the color of the subject's fingers return to the color pre-RP attack and the symptoms return to the subject's pre-RP attack level.

In embodiments, the subject keeps a RP diary to record the frequency, duration, intensity or severity of each symptom, and/or the symptomatic RP attack's impact on the quality of subject's life. In one embodiment, the subject keeps a RP diary before and after the iloprost treatment. In embodiments, the RP diary (a patient-reported outcome; PRO) kept by the patient is useful in assessing the effectiveness of iloprost treatment. In embodiments, the RP diary is maintained electronically (ePRO).

In embodiments, the subject records the number (frequency) of symptomatic RP attacks in a day in the RP diary.

In embodiments, the subject records the duration of each symptomatic RP attack in a day in the RP diary.

In embodiments, the subject records the intensity (severity) of the pain in the fingers associated with symptomatic RP attacks in a given day as a numeric score in the RP diary. In embodiments, the score associated with pain recorded in the RP diary is the score of the worst pain the subject experienced in a given day. In embodiments, the numeric rating score is on a scale of 0 to 10, where 0 is no pain and 10 is severe pain.

In embodiments, the subject records the intensity (severity) of the numbness in the fingers associated with symptomatic RP attacks in a given day as a numeric score in the RP diary. In embodiments, the score associated with numbness recorded in the RP diary is the score of the worst numbness the subject experienced in a given day. In embodiments, the numeric rating score is on a scale of 0 to 10, where 0 is no numbness and 10 is severe numbness.

In embodiments, the subject records the intensity (severity) of the tingling in the fingers associated with symptomatic RP attacks in a given day as a numeric score in the RP diary. In embodiments, the score associated with tingling recorded in the RP diary is the score of the worst tingling the subject experienced in a given day. In embodiments, the numeric rating score is on a scale of 0 to 10, where 0 is no tingling and 10 is severe tingling.

In embodiments, the subject records the intensity (severity) of the discomfort in the fingers associated with symptomatic RP attacks in a given day as a numeric score in the RP diary. In embodiments, the score associated with discomfort recorded in the RP diary is the score of the worst discomfort the subject experienced in a given day. In embodiments, the numeric rating score is on a scale of 0 to 10, where 0 is no discomfort and 10 is severe discomfort.

The present disclosure also relates to reducing the weekly average frequency of symptomatic RP attacks from baseline in a SSc subject experiencing symptomatic attacks, comprising administering iloprost or a pharmaceutically acceptable salt thereof or a stereoisomer thereof. In embodiments, iloprost or a pharmaceutically acceptable salt thereof or a stereoisomer thereof is administered in any method as disclosed herein.

In embodiments, the weekly average frequency of symptomatic RP attacks is determined according to the subject's RP diary. The baseline weekly average frequency of symptomatic RP attacks is the weekly average of the number of symptomatic RP attack episodes in the subject before iloprost treatment. In embodiments, the baseline weekly average frequency is based on the subject's RP diary reporting daily symptomatic RP attack frequency for 5 to 30 days prior to the iloprost treatment. In embodiments, the baseline weekly average frequency is based on the subject's symptomatic RP diary reporting daily symptomatic RP attack frequency for 10 to 25 days prior to the iloprost treatment.

In embodiments, the weekly average frequency of symptomatic RP attacks after iloprost treatment is the weekly average of the number of symptomatic RP attack episodes in the subject after iloprost treatment. In embodiments, the weekly average frequency after iloprost treatment is based on the subject's RP diary reporting daily symptomatic RP attack frequency for at least 5 days after to the iloprost treatment. The subject can report the frequency of the daily symptomatic RP attacks at different times after the iloprost treatment, such as recording for at least 5 days directly following the treatment (week 1 after treatment) and/or recording for at least 5 days, 5 weeks after treatment. In embodiments, the subject reports the frequency of the daily symptomatic RP attacks from about 1 week to about 15 weeks after treatment, including all values therebetween. In embodiments, the subject reports the frequency of the daily symptomatic RP attacks from about 3 weeks to about 12 weeks after treatment, including all values therebetween. In embodiments, the subject reports the frequency of the daily symptomatic RP attacks up to about 9 weeks or up to about 12 weeks after treatment.

In embodiments, the weekly average frequency of symptomatic RP attacks is reduced by about 10% to about 90% from the baseline weekly average frequency, including all values therebetween. In embodiments, the weekly average frequency of symptomatic RP attacks is reduced by about 15% to about 60% from the baseline weekly average frequency, including all values therebetween. In embodiments, the weekly average frequency of symptomatic RP attacks is reduced by about 25% to about 55% from the baseline weekly average frequency, including all values therebetween. In embodiments, the weekly average frequency of symptomatic RP attacks is reduced by about 30% to about 50% from the baseline weekly average frequency, including all values therebetween. In embodiments, the weekly average frequency of symptomatic RP attacks is reduced by about 5%, about 10%, about 15%, about 20%, about 25%, about 30%, about 35%, about 40%, about 45%, about 50%, about 55%, about 60%, about 65%, or about 70% from the baseline weekly average frequency, including any values therebetween. In embodiments, the weekly average frequency of symptomatic RP attacks is reduced by at least about 5%, about 10%, about 15%, about 20%, about 25%, about 30%, about 35%, about 40%, about 45%, or about 50% from the baseline weekly average frequency, including any values therebetween. In embodiments, the weekly average frequency of symptomatic RP attacks is reduced by at least about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% from the baseline weekly average frequency, including any values therebetween. In one embodiment, the weekly average frequency reduction is provided as a mean reduction value for a SSc population treated with iloprost or a pharmaceutically acceptable salt or stereoisomer thereof.

In embodiments, a treatment effect of iloprost or a pharmaceutically acceptable salt or stereoisomer thereof on the weekly average frequency of symptomatic RP attacks is about −2.0 to about −30.0, including all values and subranges therebetween. In embodiments, a treatment effect of iloprost or a pharmaceutically acceptable salt or stereoisomer thereof on the weekly average frequency of symptomatic RP attacks is about −2.0 to about −15.0, including all values and subranges therebetween. In embodiments, a treatment effect of iloprost or a pharmaceutically acceptable salt or stereoisomer thereof on the weekly average frequency of symptomatic RP attacks is about −3.0 to about −10.0, including all values and subranges therebetween. In embodiments, a treatment effect of iloprost or a pharmaceutically acceptable salt or stereoisomer thereof on the weekly average frequency of symptomatic RP attacks is about −3.0 to about −8.0, including all values and subranges therebetween. In embodiments, a treatment effect of iloprost or a pharmaceutically acceptable salt or stereoisomer thereof on the weekly average frequency of symptomatic RP attacks is at least about −3.0, about −3.5, about −4.0, about −4.5, about −5.0, about −5.5, about −6.0, about −6.5, about −7.0, about −7.5, about −8.0, about −8.5, about −9.0, about −9.5, or about −10.0, including all values therebetween. A treatment effect is the change in the weekly average frequency of symptomatic RP attacks from baseline to end of efficacy follow-up. The primary analysis on this endpoint is performed based on an analysis of covariance (ANCOVA) model, including randomized treatment group and randomized stratification (i.e., use of phosphodiesterase inhibitors at screening) as factors and baseline weekly RP attacks as a covariate. The treatment comparisons (iloprost vs placebo) will be estimated together with the 95% confidence interval and p-value. For example, if the subject who received iloprost treatment and the subject who received placebo both had a baseline weekly average frequency of 30.0 attacks per week and the weekly average frequency of the subject who received iloprost reduced to 19.5 attacks and the subject who received placebo reduced to 25.0 attacks, then the treatment effect is-5.5 (in real life situations, the baseline values of iloprost and placebo groups would be expected to be different but the above example uses the same value for ease of explanation). The treatment effects are placebo corrected.

In embodiments, the weekly average frequency of symptomatic RP attacks is reduced for a period of about 1 week to about 6 months after one cycle of iloprost treatment, including any values therebetween. In embodiments, the weekly average frequency of symptomatic RP attacks is reduced for a period of about 2 weeks to about 3 months after one iloprost treatment, including any values therebetween. In embodiments, the weekly average frequency of symptomatic RP attacks is reduced for a period of about 2 weeks to about 15 weeks after one iloprost treatment, including any values therebetween. In embodiments, the weekly average frequency of symptomatic RP attacks is reduced for a period of about 3 weeks to about 8 weeks after one iloprost treatment, including any values therebetween. In embodiments, the weekly average frequency of symptomatic RP attacks is reduced for a period of at least 2 week, 3 weeks, 4 weeks, 5 weeks, 6 weeks, 7 weeks, 8 weeks, 9 weeks, 10 weeks, 11 weeks, or 12 weeks after one iloprost treatment, including any values therebetween. In embodiments, the weekly average frequency of symptomatic RP attacks is reduced for a period of about 7 weeks, about 8 weeks, about 9 weeks, or about 10 weeks after one iloprost treatment, including any values therebetween.

The present disclosure also relates to reducing the weekly average duration of symptomatic RP attacks from baseline in a SSc subject experiencing symptomatic attacks, comprising administering iloprost or a pharmaceutically acceptable salt thereof or a stereoisomer thereof. In embodiments, iloprost or a pharmaceutically acceptable salt thereof or a stereoisomer thereof is administered in any method as disclosed herein.

In embodiments, the weekly average duration of symptomatic RP attacks is determined according to the subject's RP diary. The baseline weekly average duration of symptomatic RP attacks is the weekly average of the total duration of all symptomatic RP attack episodes in the subject before iloprost treatment. In embodiments, the baseline weekly average duration is based on the subject's RP diary reporting daily symptomatic RP attack duration for each symptomatic RP attack episode for 5 to 30 days prior to the iloprost treatment. In embodiments, the baseline weekly average duration is based on the subject's RP diary reporting daily symptomatic RP attack duration for each symptomatic RP attack episode for 10 to 25 days prior to the iloprost treatment.

In embodiments, the weekly average duration of symptomatic RP attacks after iloprost treatment is the weekly average of the total duration of all symptomatic RP attack episodes in the subject after iloprost treatment. In embodiments, the weekly average duration after iloprost treatment is based on the subject's RP diary reporting daily symptomatic RP attack duration for each symptomatic RP attack episode for at least 5 days after to the iloprost treatment. The subject can report daily symptomatic RP attack duration for each symptomatic RP attack episode at different times after the iloprost treatment, such as recording for at least 5 days directly following the treatment (week 1 after treatment) and/or recording for at least 5 days, 6 weeks after treatment. In embodiments, the subject reports the duration of each symptomatic RP attack episode from about 1 week to about 15 weeks after treatment, including all values therebetween. In embodiments, the subject reports the duration of each symptomatic RP attack episode from about 3 weeks to about 12 weeks after treatment, including all values therebetween. In embodiments, the subject reports the duration of each symptomatic RP attack episode up to about 9 weeks or up to about 12 weeks after treatment.

In embodiments, the weekly average duration of symptomatic RP attacks is reduced by about 10% to about 90% from the baseline weekly average duration, including all values therebetween. In embodiments, the weekly average duration of symptomatic RP attacks is reduced by about 15% to about 60% from the baseline weekly average duration, including all values therebetween. In embodiments, the weekly average duration of symptomatic RP attacks is reduced by about 15% to about 55% from the baseline weekly average duration, including all values therebetween. In embodiments, the weekly average duration of symptomatic RP attacks is reduced by about 5%, about 10%, about 15%, about 20%, about 25%, about 30%, about 35%, about 40%, about 45%, about 50%, about 55%, about 60%, about 65%, or about 70% from the baseline weekly average duration, including any values therebetween. In embodiments, the weekly average duration of symptomatic RP attacks is reduced by at least about 5%, about 10%, about 15%, about 20%, about 25%, about 30%, about 35%, about 40%, about 45%, or about 50% from the baseline weekly average duration, including any values therebetween. In embodiments, the weekly average duration of symptomatic RP attacks is reduced by at least about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% from the baseline weekly average duration, including any values therebetween. In one embodiment, the weekly average duration reduction is provided as a mean % reduction value for a SSc population treated with iloprost or a pharmaceutically acceptable salt or stereoisomer thereof.

In embodiments, the weekly average duration of symptomatic RP attacks is reduced by about 30 minutes to about 2000 minutes, including all values and subranges therebetween. In embodiments, the weekly average duration of symptomatic RP attacks is reduced by about 30 minutes to about 1000 minutes, including all values and subranges therebetween. In embodiments, the weekly average duration of symptomatic RP attacks is reduced by about 30 minutes to about 500 minutes, including all values and subranges therebetween. In embodiments, the weekly average duration of symptomatic RP attacks is reduced by about 45 minutes to about 400 minutes, including all values and subranges therebetween. In embodiments, the weekly average duration of symptomatic RP attacks is reduced by about 45 minutes to about 300 minutes, including all values and subranges therebetween. In embodiments, the weekly average duration of symptomatic RP attacks is reduced by about 60 minutes to about 250 minutes, including all values and subranges therebetween. In embodiments, the weekly average duration of symptomatic RP attacks is reduced by at least about 30 minutes, about 45 minutes, about 60 minutes, about 75 minutes, about 90 minutes, about 105 minutes, about 120 minutes, about 135 minutes, about 150 minutes, about 165 minutes, about 180 minutes, about 195 minutes, about 210 minutes, about 225 minutes, about 240 minutes, about 250 minutes, about 265 minutes, about 285 minutes, or about 300 minutes, including all values therebetween. In one embodiment, the weekly average duration reduction is provided as a mean value in minutes for a SSc population treated with iloprost or a pharmaceutically acceptable salt thereof.

In embodiments, the mean reduction of weekly average duration of symptomatic RP attacks in a SSc population is by about 30 minutes to about 500 minutes, including all values and subranges therebetween.

In embodiments, a treatment effect of iloprost or a pharmaceutically acceptable salt or stereoisomer thereof on the weekly average duration of symptomatic RP attacks is about −30 minutes to about −2000 minutes, including all values and subranges therebetween. In embodiments, a treatment effect of iloprost or a pharmaceutically acceptable salt or stereoisomer thereof on the weekly average duration of symptomatic RP attacks is about −30 minutes to about −1000 minutes, including all values and subranges therebetween. In embodiments, a treatment effect of iloprost or a pharmaceutically acceptable salt or stereoisomer thereof on the weekly average duration of symptomatic RP attacks is about −30 minutes to about −500 minutes, including all values and subranges therebetween. In embodiments, a treatment effect of iloprost or a pharmaceutically acceptable salt or stereoisomer thereof on the weekly average duration of symptomatic RP attacks is about −45 minutes to about −400 minutes, including all values and subranges therebetween. In embodiments, a treatment effect of iloprost or a pharmaceutically acceptable salt or stereoisomer thereof on the weekly average duration of symptomatic RP attacks is about −45 minutes to about −300 minutes, including all values and subranges therebetween. In embodiments, a treatment effect of iloprost or a pharmaceutically acceptable salt or stereoisomer thereof on the weekly average duration of symptomatic RP attacks is about −60 minutes to about −150 minutes, including all values and subranges therebetween. In embodiments, a treatment effect of iloprost or a pharmaceutically acceptable salt or stereoisomer thereof on the weekly average duration of symptomatic RP attacks is at least about −30 minutes, about −45 minutes, about −60 minutes, about −75 minutes, about −90 minutes, about −105 minutes, about −120 minutes, about −135 minutes, about −150 minutes, about −165 minutes, about −180 minutes, about −195 minutes, about −210 minutes, about −225 minutes, about −240 minutes, about −250 minutes, about −265 minutes, about −285 minutes, or about −300 minutes, including all values therebetween. A treatment effect is the change in the weekly average duration of symptomatic RP attacks from baseline to end of efficacy follow-up. The primary analysis on this endpoint is performed based on an analysis of covariance (ANCOVA) model, including randomized treatment group and randomized stratification (i.e., use of phosphodiesterase inhibitors at screening) as factors and baseline weekly RP attacks as a covariate. The treatment comparisons (iloprost vs placebo) will be estimated together with the 95% confidence interval and p-value. For example, if the subject who received iloprost treatment and the subject who received placebo both had a baseline weekly average duration of 350 minutes per week and the subject who received placebo reduced to 450 minutes per week, then the treatment effect is-100 minutes.

In embodiments, a mean treatment effect of iloprost or a pharmaceutically acceptable salt or stereoisomer thereof on the weekly average duration of symptomatic RP attacks in a SSc population is about −30 minutes to about −500 minutes, including all values and subranges therebetween.

In embodiments, the weekly average duration of symptomatic RP attacks is reduced for a period of about 1 week to about 6 months after one cycle of iloprost treatment, including any values therebetween. In embodiments, the weekly average duration of symptomatic RP attacks is reduced for a period of about 2 weeks to about 3 months after one iloprost treatment, including any values therebetween. In embodiments, the weekly average duration of symptomatic RP attacks is reduced for a period of about 3 weeks to about 15 weeks after one iloprost treatment, including any values therebetween. In embodiments, the weekly average duration of symptomatic RP attacks is reduced for a period of about 2 weeks to about 8 weeks after one iloprost treatment, including any values therebetween. In embodiments, the weekly average duration of symptomatic RP attacks is reduced for a period of at least 2 week, 3 weeks, 4 weeks, 5 weeks, 6 weeks, 7 weeks, 8 weeks, 9 weeks, 10 weeks, 11 weeks, or 12 weeks after one iloprost treatment, including any values therebetween. In embodiments, the weekly average duration of symptomatic RP attacks is reduced for a period of about 7 weeks, about 8 weeks, about 9 weeks, or about 10 weeks after one iloprost treatment, including any values therebetween.

The present disclosure also relates to reducing the weekly average severity of symptomatic RP attacks from baseline in a SSc subject experiencing symptomatic attacks, comprising administering iloprost or a pharmaceutically acceptable salt thereof or a stereoisomer thereof. In embodiments, iloprost or a pharmaceutically acceptable salt thereof or a stereoisomer thereof is administered in any method as disclosed herein.

In embodiments, the severity of symptomatic RP attacks is measured by a symptom of the fingers with a worst baseline weekly average score selected from pain, numbness, discomfort, or tingling, based on a numeric rating scale (severity score). In embodiments, the severity is measured by different symptom based on what the subject reports as the worst symptom before iloprost treatment. In embodiments, if the baseline weekly average severity score is the same value for two symptoms of the fingers, the baseline weekly average will be based on the following order of rank: pain>numbness>tingling>discomfort.

In embodiments, the measuring the severity of symptomatic RP attacks by the subject's worst symptoms of pain, numbness, discomfort, or tingling, allows for some individualized measurement because every subject experiences symptomatic RP attacks differently. By focusing on the subject's worst symptoms, in embodiments, a subject's progress or status in his or her symptomatic RP attacks can be monitored better than asking the subject to give one severity score considering all the symptoms he or she experienced in a particular day.

In embodiments, the weekly average severity of symptomatic RP attacks is determined according to the subject's RP diary. The baseline weekly average severity of symptomatic RP attacks is the weekly average of the severity score of a symptom of symptomatic RP attack episodes in the subject before iloprost treatment. In embodiments, the baseline weekly average severity is based on the subject's RP diary reporting daily severity score for each symptom of the fingers during symptomatic RP attacks (pain, numbness, tingling, and discomfort) for 5 to 30 days prior to the iloprost treatment. In embodiments, the baseline weekly average severity is based on the subject's RP diary reporting daily severity score for each symptom of the fingers during symptomatic RP attacks for 10 to 25 days prior to the iloprost treatment.

In embodiments, the weekly average severity of symptomatic RP attacks after iloprost treatment is the weekly average of the severity score of a symptom of symptomatic RP attack episodes in the subject after iloprost treatment. In embodiments, the weekly average severity after iloprost treatment is based on the subject's RP diary reporting daily severity score for each symptoms of the fingers for at least 5 days after to the iloprost treatment. The subject can report daily symptomatic RP attack severity score for each symptom of the fingers at different times after the iloprost treatment, such as recording for at least 5 days directly following the treatment (week 1 after treatment) and/or recording for at least 5 days, 7 weeks after treatment. In embodiments, the subject reports the symptomatic RP attack severity score for each symptom of the fingers from about 1 week to about 15 weeks after treatment, including all values therebetween. In embodiments, the subject reports the symptomatic RP attack severity score for each symptom of the fingers from about 3 weeks to about 12 weeks after treatment, including all values therebetween. In embodiments, the subject reports the symptomatic RP attack severity score for each symptom of the fingers up to about 9 weeks or up to about 12 weeks after treatment.

In embodiments, when assessing whether the iloprost treatment resulted in reduction of the weekly average severity of the symptomatic RP attacks, the weekly average of the symptom determined to be the worst in the subject is compared to the weekly average severity score for the same symptom after iloprost treatment. That is, if a subject reported pain as the worst symptom prior to iloprost treatment (i.e., pain had the highest severity score baseline weekly average compared to numbness, tingling, or discomfort), then the subject's weekly average of pain severity scores after iloprost treatment will be compared to the baseline weekly average severity score for pain. This method allows individualization of outcome measurements as different subject experience the symptomatic RP attack symptoms in different degrees.

In embodiments, the weekly average severity score of symptomatic RP attacks is reduced by about 10% to about 90% from the baseline weekly average severity score, including all values therebetween. In embodiments, the weekly average severity score of symptomatic RP attacks is reduced by about 15% to about 60% from the baseline weekly average severity score, including all values therebetween. In embodiments, the weekly average severity score of symptomatic RP attacks is reduced by about 20% to about 50% from the baseline weekly average severity score, including all values therebetween. In embodiments, the weekly average severity score of symptomatic RP attacks is reduced by about 5%, about 10%, about 15%, about 20%, about 25%, about 30%, about 35%, about 40%, about 45%, about 50%, about 55%, about 60%, about 65%, or about 70%, from the baseline weekly average severity score, including all values therebetween. In embodiments, the weekly average severity score of symptomatic RP attacks is reduced by at least about 5%, about 10%, about 15%, about 20%, about 25%, about 30%, about 35%, about 40%, about 45%, or about 50% from the baseline weekly average severity score, including all values therebetween. In embodiments, the weekly average duration of symptomatic RP attacks is reduced by at least about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% from the baseline weekly average severity score, including any values therebetween. In one embodiment, the weekly average severity score reduction is provided as a mean % reduction value for a SSc population treated with iloprost or a pharmaceutically acceptable salt or stereoisomer thereof.

In embodiments, the weekly average severity score of symptomatic RP attacks is reduced by a number in the range of about 0.2 to about 5.0 from the baseline weekly average severity score, wherein the severity score is based on 0 to 10 numeric rating scale. In embodiments, the weekly average severity score of symptomatic RP attacks is reduced by a number in the range of about 0.3 to about 4.0 from the baseline weekly average severity score, wherein the severity score is based on 0 to 10 numeric rating scale. In embodiments, the weekly average severity score of symptomatic RP attacks is reduced by a number in the range of about 0.5 to about 3.0 from the baseline weekly average severity score, wherein the severity score is based on 0 to 10 numeric rating scale. In embodiments, the weekly average severity score of symptomatic RP attacks is reduced by a number in the range of about 0.5 to about 3.0 from the baseline weekly average severity score, wherein the severity score is based on 0 to 10 numeric rating scale. In embodiments, the weekly average severity score of symptomatic RP attacks is reduced by a number in the range of about 0.5 to about 2.0 from the baseline weekly average severity score, wherein the severity score is based on 0 to 10 numeric rating scale. In embodiments, the weekly average severity score of symptomatic RP attacks is reduced by a number in the range of about 0.5 to about 1.5 from the baseline weekly average severity score, wherein the severity score is based on 0 to 10 numeric rating scale. In embodiments, the weekly average severity score of symptomatic RP attacks is reduced by a number in the range of about 0.6 to about 1.3 from the baseline weekly average severity score, wherein the severity score is based on 0 to 10 numeric rating scale. In embodiments, the weekly average severity score of symptomatic RP attacks is reduced by at least about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 from the baseline weekly average severity score, wherein the severity score is based on 0 to 10 numeric rating scale. In one embodiment, the weekly average severity score reduction is provided as a mean value (score) for a SSc population treated with iloprost or a pharmaceutically acceptable salt or stereoisomer thereof.

In embodiments, a treatment effect of iloprost or a pharmaceutically acceptable salt or stereoisomer thereof on the weekly average severity score of symptomatic RP attacks is about −0.2 to about −5.0, including all values and subranges therebetween. In embodiments, a treatment effect of iloprost or a pharmaceutically acceptable salt or stereoisomer thereof on the weekly average severity score of symptomatic RP attacks is about −0.3 to about −3.0, including all values and subranges therebetween. In embodiments, a treatment effect of iloprost or a pharmaceutically acceptable salt or stereoisomer thereof on the weekly average severity score of symptomatic RP attacks is about −0.5 to about −2.5, including all values and subranges therebetween. In embodiments, a treatment effect of iloprost or a pharmaceutically acceptable salt or stereoisomer thereof on the weekly average severity score of symptomatic RP attacks is about −0.3 to about −2.0, including all values and subranges therebetween. In embodiments, a treatment effect of iloprost or a pharmaceutically acceptable salt or stereoisomer thereof on the weekly average severity score of symptomatic RP attacks is about −0.5 to about −1.5, including all values and subranges therebetween. In embodiments, a treatment effect of iloprost or a pharmaceutically acceptable salt or stereoisomer thereof on the weekly average severity score of symptomatic RP attacks is about −0.6 to about −1.5, including all values and subranges therebetween. In embodiments, a treatment effect of iloprost or a pharmaceutically acceptable salt or stereoisomer thereof on the weekly average severity score of symptomatic RP attacks is at least about −0.2, about −0.3, about −0.4, about −0.5, about −0.6, about −0.7, about −0.8, about −0.9, about −1.0, about −1.1, about −1.2, about −1.3, about −1.4, about −1.5, about −1.6, about −1.7, about −1.8, about −1.9, or about −2.0, including all values therebetween. A treatment effect is the change in the weekly average severity score of symptomatic RP attacks from baseline to end of efficacy follow-up. The primary analysis on this endpoint is performed based on an analysis of covariance (ANCOVA) model, including randomized treatment group and randomized stratification (i.e., use of phosphodiesterase inhibitors at screening) as factors and baseline weekly RP attacks as a covariate. The treatment comparisons (iloprost vs placebo) will be estimated together with the 95% confidence interval and p-value. For example, if the subject who received iloprost treatment and the subject who received placebo both had a baseline weekly average severity score of 5.5 and the weekly average severity score of the subject who received iloprost reduced to 4.0 and the subject who received placebo reduced to 5.0, then the treatment effect is-1.0.

In embodiments, the weekly average severity of symptomatic RP attacks is reduced for a period of about 1 week to about 6 months after one cycle of iloprost treatment, including any values therebetween. In embodiments, the weekly average severity of symptomatic RP attacks is reduced for a period of about 2 weeks to about 3 months after one iloprost treatment, including any values therebetween. In embodiments, the weekly average severity of symptomatic RP attacks is reduced for a period of about 3 weeks to about 20 weeks after one iloprost treatment, including any values therebetween. In embodiments, the weekly average severity of symptomatic RP attacks is reduced for a period of at least 2 week, 3 weeks, 4 weeks, 5 weeks, 6 weeks, 7 weeks, 8 weeks, 9 weeks, 10 weeks, 11 weeks, or 12 weeks after one iloprost treatment, including any values therebetween. In embodiments, the weekly average severity of symptomatic RP attacks is reduced for a period of about 7 weeks, about 8 weeks, about 9 weeks, or about 10 weeks after one iloprost treatment, including any values therebetween.

The present disclosure also relates to reducing the weekly average frequency and weekly average severity of symptomatic RP attacks from baseline in a SSc subject experiencing symptomatic attacks, comprising administering any one of the composition comprising iloprost or a pharmaceutically acceptable salt thereof or a stereoisomer thereof, as disclosed herein.

The present disclosure also relates to reducing the weekly average duration and weekly average severity of symptomatic RP attacks from baseline in a SSc subject experiencing symptomatic attacks, comprising administering any one of the composition comprising iloprost or a pharmaceutically acceptable salt thereof or a stereoisomer thereof, as disclosed herein. In embodiments the weekly average duration is reduced by at least 30% and the weekly average severity is reduced by at least 30%. In embodiments the weekly average duration is reduced by at least 40% and the weekly average severity is reduced by at least 40%. In embodiments the weekly average duration is reduced by at least 50% and the weekly average severity is reduced by at least 50%.

The present disclosure also relates to reducing the weekly average frequency and weekly average duration of symptomatic RP attacks from baseline in a SSc subject experiencing symptomatic attacks, comprising administering any one of the composition comprising iloprost or a pharmaceutically acceptable salt thereof or a stereoisomer thereof, as disclosed herein.

The present disclosure also relates to reducing the weekly average frequency, weekly average duration, and weekly average severity of symptomatic RP attacks from baseline in a SSc subject experiencing symptomatic attacks, comprising administering any one of the composition comprising iloprost or a pharmaceutically acceptable salt thereof or a stereoisomer thereof, as disclosed herein.

The present disclosure also relates to increasing the number of days without symptomatic RP attacks in a SSc subject from baseline, comprising administering any one of the compositions comprising iloprost or a pharmaceutically acceptable salt thereof or a stereoisomer thereof, as disclosed herein.

The present disclosure also relates to reducing the worst pain associated with symptomatic RP attacks from baseline in a SSc subject, comprising administering any one of the compositions comprising iloprost or a pharmaceutically acceptable salt thereof or a stereoisomer thereof, as disclosed herein. In embodiments, the worst pain is provided as a score given to the worst pain in a given day. In embodiments, the worst pain score is the weekly average worst pain score. In embodiments, the weekly average worst pain score is reduced by a number in the range of about 0.2 to about 5.0 from the baseline weekly average worst pain score, wherein the worst pain score is based on 0 to 10 numeric rating scale. In embodiments, the weekly average worst pain score of symptomatic RP attacks is reduced by a number in the range of about 0.3 to about 4.0 from the baseline weekly average worst pain score, wherein the worst pain score is based on 0 to 10 numeric rating scale. In embodiments, the weekly average worst pain score of symptomatic RP attacks is reduced by a number in the range of about 0.5 to about 3.0 from the baseline weekly average worst pain score, wherein the pain score is based on 0 to 10 numeric rating scale. In embodiments, the weekly average worst pain score of symptomatic RP attacks is reduced by a number in the range of about 0.5 to about 2.0 from the baseline weekly average worst pain score, wherein the pain score is based on 0 to 10 numeric rating scale. In embodiments, the weekly average worst pain score of symptomatic RP attacks is reduced by a number in the range of about 0.5 to about 1.5 from the baseline weekly average worst pain score, wherein the pain score is based on 0 to 10 numeric rating scale. In embodiments, the weekly average worst pain score of symptomatic RP attacks is reduced by a number in the range of about 0.6 to about 1.3 from the baseline weekly average worst pain score, wherein the pain score is based on 0 to 10 numeric rating scale. In embodiments, the weekly average worst pain score of symptomatic RP attacks is reduced by at least about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 from the baseline weekly average worst pain score, wherein the worst pain score is based on 0 to 10 numeric rating scale.

The present disclosure also relates to reducing the worst numbness associated with symptomatic RP attacks from baseline in a SSc subject, comprising administering any one of the compositions comprising iloprost or a pharmaceutically acceptable salt thereof or a stereoisomer thereof, as disclosed herein. In embodiments, the worst numbness is provided as a score given to the worst numbness in a given day. In embodiments, the worst numbness score is the weekly average worst numbness score. In embodiments, the weekly average worst numbness score is reduced by a number in the range of about 0.2 to about 5.0 from the baseline weekly average worst numbness score, wherein the worst numbness score is based on 0 to 10 numeric rating scale. In embodiments, the weekly average worst numbness score of symptomatic RP attacks is reduced by a number in the range of about 0.3 to about 4.0 from the baseline weekly average worst numbness score, wherein the worst numbness score is based on 0 to 10 numeric rating scale. In embodiments, the weekly average worst numbness score of symptomatic RP attacks is reduced by a number in the range of about 0.5 to about 2.0 from the baseline weekly average worst numbness score, wherein the numbness score is based on 0 to 10 numeric rating scale. In embodiments, the weekly average worst numbness score of symptomatic RP attacks is reduced by a number in the range of about 0.7 to about 1.2 from the baseline weekly average worst numbness score, wherein the numbness score is based on 0 to 10 numeric rating scale. In embodiments, the weekly average worst numbness score of symptomatic RP attacks is reduced by a number in the range of about 0.5 to about 3.0 from the baseline weekly average worst numbness score, wherein the numbness score is based on 0 to 10 numeric rating scale. In embodiments, the weekly average worst numbness score of symptomatic RP attacks is reduced by at least about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 from the baseline weekly average worst numbness score, wherein the worst numbness score is based on 0 to 10 numeric rating scale.

The present disclosure also relates to reducing the worst tingling associated with symptomatic RP attacks from baseline in a SSc subject, comprising administering any one of the compositions comprising iloprost or a pharmaceutically acceptable salt thereof or a stereoisomer thereof, as disclosed herein. In embodiments, the worst tingling is provided as a score given to the worst tingling in a given day. In embodiments, the worst tingling score is the weekly average worst tingling score. In embodiments, the weekly average worst tingling score is reduced by a number in the range of about 0.2 to about 5.0 from the baseline weekly average worst tingling score, wherein the worst tingling score is based on 0 to 10 numeric rating scale. In embodiments, the weekly average worst tingling score of symptomatic RP attacks is reduced by a number in the range of about 0.3 to about 4 0 from the baseline weekly average worst tingling score, wherein the worst tingling score is based on 0 to 10 numeric rating scale. In embodiments, the weekly average worst tingling score of symptomatic RP attacks is reduced by a number in the range of about 0.5 to about 3.0 from the baseline weekly average worst tingling, wherein the tingling score is based on 0 to 10 numeric rating scale. In embodiments, the weekly average worst tingling score of symptomatic RP attacks is reduced by a number in the range of about 0.5 to about 2.0 from the baseline weekly average worst tingling, wherein the tingling score is based on 0 to 10 numeric rating scale. In embodiments, the weekly average worst tingling score of symptomatic RP attacks is reduced by a number in the range of about 0.7 to about 1.2 from the baseline weekly average worst tingling, wherein the tingling score is based on 0 to 10 numeric rating scale. In embodiments, the weekly average worst tingling score of symptomatic RP attacks is reduced by at least about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 from the baseline weekly average worst tingling score, wherein the worst tingling score is based on 0 to 10 numeric rating scale.

The present disclosure also relates to reducing the worst discomfort associated with symptomatic RP attacks rom baseline in a SSc subject, comprising administering any one of the compositions comprising iloprost or a pharmaceutically acceptable salt thereof or a stereoisomer thereof, as disclosed herein. In embodiments, the worst discomfort is provided as a score given to the worst discomfort in a given day. In embodiments, the worst discomfort score is the weekly average worst discomfort score. In embodiments, the weekly average worst discomfort score is reduced by a number in the range of about 0.2 to about 5.0 from the baseline weekly average worst discomfort score, wherein the worst discomfort score is based on 0 to 10 numeric rating scale. In embodiments, the weekly average worst discomfort score of symptomatic RP attacks is reduced by a number in the range of about 0.3 to about 4.0 from the baseline weekly average worst discomfort score, wherein the worst discomfort score is based on 0 to 10 numeric rating scale. In embodiments, the weekly average worst discomfort score of symptomatic RP attacks is reduced by a number in the range of about 0.5 to about 3.0 from the baseline weekly average worst discomfort, wherein the discomfort score is based on 0 to 10 numeric rating scale. In embodiments, the weekly average worst discomfort score of symptomatic RP attacks is reduced by a number in the range of about 0.5 to about 2.0 from the baseline weekly average worst discomfort, wherein the discomfort score is based on 0 to 10 numeric rating scale. In embodiments, the weekly average worst discomfort score of symptomatic RP attacks is reduced by a number in the range of about 0.7 to about 1.2 from the baseline weekly average worst discomfort, wherein the discomfort score is based on 0 to 10 numeric rating scale. In embodiments, the weekly average worst discomfort score of symptomatic RP attacks is reduced by at least about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 from the baseline weekly average worst discomfort score, wherein the worst discomfort score is based on 0 to 10 numeric rating scale.

The present disclosure also relates to reducing the average duration of a symptomatic RP attack episode from baseline in a SSc subject, comprising administering any one of the compositions comprising iloprost or a pharmaceutically acceptable salt thereof or a stereoisomer thereof, as disclosed herein.

The present disclosure also relates to changing a SSc subject's assessment of overall change in symptomatic RP attacks from baseline, comprising administering any one of the compositions comprising iloprost or a pharmaceutically acceptable salt thereof or a stereoisomer thereof, as disclosed herein.

The present disclosure also relates to changing a SSc subject's assessment of overall severity in symptomatic RP attacks from baseline, comprising administering any one of the compositions comprising iloprost or a pharmaceutically acceptable salt thereof or a stereoisomer thereof, as disclosed herein.

The present disclosure also relates to restoring function lost due to SSc, comprising administering any one of the compositions comprising iloprost or a pharmaceutically acceptable salt thereof or a stereoisomer thereof, as disclosed herein. In embodiments, iloprost or a pharmaceutically acceptable salt thereof or a stereoisomer thereof can enhance cutaneous blood flow. In embodiments, iloprost or a pharmaceutically acceptable salt thereof or a stereoisomer thereof can reduce microvascular inflammation. In embodiments, iloprost or a pharmaceutically acceptable salt thereof or a stereoisomer thereof can attenuate fibrosis. In embodiments, iloprost or a pharmaceutically acceptable salt thereof or a stereoisomer thereof can decrease platelet aggregation and adhesion.

The present disclosure also relates to reducing the severity of vasconstrictive episodes response in a SSc subject, comprising administering any one of the compositions comprising iloprost or a pharmaceutically acceptable salt thereof or a stereoisomer thereof, as disclosed herein.

The present disclosure also relates to methods of treating digital ischemia exacerbation in a SSc subject, comprising administering any one of the compositions comprising iloprost or a pharmaceutically acceptable salt thereof or a stereoisomer thereof, as disclosed herein. The present disclosure also relates to reducing the frequency of having digital ischemia exacerbation episodes in a SSc subject, comprising administering any one of the compositions comprising iloprost or a pharmaceutically acceptable salt thereof or a stereoisomer thereof, as disclosed herein.

The present disclosure also relates to preventing the development of digital ischemia in a SSc subject, comprising administering any one of the compositions comprising iloprost or a pharmaceutically acceptable salt thereof or a stereoisomer thereof, as disclosed herein. The present disclosure also relates to reducing the frequency of developing digital ischemia in a SSc subject, comprising administering any one of the compositions comprising iloprost or a pharmaceutically acceptable salt thereof or a stereoisomer thereof, as disclosed herein.

The present disclosure also relates to preventing the development of critical digital ischemia in a SSc subject, comprising administering any one of the compositions comprising iloprost or a pharmaceutically acceptable salt thereof or a stereoisomer thereof, as disclosed herein. The present disclosure also relates to reducing the frequency of developing critical digital ischemia in a SSc subject, comprising administering any one of the compositions comprising iloprost or a pharmaceutically acceptable salt thereof or a stereoisomer thereof, as disclosed herein.

The present disclosure also relates to preventing the development of digital ischemic episodes in a SSc subject, comprising administering any one of the compositions comprising iloprost or a pharmaceutically acceptable salt thereof or a stereoisomer thereof, as disclosed herein. The present disclosure also relates to reducing the frequency of developing digital ischemic episodes in a SSc subject, comprising administering any one of the compositions comprising iloprost or a pharmaceutically acceptable salt thereof or a stereoisomer thereof, as disclosed herein.

The present disclosure also relates to preventing the development of digital ischemic ulcers in a SSc subject, comprising administering any one of the compositions comprising iloprost or a pharmaceutically acceptable salt thereof or a stereoisomer thereof, as disclosed herein. The present disclosure also relates to reducing the frequency of developing digital ischemic ulcers in a SSc subject, comprising administering any one of the compositions comprising iloprost or a pharmaceutically acceptable salt thereof or a stereoisomer thereof, as disclosed herein.

The present disclosure also relates to preventing the development of digital ischemic lesions in a SSc subject, comprising administering any one of the compositions comprising iloprost or a pharmaceutically acceptable salt thereof or a stereoisomer thereof, as disclosed herein. The present disclosure also relates to reducing the frequency of developing digital ischemic lesions in a SSc subject, comprising administering any one of the compositions comprising iloprost or a pharmaceutically acceptable salt thereof or a stereoisomer thereof, as disclosed herein.

The present disclosure also relates to preventing the development of symptomatic ischemic lesions in a SSc subject, comprising administering any one of the compositions comprising iloprost or a pharmaceutically acceptable salt thereof or a stereoisomer thereof, as disclosed herein. The present disclosure also relates to reducing the frequency of developing symptomatic ischemic lesions in a SSc subject, comprising administering any one of the compositions comprising iloprost or a pharmaceutically acceptable salt thereof or a stereoisomer thereof, as disclosed herein.

The present disclosure also relates to preventing the development of gangrene in a SSc subject, comprising administering any one of the compositions comprising iloprost or a pharmaceutically acceptable salt thereof or a stereoisomer thereof, as disclosed herein. The present disclosure also relates to preventing the frequency of developing gangrene in a SSc subject, comprising administering any one of the compositions comprising iloprost or a pharmaceutically acceptable salt thereof or a stereoisomer thereof, as disclosed herein.

The present disclosure also relates to preventing the development of digital infection in a SSc subject, comprising administering any one of the compositions comprising iloprost or a pharmaceutically acceptable salt thereof or a stereoisomer thereof, as disclosed herein. The present disclosure also relates to preventing the frequency of developing digital infection in a SSc subject, comprising administering any one of the compositions comprising iloprost or a pharmaceutically acceptable salt thereof or a stereoisomer thereof, as disclosed herein.

In embodiments, any of the aforementioned benefits or effects of iloprost or a pharmaceutically acceptable salt thereof can last about 3 weeks to about 12 weeks after a single treatment, including all ranges and values therebetween. In embodiments, any of the aforementioned benefits or effects of iloprost or a pharmaceutically acceptable salt thereof can, on average in a SSc population, last about 3 weeks to about 12 weeks after a single treatment, including all ranges and values therebetween. The benefit or effects of iloprost or a pharmaceutically acceptable salt thereof can be extended or reduced depending on number of factors. The symptomatic RP attack episodes generally worsen in the winter and/or during times of emotional or physical stress.

### EXAMPLES

The disclosure now being generally described, it will be more readily understood by reference to the following examples which are included merely for purposes of illustration of certain aspects and embodiments of the present invention, and are not intended to limit the invention.

### Example 1. A Multicenter, Double-Blind, Randomized, Placebo-Controlled, Study Evaluating Intravenous Iloprost in Subjects with Symptomatic Raynaud's Phenomenon Secondary to Systemic Sclerosis

This was a multicenter, double-blind, randomized, placebo-controlled study to provide an initial evaluation of the effect of iloprost on the symptomatic relief of RP attacks in subjects with SSc. As a pilot study, it was not powered to provide a definitive estimate of the effect of iloprost. Importantly, this study tested the feasibility of collecting the study endpoints and logistics of study operations that included, among other things, an electronic patient-reported outcomes (ePRO) diary for the evaluation of subject response to the treatment, as well as the infusion workflow for this multiday treatment. Subjects were allowed to continue receiving stable standard of care therapies for the management of symptomatic RP (e.g., calcium channel blockers, angiotensin-converting enzyme inhibitors, statins, fluoxetine, and low-dose acetylsalicylic acid) or may have participated without the use of background standard of care therapies.

The primary objective was to evaluate the efficacy of iloprost compared to placebo on the change in the weekly frequency of symptomatic Raynaud's Phenomenon (RP) attacks from baseline in subjects with symptomatic RP secondary to Systemic Sclerosis (SSc). The exploratory objectives were the following:
To evaluate the efficacy of iloprost compared to placebo on the severity of RP attack symptoms
To evaluate the efficacy of iloprost compared to placebo on the Raynaud's Condition Score
To evaluate the efficacy of iloprost compared to placebo on hand function as assessed by the Cochin Hand Function Scale (CHFS)
To evaluate the safety and tolerability of iloprost
To evaluate the efficacy of iloprost compared to placebo on symptomatic RP attack duration
To evaluate the efficacy of iloprost compared to placebo on the worst pain associated with symptomatic RP
To evaluate the efficacy of iloprost compared to placebo on the subject assessment of overall improvement in symptomatic RP
To evaluate the pharmacokinetics (PK) of iloprost in subjects with symptomatic RP secondary to SSc

### Methodology:

The study consisted of an up to 30-day screening period during which subjects completed a daily ePRO diary to record information regarding all symptomatic RP attacks (e.g., severity of symptoms, duration, and hand function). The up to 30-day screening period consisted of a 5-day eligibility period and an up to 25-day baseline ePRO diary completion period:
During the 5-day eligibility period, eligible subjects must have had a minimum of 10 symptomatic RP attacks, documented in the ePRO diary, that occurred over at least 3 separate days of the 5-day eligibility period.
During the baseline ePRO diary completion period, eligible subjects completed the daily ePRO diary for a minimum of 10 days (up to a maximum of 25 days) until the day of randomization. The baseline ePRO diary completion period may have been extended beyond 10 days in order to accommodate the preferred Monday through Friday dosing schedule. The ePRO symptomatic RP attack data from this time period were used for the calculation of the baseline frequency of symptomatic RP attacks.

Eligible subjects were given the option to participate in a PK sub-study. Subjects who participated in the sub-study provided plasma samples for PK analysis. The samples were analyzed for iloprost concentrations using validated liquid chromatography mass spectrometry methods.

Subjects were randomized in a 1:1 ratio to iloprost injection for IV use or placebo. Randomization was stratified based on the use of phosphodiesterase inhibitors at screening. Study drug administration began on Day 1, and subjects received study drug for 5 consecutive days (e.g., Monday through Friday) as an IV infusion over 6 hours each day via a peripheral line (NovaCath™ Integrated IV Catheter System) or a peripherally inserted central catheter using an infusion pump.

Subjects must have had a systolic blood pressure ≥85 mmHg (sitting position) prior to study drug administration each day of administration. On Day 1, study drug was initiated at a starting dose of 0.5 ng/kg/min, and dose increases occurred every 30 minutes (±5 minutes) in increments of 0.5 ng/kg/min up to 2.0 ng/kg/min or the individual tolerated dose. If dose-limiting adverse events (e.g., headache, flushing, jaw pain, myalgia, nausea, or vomiting) occurred that could not be tolerated by the subject, or if the subject experienced symptomatic hypotension, then the dose was reduced in a step-wise manner by 0.5 ng/kg/min every 30 minutes (±5 minutes) until a tolerated dose was determined or the infusion was stopped until the symptoms resolved at which point the study drug was reinitiated at a previously tolerated dose. If symptomatic hypotension or a dose-limiting adverse event occurred during administration of iloprost at the starting dose (i.e., 0.5 ng/kg/min), the infusion was reduced to 0.25 ng/kg/min. If the dose of 0.25 ng/kg/min was not tolerated due to symptomatic hypotension or if a dose-limiting adverse event occurred, the study drug was discontinued, and re-initiation of the infusion could have been attempted after the event had resolved or had been treated. Blood pressure and heart rate were monitored 15 minutes (±5 minutes) prior to and after all dose changes. The maximum tolerated dose was maintained for the remaining 6-hour daily period. At the end of the 6-hour infusion period, the dose was stopped. Subjects were to be monitored for up to 1 hour after completion of study drug infusion (i.e., vital signs were obtained 15 minutes [+5 minutes] and 1 hour [+15 minutes] after completion of the infusion).

On Days 2 to 5, the infusion was started using the highest infusion rate tolerated on the previous day without up- or down-titration, unless the subject did not tolerate the infusion or adverse events occurred that could not be tolerated by the subject and necessitated a reduction in the dose. Vital signs were measured prior to study drug administration and at 15 minutes (±5 minutes) prior to and after all dose changes during the infusion. Additionally, vital signs were monitored at 15 minutes (±5 minutes) and 1 hour (±15 minutes) after completion of the 6-hour infusion.

During the treatment period (Days 1 to 5), while subjects received study drug, the ePRO diary was not completed. No study assessments were performed on the 2 days following the end of treatment (Days 6 and 7 [i.e., Saturday and Sunday]) to allow the subject to rest and return to a schedule of normal daily living activity following the 5 days of infusions.

Subjects were contacted via telephone on Day 8 to ensure they resumed completion of the daily ePRO diary; subjects completed the ePRO diary from Day 8 through Day 21. On Day 22, subjects returned to the clinic for post-treatment evaluations. A follow-up visit occurred 30 days after the last administration of study drug (Day 35).

Subjects who discontinued study drug early remained in the study (unless the subject withdrew consent) and completed the daily ePRO diary from Day 8 to Day 21, including clinical laboratory assessments on the remaining missed infusion days as well as post-treatment study assessments.

The total duration of the study for a subject was up to approximately 9 weeks.

Number of subjects: Randomized-34 subjects; Completed-34 subjects

Diagnosis and Main Criteria for Inclusion: The population for this study included male and female subjects ≥18 years of age who met the following criteria: had a diagnosis of SSc, as defined by the 2013 American College of Rheumatology criteria/European League Against Rheumatism criteria; had a diagnosis or history of RP, self-reported or reported by a physician, with at least a 2-phase color change in figure(s) of pallor, cyanosis, and/or reactive hyperemia in response to cold exposure or emotion; and had a minimum of 10 symptomatic RP attacks, documented in the ePRO diary, that occurred over at least 3 separate days of the 5-day eligibility period.

Investigational Product and Comparator Information: Iloprost injection for IV use and matching placebo were supplied in vials packaged in a blinded study drug kit (10 vials per kit). The iloprost and placebo vials were identical, except 100 mcg of iloprost was added to the active study drug vials. The drug product was diluted with sodium chloride 0.9% in a drug reservoir (IV bag) prior to use.

### Criteria for Evaluation:

### Efficacy:

The primary efficacy parameter was the change in the weekly frequency of symptomatic RP attacks from baseline.

The exploratory efficacy parameters included changes from baseline to the end of the efficacy follow-up in the following:
Severity of RP attacks, as determined by the severity of RP attack symptoms (pain, numbness, tingling, and/or discomfort) (using a Numeric Rating Scale [NRS])
Raynaud's Condition Score
Hand function, as assessed by the Cochin Hand Functional Scale (CHFS)
Duration of symptomatic RP attacks
NRS for worst pain associated with symptomatic RP
Subject assessment of overall improvement in symptomatic RP (Patient Global Impression of Severity [PGI-S] and Patient Global Impression of Change [PGI-C])

### Pharmacokinetics:

The following PK parameters were calculated whenever possible, based on the plasma concentrations of iloprost:
Steady state plasma concentration just before infusion was stopped
Area under the plasma concentration versus time curve (AUC) from time of dosing to the last time point with measurable concentration (AUC₀₋ₗₐₛₜ)
AUC from time 0 extrapolated to infinity (AUC_{0-inf})
Percentage of AUC obtained by extrapolation (AUCₑₓₜᵣₐₚ%)
Terminal elimination constant (λz)
Plasma terminal elimination half-life (t_{1/2})
Plasma clearance (CL), calculated as dose/AUC_{0-inf}
Volume of distribution (V), calculated as dose/(λz×AUC_{0-inf})

### Safety:

Safety parameters included adverse events, physical examination findings, vital sign measurements (heart rate and blood pressure), 12-lead electrocardiogram (ECG) findings, and standard clinical laboratory measurements (chemistry and hematology).

### Efficacy and Safety Variables:

Symptomatic Raynaud's Phenomenon attacks: A symptomatic Raynaud's Phenomenon (RP) attack for this study was defined as at least 1 color change of the subject's finger(s) (blue, white, or red) associated with at least 1 symptom (pain, numbness, tingling, and/or discomfort of the finger[s]). The attack was considered over when the color changed back to pre-attack color (normal) and the symptoms returned to the subject's pre-attack level.

Electronic patient-reported outcomes diary: Subjects were provided with an ePRO diary at Visit 1 and trained on its use. Subjects were asked to complete the ePRO diary at the time points discussed above. Questionnaires allowed for documentation of frequency, severity, and duration of symptomatic RP attacks, as well as assessment of hand function. Specific questionnaires included the severity of RP attack symptoms (using a Numeric Rating Scale [NRS]). Raynaud's Condition Score. CHFS, and overall subject improvement (Patient Global Impression of Severity [PGI-S] and Patient Global Impression of Change [PGI-C]).

Severity of Raynaud's Phenomenon attack symptoms (using a Numeric Rating Scale): Raynaud's Phenomenon attacks are associated with significant discomfort (pain, numbness, tingling, and/or discomfort). Subjects were asked to rate the severity of RP attack symptoms (pain, numbness, tingling, and/or discomfort) using an 11-point NRS. Intensity was assessed as follows: 0 (no pain/numbness/tingling/discomfort). 1 to 3 (mild pain/numbness/tingling/discomfort). 4 to 6 (moderate pain/numbness/tingling/discomfort), and 7 to 10 (severe pain/numbness/tingling/discomfort).

Raynaud's Condition Score: The Raynaud's Condition Score asked subjects to rate their difficulty with RP condition on a given day from 0 (no difficulty) to 10 (extreme difficulty). Subjects were asked to consider the number of attacks they had on that day and how long each attack lasted. Subjects were asked to consider how much pain, numbness, or other symptoms the RP caused in their fingers (including painful sores) and how much the RP alone affected the use of their hands that day.

Cochin Hand Function Scale: Raynaud's Phenomenon attacks have a significant impact on hand function. The CHFS is an 18-item self-administered instrument that assesses hand function as it relates to daily activities. The CHFS has been validated for use in subjects with SSc.

Duration of symptomatic Raynaud's Phenomenon attacks: Subjects were asked to document the duration of each symptomatic RP attack within their ePRO diaries. For each attack, the subject recorded the duration in minutes.

Worst pain associated with symptomatic Raynaud's Phenomenon: Subjects were asked to rate the severity of the worst pain using an 11-point NRS within their ePRO diaries. Worst numbness, worst tingling, and worst discomfort were also assessed in the same way.

Overall subject improvement: On Day-1, subjects were asked to rate the overall severity of their symptomatic RP in the last week using a PGI-S score (0 to 10).

On Day 21, subjects were asked to rate the overall severity of their symptomatic RP in the last week using a PGI-S score (0 to 10) and the overall change in their symptomatic RP compared to the start of the study using a PGI-C score (much worse, a little worse, no change, a little better, or much better).

During the study, the Day-1 questionnaires were collected on the first day of the baseline ePRO diary completion period, and the end-of-study questionnaires were collected at Day 21 or 22.

### Summary of Results:

### Efficacy:

Overall, the number of subjects who completed at least 7 days of the ePRO diary for selected efficacy questions at baseline and the double-blind endpoint was high. During the 10- to 25-day baseline ePRO diary completion period, all 17 (100.0%) subjects in both the placebo group and the iloprost group completed at least 7 days of ePRO diary for all efficacy questions except for the question of “how many RP attacks in the past 24 hours” in the placebo group, where 16 (94.1%) subjects completed at least 7 days of ePRO diary. At the double-blind endpoint, 16 (94.1%) subjects in the placebo group and 17 (100.0%) subjects in the iloprost group completed at least 7 days of ePRO diary for all of the questions.

For the primary efficacy parameter, decreases in the weekly frequency of symptomatic RP attacks were observed from baseline to the double-blind endpoint for both the placebo group and the iloprost group, as analyzed by ANCOVA (least squares [LS] mean [standard error {SE} ] change of −14.32 [2.858] in the placebo group and −15.09 [2.961] in the iloprost group; p<0.0001) and nonparametric analysis (median [first quartile, third quartile] change of −11.67 [−18.86, −2.69] for the placebo group and −9.40 [−21.03, −7.50] for the iloprost group; statistical significance not assessed) (Table 1). The percent change in the weekly frequency of symptomatic RP attacks was also analyzed and showed directionally similar results to those seen with the weekly change parameter. However, the differences in the change in the weekly frequency of symptomatic RP attacks from baseline to the double-blind endpoint in the iloprost group compared to placebo were not statistically significant in any of the analytical measures listed above.

TABLE 1
Change in Weekly Frequency of Symptomatic
RP Attacks from Baseline

| Category | Placebo | Iloprost |
|---|---|---|
| Statistic | (N = 17) | (N = 17) |
| Number of subjects | | |
| n [1] | 17 | 17 |
| Baseline [2] | | |
| Mean (SD) | 42.32 (29.635) | 37.05 (18.643) |
| Double-blind endpoint [3] | | |
| Mean (SD) | 27.56 (25.416) | 23.57 (14.578) |
| Change from baseline | | |
| LS mean (SE) | −14.32 (2.858) | −15.09 (2.961) |
| 95% CI | (−20.15, −8.48) | (−21.14, −9.04) |
| p-value | <0.0001 | <0.0001 |
| Treatment comparison | | |
| of change vs placebo | | |
| LS mean difference | | −0.77 (4.047) |
| in change (SE) | | |
| 95% CI | | (−9.04, 7.49) |
| p-value | | 0.8498 |
| Shapiro-Wilk normality | | |
| test of residuals | | |
| p-value | | 0.0108 |

Note:
When a subject had no RP attacks in the past 24 hours, the frequency was considered as zero for that day. The LS means, SEs, CIs, and p-values came from an ANCOVA model with randomized treatment group and use of phosphodiesterase inhibitors at screening (yes, no) as factors and baseline as a covariate. For subjects in the Modified Intent-to-Treat Population with a missing primary efficacy endpoint, multiple imputation was used.
[1] n equaled the number of subjects with a result at both baseline and the double-blind endpoint.
[2] The baseline weekly frequency of symptomatic RP attacks was defined as the average number of weekly symptomatic RP attacks that occurred during the 10- to 25-day baseline ePRO diary completion period.
[3] The double-blind endpoint weekly frequency of symptomatic RP attacks was defined as the average number of weekly symptomatic RP attacks that occurred during Days 8 to 21, inclusive.
ANCOVA = analysis of covariance; CI = confidence interval; ePRO = electronic patient-reported outcomes; LS = least squares; RP = Raynaud's Phenomenon; SD = standard deviation; SE = standard error; vs = versus.

In a predefined analysis of seasonality, the LS mean (SE) difference in the change in the weekly frequency of symptomatic RP attacks from baseline to the double-blind endpoint in the iloprost group compared to placebo (treatment effect) was-4.87 (6.074) in the subgroup of subjects who were randomized prior to 3 Jun. 2019 and 2.04 (5.453) in the subgroup of subjects who were randomized after 3 Jun. 2019 (Table 2). These results were numerically consistent with an effect of season; however, these comparisons were not statistically significant (p=0.4292 and p=0.7116 for the comparisons between iloprost and placebo in the subgroups of subjects randomized prior to and after 3 Jun. 2019, respectively). When comparing the treatment difference in subgroups of subjects who were randomized prior to and after 3 Jun. 2019, the LS mean (SE) difference was-6.91 (8.121) for the change in weekly frequency of symptomatic RP attacks from baseline to the double-blind endpoint; however, this observation was not statistically significant (p=0.4021). Seasonal variation has been observed in previous reports. Wigley, F. M. et al. Ann Intern Med. 1994, 120, 199-206; Wigley, F. M. et al. J Rheumatol. 1992, 19, 1407-1414.

TABLE 2
Change in Weekly Frequency of Symptomatic RP Attacks from Baseline

| | Randomization Date | | Randomization Date | |
|---|---|---|---|---|
| | Prior to 3 Jun. 2019 | | After 3 Jun. 2019 | |
| Category | Placebo | Iloprost | Placebo | Iloprost |
| Statistic | (N = 7) | (N) | (N = 10) | (N = 9) |
| Number of subjects | | | | |
| n [1] | 7 | 8 | 10 | 9 |
| Baseline [2] | | | | |
| Mean (SD) | 41.22 (26.777) | 36.85 (19.235) | 43.10 (32.889) | 37.23 (19.269) |
| Double-blind endpoint [3] | | | | |
| Mean (SD) | 31.10 (32.602) | 23.39 (11.923) | 25.09 (20.573) | 23.73 (17.337) |
| Change from baseline | | | | |
| LS mean (SE) | −9.95 (4.438) | −14.83 (4.180) | −17.41 (3.739) | −15.38 (4.082) |
| Treatment comparison | | | | |
| of change vs placebo | | | | |
| LS mean difference in | | −4.87 (6.074) | | 2.04 (5.453) |
| change (SE) | | | | |
| 95% CI | | (−17.32, 7.57) | | (−9.13, 13.21) |
| p-value | | 0.4292 | | 0.7116 |
| Subgroup comparison | | | | |
| of prior vs after | | | | |
| LS mean difference | | | | −6.91 (8.121) |
| in change (SE) | | | | |
| 95% CI | | | | (−23.54, 9.73) |
| p-value | | | | 0.4021 |

[1]-[3]—see Table 1.

Statistically significant decreases (or improvements in the case of PGI-C) from baseline to the double-blind endpoint were observed within treatment group for both the placebo group and the iloprost group in most of the exploratory endpoints analyzed in this study (listed below). However, the differences in the iloprost group compared to placebo were not statistically significant:
The change in average overall severity of RP attack symptoms (Table 3)
The change in average Raynaud's Condition Score
The change in average weekly duration of symptomatic RP attacks (minutes) (Table 4)
The change in average worst symptoms (pain, numbness, tingling, and discomfort) associated with symptomatic RP
Subject assessment of overall improvement using PGI-S and PGI-C

TABLE 3
Change in Average Overall Severity of
Symptomatic RP Attacks from Baseline

| Category | Placebo | Iloprost |
|---|---|---|
| Statistic | (N = 17) | (N = 17) |
| Number of subjects | | |
| n [1] | 17 | 17 |
| Baseline [2] | | |
| Mean (SD) | 6.12 (2.245) | 5.30 (1.818) |
| Double-blind | | |
| endpoint [3] | | |
| Mean (SD) | 4.44 (2.473) | 3.39 (2.169) |
| Change from baseline | | |
| LS mean (SE) | −1.65 (0.424) | −2.12 (0.439) |
| 95% CI | (−2.51, −0.78) | (−3.01, −1.22) |
| p-value | 0.0005 | <0.0001 |
| Treatment comparison | | |
| of change vs placebo | | |
| LS mean difference | | −0.47 (0.604) |
| in change (SE) | | |
| 95% CI | | (−1.71, 0.76) |
| p-value | | 0.4409 |

[1]-[3]—see Table 1.

TABLE 4
Change in Average Weekly Duration of Symptomatic
RP Attacks from Baseline (minutes)

| Category | Placebo | Ilopcost |
|---|---|---|
| Statistic | (N = 17) | (N = 17) |
| Number of subjects | | |
| n [1] | 17 | 17 |
| Baseline [2] | | |
| Mean (SD) | 822.53 (630.482) | 586.97 (584.577) |
| Double-blind | | |
| endpoint [3] | | |
| Mean (SD) | 608.09 (654.620) | 345.82 (402.713) |
| Change from baseline | | |
| LS mean (SE) | −183.60 (75.519) | −267.49 (78.273) |
| 95% CI | (−337.83, −29.37) | (−427.35, −107.64) |
| p-value | 0.0212 | 0.0018 |
| Treatment comparison | | |
| of change vs placebo | | |
| LS mean difference | | −83.89 (107.728) |
| in change (SE) | | |
| 95% CI | | (−303.90, 136.12) |
| p-value | | 0.4422 |

[1]-[3]—see Table 1.

### Pharmacokinetics:

Table 5 shows the summary of PK parameters for the PK population. In general, the geometric mean of steady state plasma concentration (Css), AUC₀₋ₗₐₛₜ, and AUC_{0-inf} of iloprost increased as dose increased across the 4 maximum tolerated dose levels (from 0.50 to 2.00 ng/kg/min), with the exception of Css and AUC₀₋ₗₐₛₜ at the 1.50 ng/kg/min dose level.

The mean t½ of iloprost ranged from 0.381 to 0.471 hours across the 4 dose levels.

TABLE 5
Summary of Pharmacokinetic Parameters

| | Maximum | Maximum | Maximum | Maximum |
|---|---|---|---|---|
| | Tolerated Dose | Tolerated Dose | Tolerated Dose | Tolerated Dose |
| Parameter | Level | Level | Level | Level |
| Statistic | 0.50 ng/kg/min | 1.00 ng/kg/min | 1.50 ng/kg/min | 2.00 ng/kg/min |
| Cₛₛ (pg/mL) | | | | |
| n | 1 | 2 | 2 | 4 |
| Geometric mean | 19.60 (NA) | 51.31 (20.7) | 32.90 (284.8) | 76.82 (56.9) |
| (geometric CV %) | | | | |
| t_{1/2} (h) | | | | |
| n | | 1 | 1 | 1 |
| Mean (SD) | | 0.41 (NA) | 0.450 (NA) | 0.381 (NA) |
| AUC_{0-inf} | | | | |
| (h*pg/mL) | | | | |
| n | | 1 | 1 | 1 |
| Geometric mean | | 378.9 (NA) | 602.7 (NA) | 700.6 (NA) |
| (geometric CV %) | | | | |
| AUC₀₋ₗₐₛₜ | | | | |
| (h*pg/mL) | | | | |
| n | 1 | 2 | 2 | 4 |
| Geometric mean | 116.0 (NA) | 314.7 (23.9) | 202.7 (303.2) | 478.0 (55.6) |
| (geometric CV %) | | | | |
| CLₛₛ (L/h) | | | | |
| N | | 1 | 1 | 1 |
| Mean (SD) | | 91.22 (NA) | 6.01 (NA) | 64.53 (NA) |
| Vₛₛ (L) | | | | |
| n | | 1 | 1 | 1 |
| Mean (SD) | | 62.01 (NA) | 49.34 (NA) | 35.49 (NA) |

Note:
Geometric CV % = 100 × (exp(SD²)₋₁)^{0.5}, where SD was the SD of the logarithmic-transformed data. One subject was excluded from analysis because the 6-hour postdose PK sample was collected 2 minutes after the infusion stopped.
AUC_{0-inf} = area under the plasma concentration versus time curve from time 0 extrapolated to infinity; AUC0-last = area under the plasma concentration versus time curve from time of dosing to the last time point with measurable concentration; CLss = plasma clearance at steady state; Css = steady state plasma concentration; CV % = percent coefficient of variation; max = maximum; min = minimum; NA = not applicable; PK = pharmacokinetic(s); SD = standard deviation; t½ = plasma terminal elimination half-life; Vss = volume of distribution at steady state.

### Safety:

No subjects experienced a serious adverse event (SAE), adverse event of special interest (AESI), treatment-emergent adverse event (TEAE) leading to discontinuation of study drug, or TEAE leading to death in this study.

Overall, 31 (91.2%) subjects experienced a TEAE: 14 (82.4%) subjects in the placebo group and 17 (100.0%) subjects in the iloprost group. The majority of TEAEs were considered mild to moderate in severity. Two (11.8%) subjects in the iloprost group experienced TEAEs that were considered severe by the Investigator.

The most commonly reported TEAEs (based on the total number of subjects who experienced this TEAE) were headache (21 [61.8%] subjects total: 5 [29.4%] subjects in the placebo group and 16 [94.1%] subjects in the iloprost group), nausea (14 [41.2%] subjects total: 3 [17.6%] subjects in the placebo group and 11 [64.7%] subjects in the iloprost group), abdominal pain (5 [14.7%] subjects total: 1 [5.9%] subject in the placebo group and 4 [23.5%] subjects in the iloprost group), and flushing (5 [14.7%] subjects total: 0 [0.0%] subjects in the placebo group and 5 [29.4%] subjects in the iloprost group).

There were no clinically significant changes in chemistry or hematology laboratory values during the study.

There were no TEAEs related to vital signs, physical examination findings, or ECGs in this study.

There were no hypertensive or hypotensive events reported in this study. There was no bradycardia or tachycardia reported in this study.

### Conclusions

This pilot, multicenter, double-blind, randomized, placebo-controlled study confirmed the ability to evaluate the effect of iloprost on the symptomatic relief of symptomatic RP attacks using an ePRO diary in subjects with SSc. The results also confirmed that the iloprost dosing titration algorithm could be safely used in subjects with SSc. All adverse events related to the study drug as assessed by the Investigator were expected and consistent with the known safety profile of iloprost. There were no deaths, SAEs, AESIs, or TEAEs leading to study drug discontinuation during this study.

The study was not adequately powered to test the efficacy of iloprost for reducing the frequency of RP attacks compared to placebo. The seasonality finding is consistent with previous iloprost studies conducted in the spring and summer months, and the study was underpowered.

### Example 2: Iloprost Phospholipid Formulation

Iloprost is formulated as a phospholipid depot formulation containing 0.02% iloprost (200 mcg/mL; 0.5 mL primary packaging). The remainder of the formulation is as listed in Table 6.

TABLE 6
Iloprost phospholipid formulation

| | Amount | |
|---|---|---|
| Ingredient | (% by weight) | Function |
| Iloprost | 0.02% | Drug |
| ethanol 200 Proof, USP | 0.081% | Solubilizer |
| Tromethamine USP | 0.024% | Buffer |
| Sodium Hydroxide | QS | Adjust pH to 8.3 (or in |
| | | the range of 8.0-8.9) |
| Lectin | 15-50% | Phospholipid component |
| Oil | 15-50% | Phospholipid component |
| Water | up to 70% | Phospholipid component |

### Example 3. Iloprost Pharmacokinetic Model

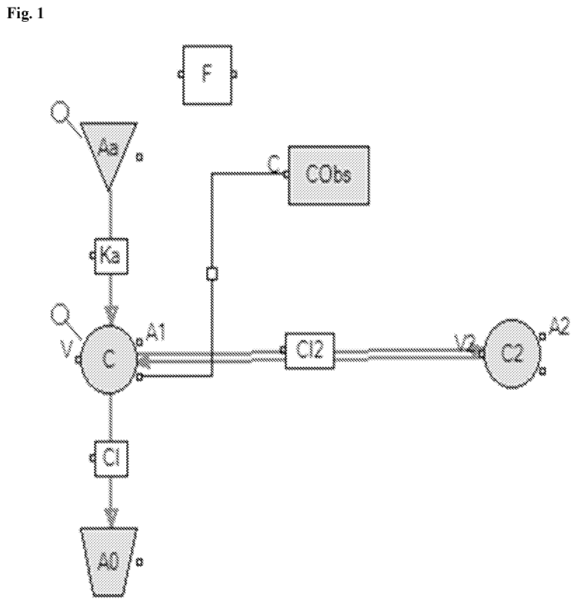

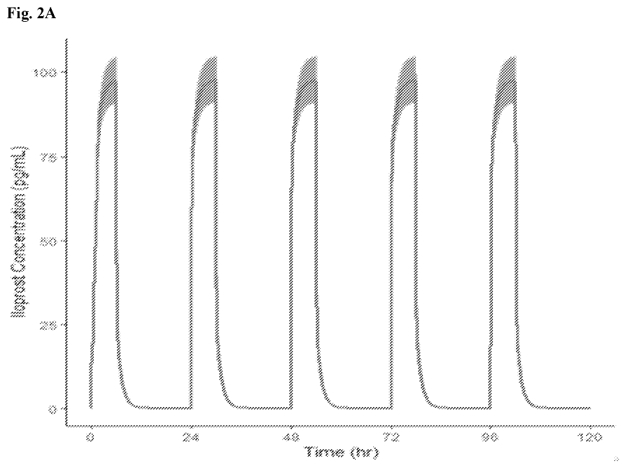

A PK model for iloprost based on published data in the literature was developed. Conclusions from the structural model building suggested that 2-compartment disposition model with first-order absorption and linear clearance provided the best fit for IV infusion and inhaled digitized data. In addition, the proportional error model was selected as the final residual error model. The schematic of iloprost 2-compartment PK model is shown in FIG. 1.

In FIG. 1, Aa is an amount administered to the absorption compartment, Ka is a first-order absorption rate constant, A1 is an amount of iloprost in compartment 1, F is bioavailability for inhaled iloprost, Cobs is the observed concentration, C is the concentration in compartment 1, C1 is the clearance from compartment 1, V is the volume of distribution from compartment 1, A0 is the amount excreted from central compartment, C12 is the intercompartmental clearance, V2 is the volume of distribution from compartment 2, C2 is the concentration in compartment 2, and A2 is the amount of iloprost in compartment 2.

The iloprost model-derived PK parameters incorporating variability estimates are shown Table 7. The PK model derived clearance from central compartment (C1=20.5 ml/min/kg, Table 7) was comparable to the literature reported non-compartmental clearance (C1=21 ml/min/kg at 1 ng/kg/min for 45 minutes; C1=20.6 ml/min/kg at 3 ng/kg/min for 45 minutes; C1=24 ml/min/kg at 2 ng/kg/min for 4 hours. The final iloprost structural PK model provided reasonable estimates for iloprost PK parameters and could be used for exploratory evaluation. In addition, the PK model derived parameters showed iloprost bioavailability of 88% when it is administered as an inhaled dosing as compared to the IV infusion using the digitized data (Table 7).

TABLE 7
Iloprost 2-Compartment PK Model Derived PK Parameters

| | | | Standard | |
|---|---|---|---|---|
| Parameter | Estimate | Units | Error | CV % |
| V | 190 | ml/kg | 36.7 | 19 |
| Cl | 20.5 | ml/min/kg | 1.02 | 5 |
| Ka | 0.231 | 1/min | 0.034 | 15 |
| V2 | 321 | ml/kg | 65 | 20 |
| Cl2 | 6.23 | ml/min/kg | 1.254 | 20 |
| F | 88.1 | % | 0.06 | 7.1 |

Cl = the clearance from central compartment;
V = volume of distribution from central compartment;
Cl2 = intercompartmental clearance;
V2 = volume of distribution from peripheral compartment;
Ka = absorption constant;
F = bioavailability when it is administered as an inhaled dosing compared to IV infusion using digitized data

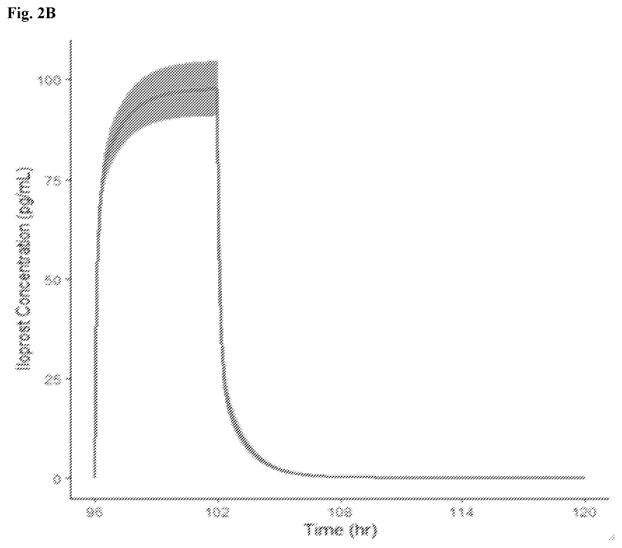

The mean PK profile of iloprost was simulated based on dosing scenarios in Table 8. FIGS. 2A-2B shows the mean simulated PK profile of iloprost from a 2.0 ng/kg/min over 6 hours daily infusion over 5 days showing no accumulation of iloprost (FIG. 2A) and at Day 5 (FIG. 2B). The lines in the graphs of FIGS. 2A-2B are the means of all simulations and the shading around the peaks is the 95% confidence interval for all simulations. The simulated iloprost PK exposure after daily IV infusion at 2.0 ng/kg/min over 6 hours over 5 days (scenario A) are shown in Table 9. The AUCs are 24 hour AUCs after a single day iloprost dosing, respectively.

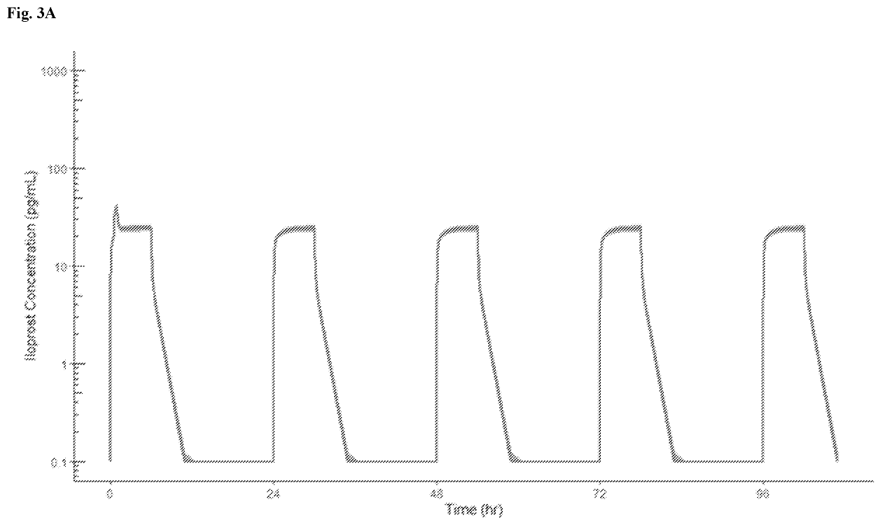

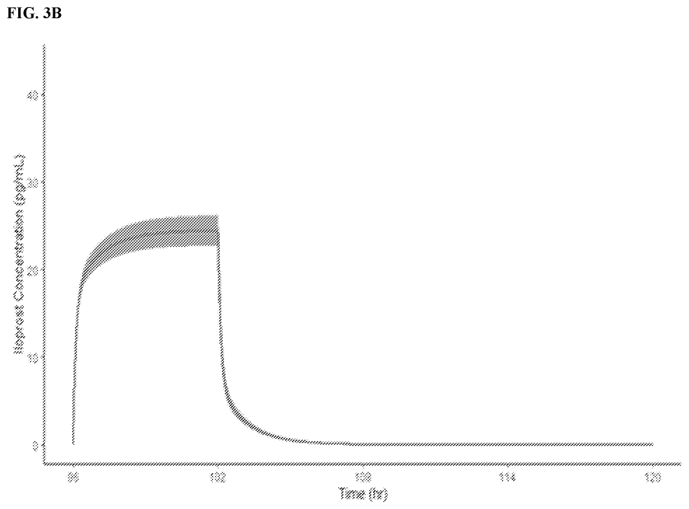

TABLE 8
Dosing Scenarios for PK Simulation

| Dosing | Dosing Schedule | |
|---|---|---|
| Scenarios | Day 1 | Day 2 to Day 5 |
| IV infusion dosing | 0.5 ng/kg/min for 30 minutes, | 2 ng/kg/min for |
| scenario A | 1 ng/kg/min for next 30 minutes, | 360 minutes |
| (FIGS. 2A-2B) | 1.5 ng/kg/min for next 30 minutes, | every 24 hours |
| | 2 ng/kg/min for next 270 minutes | |
| IV infusion dosing | 0.5 ng/kg/min for 30 minutes, | 0.5 ng/kg/min |
| scenario B | 1 ng/kg/min for next 30 minutes, | for 360 minutes |
| (FIGS. 3A-3B) | 0.5 ng/kg/min for next 300 minutes | every 24 hours |

TABLE 9
Simulated Iloprost PK Exposure After
IV Infusion at 2.0 ng/kg/min over 6 h

| Iloprost PK | | 5^{th} | 95^{th} |
|---|---|---|---|
| Parameter | Mean | Percentile | Percentile |
| AUC (pg*hr/mL) | 586 | 547 | 630 |
| Cmax (pg/mL) | 97 | 91 | 105 |

FIGS. 3A-3B shows the mean simulated PK profile of iloprost from a 0.5 ng/kg/min over 6 hours daily infusion over 5 days showing no accumulation of iloprost (FIG. 3A) and at Day 5 (FIG. 3B). The lines in the graphs of FIGS. 3A-3B are the means of all simulations and the shading around the peaks is the 95% confidence interval for all simulations. The simulated iloprost PK exposure after daily IV infusion at 0.5 ng/kg/min over 6 hours over 5 days (scenario B, Table 8) are shown in Table 10. The AUCs are 24 hour AUCs after a single day iloprost dosing, respectively.

TABLE 10
Simulated Iloprost PK Exposure After
IV Infusion at 0.5 ng/kg/min over 6 h

| Iloprost PK | | 5^{th} | 95^{th} |
|---|---|---|---|
| Parameter | Mean | Percentile | Percentile |
| AUC (pg*hr/mL) | 147 | 137 | 157 |
| Cmax (pg/mL) | 24 | 23 | 26 |

Based on the simulated PK profiles, Cmax and AUC for the controlled release iloprost formulation were estimated for subcutaneous or intramuscular injection as well as for continuous IV infusion as shown in Table 11.

TABLE 11
Estimated Cmax and AUC for controlled
release iloprost formulation

| | | | | Day 1 to 5 |
|---|---|---|---|---|
| | | Daily | | Cumulative |
| Regimen | | Cₘₐₓ | Daily AUC | AUC |
| Description | Dose | (pg/mL) | (pg*hr/mL) | (pg*hr/mL) |
| IV Infusion | 2.0 ng/kg/min | 97 | 513 (Day 1) | 2857 |
| Iloprost | over 6 hours | | 586 (Day 2-5) | |
| Controlled | 716 ng/kg* | 97 | 586 (Day 1-5) | 2930 |
| Release System | | | | |
| IV Infusion | 0.5 ng/kg/min | 24 | 159 (Day 1) | 747 |
| Iloprost | over 6 hours | | 147 (Day 2-5) | |
| Controlled | 177 ng/kg* | 24 | 147 (Day 1-5) | 735 |
| Release System | | | | |

*Dose is calculated by controlled release constant multiplied by the duration (6 hours). The controlled release constant (ng/kg/min) is calculated by iloprost total clearance (20.5 mL/min/kg) multiplied by the target steady state plasma concentration (24 pg/mL = for 0.5 ng/kg/min infusion and 97 pg/mL = 2.0 ng/kg/min infusion rates).

The published data in the literature used for simulation in this example includes: Krause, W., and Krais, T. (1986) Eur. J Clin. Pharmacol 30, 61-68; Krause, W., and Krais, T. (1987) Eur. J Clin. Pharmacol 32, 597-605; and Olschewski, H., Rohde, B., Behr, J., Ewert, R., Gessler, T., Ghofrani, H. A., and Schmehl, T. (2003) Chest 124, 1294-1304.

### Example 4. Light and Heat Exposure Study

Two iloprost formulations with different pHs (Table 12) were evaluated for light exposure and heat exposure. Approximately 100 mg of iloprost in a vial was dissolved in approximately 2 mL of ethanol under anaerobic conditions (glove bag). The vial was capped shaken by hand until iloprost dissolved. Total of 3 vials of iloprost was dissolved in ethanol. The content of the three vials were quantitatively transferred to a 50 mL vial and the vial was rinsed with 2 mL×3 ethanol. The 50 mL vial contained approximately 300 mg iloprost dissolved in approximately 30.9 mL of ethanol (96%). The iloprost/ethanol solution was quantitatively transferred into a 5 L media bottle containing tromethamine buffer solution which was sparged with nitrogen for approximately 24 minutes. (27.00 g NaCl and 0.726 g tromethamine in approximately 2100 g water for injection (WFI); pH 9.61). The 50 mL vial which contained the iloprost/ethanol solution was rinsed three times with a total of 90 mL WFI (pre-sparged with N2). Then the pH of the solution was adjusted with 1 N HCl as indicated in Table 12. Sufficient amount of WFI (pre-sparged with N2) was added to provide a final solution weight of 3.013 kg, mixed, and the solution was blanketed with nitrogen. The density, osmolality, and pH of each formulation was determined. Also, both formulations were assayed by HPLC for iloprost and total impurities.

Approximately 200 mL of the prepared formulation was filtered through a Millipak 200, 0.22μ filter for light exposure study and heat exposure study (100 mL for each study).

TABLE 12
Iloprost Samples

| | | | | | Iloprost | |
|---|---|---|---|---|---|---|
| | | Density | Osmolality | | Assay | % Total |
| Sample ID | Appearance | (g/L) | (mOsm/kg) | pH | (mg/mL, %) | Impurities |
| Formulation A | Clear, | 1.0044 | 305 | 7.3 | 0.115, 114.8 | 2.76 |
| | colorless | | | | | |
| | solution | | | | | |
| Formulation B | Clear, | 1.0034 | 418 | 8.1 | 0.109, 109.0 | 1.69 |
| | colorless | | | | | |
| | solution | | | | | |

Light Exposure Study: Approximately 1.1 mL of formulation was placed each into 60 clean 2 cc vials and the vials were capped with 13 mm caps. Three sets of ten vials were wrapped with aluminum foil (A1) and three sets of ten vials were placed in a clear bag (A2). The samples were placed under ambient light. Ten samples were pulled at the time indicated in Table 13 and analyzed for pH and assayed by HPLC for iloprost and impurities.

TABLE 13
Light Exposure Study

| | | | | | Iloprost | Total |
|---|---|---|---|---|---|---|
| | | Test | | | Assay | Impurities |
| Time | | Condition | Formulation | pH | (mg/mL, %) | (%) |
| 0 | hr | A1 | A | 7.0 | 0.114, 114.2 | 2.98 |
| 24 | hr | A1 | A | 7.0 | 0.115, 114.7 | 3.41 |
| 48 | hr | A1 | A | 7.0 | 0.114, 114.3 | 3.53 |
| 0 | hr | A2 | A | 7.0 | 0.114, 114.2 | 2.98 |
| 24 | hr | A2 | A | 7.0 | 0.114, 114.3 | 3.44 |
| 48 | hr | A2 | A | 7.0 | 0.114, 114.4 | 3.45 |
| 0 | hr | A1 | B | 8.1 | 0.108, 108.0 | 1.74 |
| 24 | hr | A1 | B | 8.0 | 0.108, 107.9 | 1.73 |
| 47 hr 35 min | | A1 | B | 8.0 | 0.108, 107.7 | 1.81 |
| 0 | hr | A2 | B | 8.0 | 0.108, 107.7 | 1.82 |
| 24 | hr | A2 | B | 8.0 | 0.108, 107.7 | 1.75 |

| | | | | | |
|---|---|---|---|---|---|
| 47 hr 35 min | A2 | B | 8.0 | 0.108, 107.9 | 1.77 |

Heat Exposure Study: Approximately 1.1 mL of formulation was placed each into 40 clean 2 cc vials and the vials were capped with 13 mm caps. One set of 20 vials were wrapped with autoclave tape, placed into an autoclave bag and labeled as B1. Another set of 20 vials were wrapped with autoclave tape, placed into an autoclave bag and labeled as B2. All vials were exposed through one sterilization cycle at 121° C. for approximately 30 minutes, then B1 vials were analyzed for pH and assayed by HPLC. The B2 vials were then exposed to a second sterilization cycle at ≥121° C. for ≥15 minutes. The B2 vials were analyzed for pH and assayed by HPLC for iloprost and impurities. See Table 15.

TABLE 14
Heat Exposure Study

| Test | | | Iloprost Assay | |
|---|---|---|---|---|
| Condition | Formulation | pH | (mg/mL, %) | Total Impurities (%) |
| B1 | A | 7.1 | 0.109, 109.2 | 4.76 |
| B2 | A | 7.2 | 0.107, 106.7 | 6.46 |
| B1 | B | 8.0 | 0.108, 107.5 | 1.58 |
| B2 | B | 8.0 | 0.106, 106.2 | 2.19 |

In both studies, Formulation B with the higher pH (pH 8.0-8.1) resulted in less total impurities, indicating that iloprost is more stable at higher pH of about 8.0-8.1 than at pH of about 7.0-7.2.

### Example 5. Pharmacokinetic Study

The pharmacokinetics of three iloprost long-acting phospholipid gel (PG) formulations (Formulations 1, 2, and 3) in rats were determined following a single subcutaneous administration to Sprague-Dawley rats. A total of 18 male Sprague-Dawley rats were assigned to study (target weight of each rat was at least 300-400 g). The animals were not fasted prior to dosing.

Each animal in Groups 1 to 3 received a single subcutaneous (SC) dose of Formulation 1, 2, or 3, respectively, as shown in Table 15. Unless otherwise indicated, subcutaneous doses were administered via bolus injection between the skin and underlying layers of tissue in the scapular region on the back of each animal. The hair was be clipped from the injection site prior to dosing.

TABLE 15
Dosing Information

| | | | | Dose | |
|---|---|---|---|---|---|
| | | Dose | Dose | Concen- | No. of |
| | | Level | Volume | tration | Male |
| Group | Treatment | (μg/animal)^{a} | (mL) | (μg/mL) | Animals |
| 1 | Formulation 1 | 21.4 | 0.1 | 214 | 6 |
| 2 | Formulation 2 | 19.2 | | 192 | 6 |
| 3 | Formulation 3 | 19.3 | | 193 | 6 |

^{a}Approximately 50 to 60 μg/kg dose of iloprost, assuming a 300 g rat

1 mL blood (plasma) samples were collected on Day 1 at alternating time points from 2 cohorts of 3 animals/treated group at approximately 0.25, 1, 2, 4, 8, 12, 24, 48, and 72 hours postdose from a sublingual vein. Anticoagulant used was K2EDTA. All blood samples were placed on wet ice following collection until centrifuged. All resultant plasma was placed in a single aliquot and any clotted samples were noted. Final sample was stored frozen (−60 to −90° C.). Plasma samples were analyzed for concentration of iloprost.

The mean, standard deviation (SD), and coefficient of variation (CV) were calculated for iloprost plasma concentrations at each time point. Concentrations less than the lower limit of quantitation (LLOQ=20 pg/mL) were set to 0 for pharmacokinetic analysis. A composite plasma concentration-time profile was constructed for each respective dose group from which pharmacokinetic parameters were derived.

The mean iloprost plasma concentration-time profiles from iloprost long-acting PG formulation-treated animals were analyzed using model-independent methods (see Gibaldi M, Perrier D. Pharmacokinetics. 2nd ed. New York: Marcel Dekker, Inc., 1982:409-417). For each dose group, the following pharmacokinetic parameters were determined: maximum observed plasma concentration (Cmax), time of maximum observed plasma concentration (Tₘₐₓ), and area under the plasma concentration-time curve (AUC). The AUC from time 0 to 72 hours (AUC₀₋₇₂ hr) and the AUC from time 0 to infinity (AUC_{INF}) were calculated by the linear trapezoidal method for all dose groups with at least 3 consecutive quantifiable concentrations. For Day 1, 0 was used as an estimate of the 0-hour concentration.

The percent of AUC extrapolated (% AUCExtrap) for AUC_{INF} was calculated as:

$$\% AUCExtrap = [ ( AUCINF - AUCTlast ) / AUCINF ] \times 100$$

All AUC_{INF} values were calculated with less than 25% extrapolation.

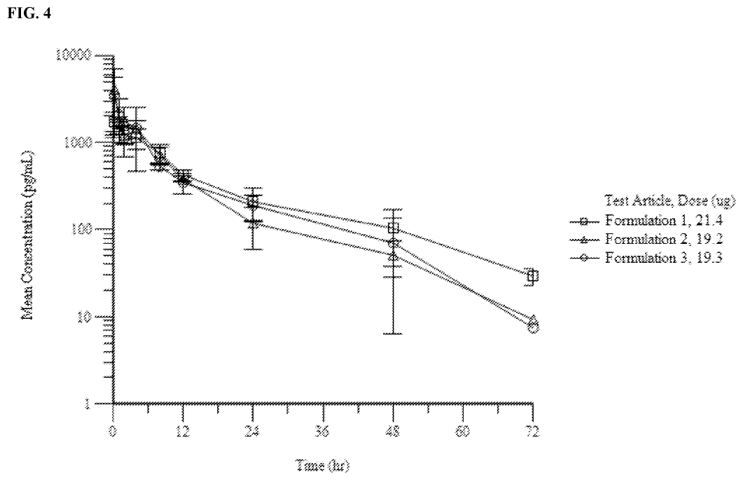

Iloprost plasma pharmacokinetic parameters are provided in Table 16. Mean iloprost plasma concentration-time profiles are illustrated in FIG. 4. Mean iloprost plasma concentration data are provided in Table 17.

TABLE 16
Iloprost Pharmacokinetic Parameters Following a Single SC Injection

| | Cₘₐₓ | Cₘₐₓ/Dose | Tₘₐₓ | Tₗₐₛₜ | AUC_{0-72 hr} | AUC_{0-72 hr}/Dose | AUC_{INF} | AUC_{INF}/Dose |
|---|---|---|---|---|---|---|---|---|
| Group | (pg/mL) | (pg/mL/ug) | (hr) | (hr) | (hr*pg/mL) | (hr*pg/mL/ug) | (hr*pg/mL) | (hr*pg/mL/ug) |
| 1 | 1740 | 81.5 | 0.25 | 72 | 20500 | 958 | 21200 | 993 |
| 2 | 4080 | 213 | 0.25 | 72 | 19900 | 1040 | 20100 | 1050 |
| 3 | 3450 | 179 | 0.25 | 72 | 19800 | 1020 | 19900 | 1030 |

TABLE 17
Iloprost Pharmacokinetic Parameters
Following a Single SC Injection

| | Dose | Time | | Mean | | |
|---|---|---|---|---|---|---|
| Group | (ug) | (hr) | N | (pg/mL) | SD | CV % |
| 1 | 21.4 | 0.25 | 3 | 1740 | 513 | 29.4 |
| | | 1 | 3 | 1600 | 335 | 20.9 |
| | | 2 | 3 | 1220 | 236 | 19.4 |
| | | 4 | 3 | 1130 | 295 | 26.1 |
| | | 8 | 3 | 751 | 178 | 23.8 |
| | | 12 | 3 | 426 | 59.4 | 13.9 |
| | | 24 | 3 | 209 | 86.2 | 41.3 |
| | | 48 | 3 | 104 | 65.7 | 63.3 |
| | | 72 | 3 | 29.3 | 6.62 | 22.6 |
| 2 | 19.2 | 0.25 | 3 | 4080 | 2950 | 72.4 |
| | | 1 | 3 | 1780 | 36.1 | 2.03 |
| | | 2 | 3 | 1730 | 792 | 45.7 |
| | | 4 | 3 | 1450 | 352 | 24.4 |
| | | 8 | 3 | 721 | 164 | 22.8 |
| | | 12 | 3 | 380 | 26.9 | 7.07 |
| | | 24 | 3 | 119 | 59.5 | 50.2 |
| | | 48 | 3 | 51.0 | 22.8 | 44.8 |
| | | 72 | 3 | 9.30 | 16.1 | 173 |
| 3 | 19.3 | 0.25 | 3 | 3450 | 2130 | 61.7 |
| | | 1 | 3 | 2100 | 1090 | 51.6 |
| | | 2 | 3 | 1120 | 440 | 39.4 |
| | | 4 | 3 | 1470 | 1010 | 68.7 |
| | | 8 | 3 | 532 | 43.1 | 8.10 |
| | | 12 | 3 | 347 | 87.5 | 25.2 |
| | | 24 | 3 | 190 | 60.7 | 32.0 |
| | | 48 | 3 | 70.4 | 64.0 | 90.9 |
| | | 72 | 3 | 7.50 | 13.0 | 173 |

The variability in mean plasma concentrations, as measured by CV values, was similar between dose groups and ranged from 2.03% to 173% on Day 1. The higher variability observed was the result of converting below the quantification limit results to zero and averaging with quantifiable results. The range with those values excluded was from 2.03% to 72.4%. Iloprost was quantifiable up to 72 hours postdose for Formulations 1, 2, and 3 following a single SC injection. Tₘₐₓ values were observed at 0.25 hours postdose for Formulations 1, 2, and 3.

Following a single SC injection of iloprost long-acting formulations, dose normalized Cmax values were lower for Formulation 1 and similar between Formulations 2 and 3, whereas dose normalized AUC_{0-72 hr} and AUC_{INF} values were generally similar between Formulations 1, 2, and 3.

The disclosures of all publications, patents, patent applications and published patent applications referred to herein by an identifying citation are hereby incorporated herein by reference in their entirety.

In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A sustained release pharmaceutical composition for injection, comprising iloprost or a pharmaceutically acceptable salt or stereoisomer thereof and a pharmaceutically acceptable carrier or excipient:
wherein the composition releases iloprost over a period of about 5 days to about 7 days.

2. The pharmaceutical composition of claim 1, wherein the composition provides a serum concentration of iloprost at steady state (Css) in the range of about 10 pg/mL to about 50 pg/mL.

3. The pharmaceutical composition of claim 1, wherein the composition provides a serum concentration of iloprost at steady state (Css) in the range of about 20 pg/mL to about 40 pg/mL.

4. The pharmaceutical composition of any one of claims 1-3, wherein a single dose of the composition comprises about 5.0 mcg iloprost or a pharmaceutically acceptable salt or stereoisomer thereof per weight of the subject (kg) to about 10.0 mcg/kg.

5. The pharmaceutical composition of any one of claims 1-4, wherein the injection is a subcutaneous injection.

6. The pharmaceutical composition of any one of claims 1-5, wherein a single daily dose comprises iloprost or a pharmaceutically acceptable salt or stereoisomer thereof in about 0.01% to about 20% by weight of the composition.

7. The pharmaceutical composition of any one of claims 1-6, wherein the composition comprises a phospholipid.

8. The pharmaceutical composition of claim 7, wherein the phospholipid is lecithin, phosphatidylcholine, phosphotidylethanolamine, phosphotidylserine, phosphatidylinositol, phosphoglyceride, phosphoglycerol, phospholipid, sphingosine, ganglioside, phytosphingosine, diacylglycerol, phosphocholine, phosphoethanolamine, hosphotidylserine, lysophospholipid, pegylated phospholipid, mixed chain phospholipid, or a combinations thereof.

9. The pharmaceutical composition of claim 8, wherein the lecithin is soy lecithin, egg lecithin, or a combination thereof.

10. The pharmaceutical composition of any one of claims 7-9, wherein the composition comprises a phospholipid in about 20% to about 80% by weight of the composition.

11. The pharmaceutical composition of any one of claims 1-10, wherein the composition comprises oil.

12. The pharmaceutical composition of claim 11, wherein the oil is synthetic oil, a vegetable oil, sesame oil, a medium chain oil, silicone oil, ethyl oleate, fatty acid, vitamin E, vitamin E succinate, cholesterol, triglyceride oil, or a mixture thereof.

13. The pharmaceutical composition of any one of claims 1-12, wherein the pharmaceutically acceptable excipient is sucrose, dextrose, lactose, glucose, trehalose, maltose, mannitol, sorbitol, glycerol, amylose, starch, amylopectin, triglycerides, fatty acids, carbohydrates, or a mixture thereof.

14. The pharmaceutical composition of any one of claims 1-13, wherein the pharmaceutically acceptable excipient is an acidifying agent, an alkalizing agent, a pH buffering agent, a metal ion chelator, an antioxidant, a preservative, a tonicity/osmotic pressure modifier, a condensing agent, a solubilizing agent, or a mixture thereof

15. The pharmaceutical composition of any one of claims 1-13, wherein the pharmaceutically acceptable carrier is ethanol, propylene glycol, glycerol, sorbitol, polyethylene glycol, silicone oil, glycofurol, ethyl oleate, or a mixture thereof.

16. The pharmaceutical composition of any one of claims 1-15, wherein the composition is an injectable gel composition.

17. The pharmaceutical composition of any one of claims 1-16, wherein the composition is a nanoemulsion.

18. The pharmaceutical composition of any one of claims 1-5, wherein the composition comprises a peptide.

19. The pharmaceutical composition of claim 18, wherein the peptide is non-covalently bonded with iloprost or a pharmaceutically acceptable salt or stereoisomer thereof.

20. The pharmaceutical composition of claim 18 or 19, wherein the peptide comprises a primarily acidic peptide fragment and a primarily basic peptide fragment, with a neutral peptide fragment in between the acidic and basic fragments.

21. The pharmaceutical composition of any one of claims 18-20, wherein the ratio of iloprost or a pharmaceutically acceptable salt or stereoisomer thereof to the peptide is in the range of about 1:2 to about 1:10.

22. The pharmaceutical composition of any one of claims 18-21, wherein the composition comprises a solvent selected from water, tetrahydrofuran, dimethylformamide, dimethylsulfoxide or acetonitrile, or a combination thereof.

23. The pharmaceutical composition of any one of claims 1-5, wherein the composition comprises a liposome.

24. The pharmaceutical composition of claim 23, wherein the liposome comprises phosphatidyl choline, phosphatidyl glycerol, phosphatidyl serine, phosphatidic acid, cholesterol, cholesterol derivatives, plant sterol, phosphatidyl ethanolamine, distearoylphophatidyl ethanolamine, pegylated phosphatidyl ethanolamine, pegylated distearoylphosphatidyl ethanolamine, neutral phospholipid, neutral lipid, cationic lipids, amphipathic lipid, charged phospholipid, or combinations thereof.

25. The pharmaceutical composition of claim 23, wherein the liposome comprises a lipid, a phospholipid, a cholesterol, or a combination thereof.

26. The pharmaceutical composition of any one of claim 23-25, wherein the liposome comprises PEG-distearoylphosphatidyl ethanolamine, dimyristoylphosphatidyl choline (DMPC), distearoylphosphatidyl choline (DSPC), dipalmitoyl phosphatidyl choline (DPPC), dimyristoylphosphatidyl glycerol (DMPG), dipalmitoylphosphatidyl glycerol (DPPG), distearoylphosphatidyl glycerol (DSPG), dimyristoylphosphatidyl serine (DMPS), dipalmitoylphosphatidyl serine (DPPS), distearoylphosphatidyl serine (DSPS), dimyristoylphosphatidic acid (DMPA), dipalmitoylphosphatidic acid (DPPA), cholesterol hemisuccinate (CHEMS), hydrogenated soy bean phosphatidyl choline (HSPC), distearoyl phosphatidyl choline (DSPC), hydrogenated egg phosphatidyl choline (HEPC), soy bean phosphatidyl choline (SPC), egg phosphatidyl choline (EPC), dilauroyl phosphatidylglycerol, dipalmitoyl phosphatidylglycerol, distearoyl phosphatidyl glycerol, dimyristate phosphatidylglycerol, dioleic acid phosphatidylserine, dioleoyl phosphatidylglycerol, dilauroyl phosphatidic acid, dimyristate phosphatidic acid, distearoyl phosphatidic acid, or combinations thereof.

27. The pharmaceutical composition of any one of claims 23-26, wherein the iloprost or a pharmaceutically acceptable salt or stereoisomer thereof is entrapped or encapsulated in the liposome.

28. The pharmaceutical composition of any one of claims 23-27, wherein the liposome has a mean particle diameter in the range of about 10 nm to about 1,000 nm.

29. The pharmaceutical composition of any one of claims 23-27, wherein the liposome has a mean particle diameter in the range of about 10 nm to about 300 nm.

30. The pharmaceutical composition of any one of claims 1-5, wherein the composition comprises microspheres.

31. The pharmaceutical composition of claim 30, wherein the microspheres are polymer microspheres.

32. The pharmaceutical composition of claim 30 or 31, wherein the microspheres comprise polylactide polymer, sodium acrylate polymer, acrylamide polymer, acrylamide derivative polymer or copolymer, sodium acrylate and vinyl alcohol copolymer, a vinyl acetate and acrylic acid ester copolymer, a vinyl acetate and methyl maleate copolymer, and a isobutylene-maleic anhydride crosslinked copolymer, polylactic acid (PLA), polyglycolic acid (PGA), a copolymer of PLA and PGA, poly-L-lactide (PLLA), poly-D,L-lactide (PDLA), poly-capralactone (PCL), poly(D,L-lactide-co-glycolide), poly(3-hydroxy-butyrate), polylactone, polyanhydride, poly(hydroxy-butyrate)-co-(hydroxy-valerate), polypropylene-glucose, poly(lactic acid)-polyglycol, and poly(hydroxyacetic acid)-polyglycol, or a combination thereof.

33. The pharmaceutical composition of any one of claims 30-32, wherein the microspheres comprise a biodegradable wall.

34. The pharmaceutical composition of any one of claims 30-33, wherein the microspheres have a mean diameter of about 10 μm to about 300 μm.

35. The pharmaceutical composition of any one of claims 30-34, wherein the iloprost or a pharmaceutically acceptable salt or stereoisomer thereof is entrapped or encapsulated in the microsphere.

36. The pharmaceutical composition of any one of claims 31-35, wherein the composition comprises a gel.

37. The pharmaceutical composition of claim 36, wherein the gel is polymer gel.

38. The pharmaceutical composition of claim 36 or 37, wherein the gel comprises xanthan gum, deacylated xanthan gum, a carboxymethyl ether, propylene glycol ester, homo polysaccharide gum, galactomannan gum, hydroxypropyl cellulose, hydroxypropylmethyl cellulose, polylactide polymer, sodium acrylate polymer, acrylamide polymer, acrylamide derivative polymer or copolymer, sodium acrylate and vinyl alcohol copolymer, a vinyl acetate and acrylic acid ester copolymer, a vinyl acetate and methyl maleate copolymer, and a isobutylene-maleic anhydride crosslinked copolymer, polylactic acid (PLA), polyglycolic acid (PGA), a copolymer of PLA and PGA, poly-L-lactide (PLLA), poly-D,L-lactide (PDLA), poly-capralactone (PCL), poly(D,L-lactide-co-glycolide), poly(3-hydroxy-butyrate), polylactone, polyanhydride, poly(hydroxy-butyrate)-co-(hydroxy-valerate), polypropylene-glucose, poly(lactic acid)-polyglycol, and poly(hydroxyacetic acid)-polyglycol, or a combination thereof.

39. The pharmaceutical composition of any one of claims 36-37, wherein the iloprost or a pharmaceutically acceptable salt or stereoisomer thereof is entrapped or encapsulated in the gel.

40. The pharmaceutical composition of any one of claims 1-5, wherein the composition is a phospholipid gel.

41. A pharmaceutical composition for injection, comprising iloprost or a pharmaceutically acceptable salt or stereoisomer thereof and a pharmaceutically acceptable carrier or excipient;
wherein a single daily injection provides maximum serum concentration (Cmax) of iloprost is in the range of about 10 pg/mL to about 150 pg/mL and at least about 8 hours of iloprost-free period within 48 hours from each injection.

42. The pharmaceutical composition of claim 41, wherein the Cmax of iloprost is in the range of about 20 pg/mL to about 40 pg/mL.

43. The pharmaceutical composition of claim 41, wherein the Cmax of iloprost is in the range of about 40 pg/mL to about 60 pg/mL.

44. The pharmaceutical composition of claim 41, wherein the Cmax of iloprost is in the range of about 60 pg/mL to about 80 pg/mL.

45. The pharmaceutical composition of claim 41, wherein the Cmax of iloprost is in the range of about 80 pg/mL to about 130 pg/mL.

46. The pharmaceutical composition of claim 41, wherein the composition provides a serum concentration of iloprost at steady state (Css) is in the range of about 10 pg/mL to about 30 pg/mL.

47. The pharmaceutical composition of claim 41, wherein the composition provides a Css of iloprost is in the range of about 30 pg/mL to about 60 pg/mL.

48. The pharmaceutical composition of claim 41, wherein the composition provides a Css of iloprost is in the range of about 60 pg/mL to about 80 pg/mL.

49. The pharmaceutical composition of claim 41, wherein the composition provides a Css of iloprost is in the range of about 80 pg/mL to about 120 pg/mL.

50. The pharmaceutical composition of any one of claims 41-49, wherein the composition provides a daily serum concentration of iloprost at steady state (Css) for about 6 hours to about 24 hours.

51. The pharmaceutical composition of any one of claims 41-49, wherein a daily serum concentration of iloprost at steady state (Css) for about 6 hours.

52. The pharmaceutical composition of any one of claims 41-51, wherein the iloprost-free period is a duration in which iloprost is undetectable in the subject's plasma.

53. The pharmaceutical composition of any one of claims 41-52, wherein the single daily injection does not result in daily accumulation of iloprost in the subject.

54. The pharmaceutical composition of claim 41, wherein the composition provides an area under the plasma concentration versus time curve from time of dosing to the last time point with measurable concentration serum concentration (AUC₀₋ₗₐₛₜ) of iloprost in the range of about 100 pg·hr/mL to about 200 pg·hr/mL.

55. The pharmaceutical composition of claim 41, wherein an AUC₀₋ₗₐₛₜ of iloprost is in the range of about 200 pg·hr/mL to about 350 pg·hr/mL.

56. The pharmaceutical composition of claim 41, wherein an AUC₀₋ₗₐₛₜ of iloprost is in the range of about 350 pg hr/mL to about 500 pg·hr/mL.

57. The pharmaceutical composition of claim 41, wherein an AUC₀₋ₗₐₛₜ of iloprost is in the range of about 500 pg·hr/mL to about 700 pg·hr/mL.

58. The pharmaceutical composition of claim any one of claims 41-57, wherein the injection is a subcutaneous injection.

59. The pharmaceutical composition of any one of claims 41-58, wherein the single daily dose has a volume of less than about 1.0 mL.

60. The pharmaceutical composition of claim 41, wherein the single daily dose comprises about 100 ng iloprost or a pharmaceutically acceptable salt or stereoisomer thereof per weight of the subject (kg) to about 200 ng/kg.

61. The pharmaceutical composition of claim 41, wherein the single daily dose comprises iloprost or a pharmaceutically acceptable salt or stereoisomer thereof in about 200 ng/kg to about 400 ng/kg.

62. The pharmaceutical composition of claim 41, wherein the single daily dose comprises iloprost or a pharmaceutically acceptable salt or stereoisomer thereof in about 400 ng/kg to about 600 ng/kg.

63. The pharmaceutical composition of claim 41, wherein the single daily dose comprises iloprost or a pharmaceutically acceptable salt or stereoisomer thereof in about 600 ng/kg to about 800 ng/kg.

64. The pharmaceutical composition of any one of claims 41-63, wherein the single daily dose comprises iloprost or a pharmaceutically acceptable salt or stereoisomer thereof in about 0.01% to about 20% by weight of the composition.

65. The pharmaceutical composition of any one of claims 41-64, wherein the composition comprises a phospholipid.

66. The pharmaceutical composition of claim 65, wherein the phospholipid is lecithin, phosphatidylcholine, phosphotidylethanolamine, phosphotidylserine, phosphatidylinositol, phosphoglyceride, phosphoglycerol, phospholipid, sphingosine, ganglioside, phytosphingosine, diacylglycerol, phosphocholine, phosphoethanolamine, hosphotidylserine, lysophospholipid, pegylated phospholipid, mixed chain phospholipid, or a combinations thereof.

67. The pharmaceutical composition of claim 66, wherein the lecithin is soy lecithin, egg lecithin, or a combination thereof.

68. The pharmaceutical composition of any one of claims 65-67, wherein the composition comprises a phospholipid in about 20% to about 80% by weight of the composition.

69. The pharmaceutical composition of any one of claims 41-68, wherein the composition comprises an oil.

70. The pharmaceutical composition of claim 69, wherein the oil is synthetic oil, a vegetable oil, sesame oil, a medium chain oil, silicone oil, ethyl oleate, fatty acid, vitamin E, vitamin E succinate, cholesterol, triglyceride oil, or a mixture thereof.

71. The pharmaceutical composition of any one of claims 41-70, wherein the pharmaceutically acceptable excipient is sucrose, dextrose, lactose, glucose, trehalose, maltose, mannitol, sorbitol, glycerol, amylose, starch, amylopectin, triglycerides, fatty acids, carbohydrates, or a mixture thereof.

72. The pharmaceutical composition of any one of claims 41-71, wherein the pharmaceutically acceptable excipient is an acidifying agent, an alkalizing agent, a pH buffering agent, a metal ion chelator, an antioxidant, a preservative, a tonicity/osmotic pressure modifier, a condensing agent, a solubilizing agent, or a mixture thereof.

73. The pharmaceutical composition of any one of claims 41-72, wherein the pharmaceutically acceptable carrier is ethanol, propylene glycol, glycerol, sorbitol, polyethylene glycol, silicone oil, glycofurol, ethyl oleate, or a mixture thereof.

74. The pharmaceutical composition of any one of claims 41-73, wherein the composition is an injectable gel composition.

75. The pharmaceutical composition of any one of claims 41-74, wherein the composition is a nanoemulsion.

76. The pharmaceutical composition of any one of claims 41-75, wherein the composition is bioequivalent to iloprost continuous infusion over 6 hours each day for 5 consecutive days at 0.5 to 2.0 ng iloprost/kg body weight/min.

77. A pharmaceutical composition of any one of claims 41-76 for use in combination with one or more days of treatment with iloprost continuous infusion over 6 hours/day at 0.5 to 2.0 ng iloprost/kg body weight/min.

78. The pharmaceutical composition for use of claim 77, wherein the use is in combination with one day of treatment with iloprost continuous infusion over 6 hours/day at 0.5 to 2.0 ng iloprost/kg body weight/min.

79. The pharmaceutical composition for use of claim 77 or 78, wherein the composition is administered after the treatment with iloprost continuous infusion over 6 hours/day at 0.5 to 2.0 ng iloprost/kg body weight/min, wherein the administration of the composition is on the same day or on a different day than the treatment with iloprost continuous infusion.

80. The pharmaceutical composition for use of claim 77 or 78, wherein the composition is administered starting the day after the treatment with iloprost continuous infusion over 6 hours/day at 0.5 to 2.0 ng iloprost/kg body weight/min.

81. The pharmaceutical composition of any one of claims 1-80, wherein the composition has a pH in the range of about 8.0 to about 8.9.

82. A method of treating symptomatic Raynaud's Phenomenon (RP) in a subject with systemic sclerosis (SSc), comprising administering the pharmaceutical composition of any one of claims 1-81.

83. The method of claim 82, wherein the method reduces the frequency of symptomatic RP episodes.

84. The method of claim 82, wherein the method reduces the average duration of symptomatic RP episodes.

85. The method of claim 82, wherein the method reduces the severity of symptomatic RP episodes.

86. The method of claim 82, wherein the method increases the number of days without symptomatic RP attacks.

87. The method of claim 82, wherein the method reduces the pain in the subject's fingers associated with symptomatic RP episodes.

88. The method of claim 82, wherein the method reduces the severity of numbness in the subject's fingers associated with symptomatic RP episodes.

89. The method of claim 82, wherein the method reduces the severity of tingling in the subject's fingers associated with symptomatic RP episodes.

90. The method of claim 82, wherein the method reduces the severity of discomfort in the subject's fingers associated with symptomatic RP episodes.

91. A method of preventing the development of digital ischemia in a subject with systemic sclerosis (SSc), comprising administering the pharmaceutical composition of any one of claims 1-81.

92. The method of claim 91, wherein the digital ischemia is critical digital ischemia.

93. A method of preventing the development of RP episodes in a subject with systemic sclerosis (SSc), comprising administering the pharmaceutical composition of any one of claims 1-81.

94. A method of treating and preventing the development of digital infection, digital ischemic lesion, digital ischemic ulcer, or gangrene in a subject with systemic sclerosis (SSc), comprising administering the pharmaceutical composition of any one of claims 1-81.

95. The method of any one of claims 82-94, wherein the pharmaceutical composition of any one of claims 1-36 is administered in combination with one or more days of treatment with iloprost continuous infusion over 6 hours/day at 0.5 to 2.0 ng iloprost/kg body weight/min.

96. The method of claim 95, wherein the pharmaceutical composition is administered in combination with one day of treatment with iloprost continuous infusion over 6 hours/day at 0.5 to 2.0 ng iloprost/kg body weight/min.

97. The method of claim 95 or 96, wherein the pharmaceutical composition is administered after the treatment with iloprost continuous infusion over 6 hours/day at 0.5 to 2.0 ng iloprost/kg body weight/min, wherein the administration of the pharmaceutical composition is on the same day or on a different day than the treatment with iloprost continuous infusion.

98. The method of claim 95 or 96, wherein the pharmaceutical composition is administered starting the day after the treatment with iloprost continuous infusion over 6 hours/day at 0.5 to 2.0 ng iloprost/kg body weight/min.
